(19) United States
(12) Patent Application Publication
(10) Pub. No.: US 2013/0171677 A1
(43) Pub. Date: Jul. 4, 2013

(54) RECOMBINANT PHYCOBILIPROTEINS WITH ENHANCED FLUORESCENCE AND PHOTOCHEMICAL PROPERTIES

(71) Applicants: Donald A. Bryant, Port Matilda, PA (US); Wendy M. Schluchter, New Orleans, LA (US); Richard M. Alvey, Honolulu, HI (US); Avijit Biswas, Hattiesburg, MS (US)

(72) Inventors: Donald A. Bryant, Port Matilda, PA (US); Wendy M. Schluchter, New Orleans, LA (US); Richard M. Alvey, Honolulu, HI (US); Avijit Biswas, Hattiesburg, MS (US)

(73) Assignees: BOARD OF SUPERVISORS OF THE UNIVERSITY OF LOUISIANA SYSTEM, New Orleans, LA (US); THE PENN STATE RESEARCH FOUNDATION, University Park, PA (US)

(21) Appl. No.: 13/821,494

(22) PCT Filed: Sep. 8, 2011
(86) PCT No.: PCT/US11/50753

§ 371 Date: Mar. 7, 2013

Related U.S. Application Data
(60) Provisional application No. 61/402,955, filed on Sep. 8, 2010.

(51) Int. Cl. C07K 14/00

(52) U.S. Cl. CPC C07K 14/00
USPC 435/23; 530/350; 435/69.1; 435/325; 435/254.2; 435/252.3; 435/252.33; 435/232; 436/501

(57) ABSTRACT

Novel fluorescent phycobiliprotein fusion proteins and methods of use are described. The novel phycobiliproteins can be produced in a cell comprising two or more heterodimeric lyases, an apoprotein and a bilin reductase, which components react to form the phycobiliprotein fusion protein. Also described are phycobiliprotein based transcription reporter cells and assays, which cells conditionally express a heterologous, fluorescent, phycobiliprotein domain even in anoxic conditions.

### CROSS-REFERENCE TO RELATED APPLICATIONS

This application claims priority under 35 U.S.C. §119 to provisional application Ser. No. 61/402,955 filed Sep. 8, 2010, herein incorporated by reference in its entirety.

### GRANT REFERENCE

This invention was made with government support under Contract No. 0519743, 0133441, and 0843664 awarded by National Science Foundation. The government has certain rights in the invention.

### BACKGROUND OF THE INVENTION

The use of fluorescent proteins has revolutionized many areas of biology. A primary example is the Green Fluorescent Protein or GFP and its many derivatives. Protein tags have been particularly useful due to their relatively small size, bright fluorescence, the variety of colors and the fact that the tag can be introduced as a single gene or gene fusion. The primary limitation of the use of GFP is the requirement for oxygen in the synthesis of the chromophore. This limits the usefulness for those wishing to utilize fluorescent technologies in anaerobes or cells that have been grown in anoxic conditions.

The phycobiliproteins are a family of light-harvesting proteins found in cyanobacteria, red algae, and the cryptomonads. These proteins absorb strongly in the visible region of the spectrum because they carry various covalently attached linear tetrapyrrole prosthetic groups (bilins). Their ability to absorb and conduct light energy to chlorophylls is due to the posttranslational covalent attachment of linear tetrapyrrole chromophore(s) to cysteine residue(s) in the chromophore binding pockets(s) of these proteins. The highly efficient transfer of energy to chlorophylls of the photosystems in the natural system causes them to have nearly no fluorescence when properly assembled in vivo, but when phycobilisomes are purified from cells, they are highly fluorescent due to the absorption of light energy and the inability to transfer that energy to downstream chlorophyll acceptors.

The invention offers numerous applications in the enzymology and chemistry of phycobiliprotein synthesis, and enables the use of phycobiliproteins as in vivo fluorescent protein markers in the absence of oxygen. Purified native phycobiliproteins and their subunits fluoresce strongly and have been widely used as external labels for cell sorting and analysis and a wide range of other fluorescence-based assays. Fluorescent protein markers expressed in vivo, such as Aequorea victoria green fluorescent protein and its variants, have proved to be of great value in all fields of cell biology. Phycobiliprotein constructs represent a broad array of spectroscopically distinctive proteins with photophysical properties superior to those of fluorescent markers currently available. The expression in prokaryotic or eukaryotic cells of genes encoding enzymes and apo-phycobiliprotein subunit-containing fusion proteins permits intracellular production of constructs carrying specific bilins at unique locations, with broad application.

### SUMMARY OF THE INVENTION

The invention provides fluorescent phycobiliproteins which have been modified to have improved properties for use in assays. According to the invention, apoproteins such as CpcA and PecA may be combined with different lyases to attach chromophores to the same. This use of novel combinations of chromophores, apoproteins and lyases leads to the synthesis of novel phycobiliproteins with different properties that may be taken advantage of for use in various reporter assays. This is surprising as traditionally it was thought that the lyase-protein and lyse-bilin interaction was highly specific; see one or more of the following: Biswas, A., Y. M. Vasquez, T. M. Dragomani, M. L. Kronfel, S. R. Williams, R. M. Alvey, D. A. Bryant, and W. M. Schluchter. 2010, Biosynthesis of Cyanobacterial Phycobiliproteins in Escherichia coli: Chromophorylation Efficiency and Specificity of All Bilin Lyases from Synechococcus sp Strain PCC 7002, Applied and Environmental Microbiology 76:2729-2739; Fairchild, C. D., J. Zhao, J. Zhou, S. E. Colson, D. A. Bryant, and A. N. Glazer, 1992, Phycocyanin α subunit phycocyanobilin lyase, Proc. Natl. Acad. Sci., USA 89:7017-7021; Fairchild, C. D., and A. N. Glazer. 1994, Oligomeric structure, enzyme kinetics, and substrate specificity of the phycocyanin alpha subunit phycocyanobilin lyase, J Biol Chem 269:8686-8694; Saunée, N. A., S. R. Williams, D. A. Bryant, and W. M. Schluchter, 2008, Biogenesis of phycobiliproteins, II, CpcS-I and CpcU comprise the heterodimeric bilin lyase that attaches phycocyanobilin to Cys-82 of beta-phycocyanin and Cys-81 of allophycocyanin subunits in Synechococcus sp. PCC 7002, J. Biol. Chem. 283:7513-7522; Scheer, H., and K. H. Zhao. 2008, Biliprotein maturation: the chromophore attachment, Molecular Microbiology 68:263-276; Schluchter, W. M., Shen, G., Alvey, R. M., Biswas, A., Saunée, N. Williams, S. R., Miller, C. A., and D. A. Bryant (2010), Phycobiliprotein Biosynthesis in Cyanobacteria: Structure and Function of Enzymes Involved in Post-translational Modification In Recent Advances in Phototrophic Prokaryotes, Series: Advances in Experimental Medicine & Biology; Patrick C. Hallenbeck (ed) Springer. NY, N.Y.; Shen, G., W. M. Schluchter, and D. A. Bryant. 2008. Biogenesis of phycobiliproteins, I, cpcS-I and cpcU mutants of the cyanobacterium Synechococcus sp. PCC 7002 define a heterodimeric phycocaynobilin lyase specific for beta-phycocyanin and allophycocyanin subunits, J. Biol. Chem. 28:7503-7512; Zhao, K. H., P. Su, J. Tu, X. Wang, H. Liu, M. Ploscher, L. Eichacker, B. Yang, M. Zhou, and H. Scheer. 2007. Phycobilin:cystein-84 biliprotein lyase, a near-universal lyase for cysteine-84-binding sites in cyanobacterial phycobiliproteins. Proc Natl Acad Sci USA 104:14300-14305.

The novel combinations of lyases, apoproteins, and chromophores, in the presence of a bilin reductase also leads to assays and production of biliproteins in anoxic conditions, a limitation of heretofore known reporter proteins such as Green Fluorescent Protein.

According to the invention the lyase CpcE/CpcF has been shown to attach the chromophores PEB and PΦB to create the novel phycobiliproteins HT-CpcA-PEB, and HT-CpcA-PΦB.

In a further embodiment, the lyase PecE/PecF was used to attach other chromophores to CpcA to create the novel phycobiliproteins HT-CpcA-PVB, and HT-CpcA-PUB. In a further embodiment, when HY2 was the bilin reductase, the novel phycobiliprotein HT-CpcA-PtVB was produced. PtVB is not only a novel chromophore to attach to CpcA, but it is also not known to occur as a natural chromophore for any type of biliprotein.

The invention contemplates the use of still other lyases such as CpcS from Thermosynechococcus elongatus (also known as Ter13), CpcS/CpcU from Synechocococcus sp. PCC 7002, and CpeS which attaches PEB to CpeB. See Biswas et al., 2011. JBC and the like along with other apoprotein and bilin combinations to produce unique and novel phycobiliproteins with properties that can be used with advantage in reporter assays.

The invention also provides recombinant cells which produce a fluorescent phycobiliproteins and methods of use. Cells which do not naturally express the same are genetically engineered to comprise a functional pathway for making a fluorescent phycobiliprotein that is not oxygen dependent. In a particular embodiment, the cell makes and comprises a lyase, the apoprotein CpcA and a bilin reductase, such as PcyA, PebS, or HY2. In another embodiment, the bilin reductase can be supplanted by the exogenous addition of the end product linear tetrapyrrole.

The invention may be practiced in a wide variety of cells, including mammalian cells, yeast cells (e.g. S. cerevisiae), bacterial cells (e.g. E. coli), etc., which may be present in vitro, which are generally isolated from a host, or in situ.

The invention also provides methods of making and using the subject cells and fusion proteins. For example, subject methods include making phycobiliprotein fusion proteins by growing the subject recombinant cells under conditions wherein the cells express the same, which methods may further comprise the step of isolating the phycobiliprotein fusion protein, and/or the step of specifically detecting, including detecting the location, movement, interactions, appearance, or catabolism of the phycobiliprotein fusion protein, particularly within the cell.

In another embodiment, the invention provides phycobiliprotein based transcription reporter cells and assays. For example, the invention includes a recombinant cell which conditionally expresses a heterologous, fluorescent, phycobiliprotein and comprises CpcA (encoding the bilin acceptor protein), and a lyase, either CpcE/CpcF or PecE/PecF. In a particularly preferred embodiment, the reporter cell and assay is in an anoxic environment and the assay includes the introduction of a source of biliverdin.

### DESCRIPTION OF THE FIGURES

FIG. 1 shows the absorption spectra and fluorescence emission spectra of CpcA with the three different 3-ethylidine chromphores along with a photograph of the purified protein. A. PCB-CpcA, B. PEB-CpcA, and C. PΦB-CpcA.

FIG. 2 shows the attachment of isomerized chromophores to CpcA when the lyases are switched from CpcEF to PecEF. Absorption spectra and fluorescence emission spectra of CpcA with the three different 3-vinyl chromophores along with a photograph of the purified protein. A. PVB-CpcA, B. PUB-CpcA, and C. PtVB-CpcA.

FIG. 3 is a zinc stained and Coomassie blue stained polyacrylamide gel of each form of CpcA. Images of Zinc stained (top) and Coomassie blue (bottom) stained polyacrylamide gel containing each form of CpcA Lane 1: PCB-CpcA, Lane 2: PVB-CpcA, Lane 3: PEB-CpcA, Lane 4: PUB/PEB-CpcA, Lane 5: PΦB-CpcA, Lane 6: PtVB-CpcA.

FIG. 4 is a demonstration of photochemistry of PVB-CpcA and PtVB-CpcA Absorbance profile of PVB-CpcA (A) and PtVB-CpcA (B) before (solid line) and after (dashed line) treatment with saturating light at its pre-treatment absorption maxima and the difference spectra (C.) of the two forms of PVB-CpcA (solid line) and PtVB (dashed line).

FIG. 5 shows CpcA with PcyA and chimeric lyases. Absorbance profile of proteins isolated from strains expressing HO1 pcyA and either CpcA pecEcpcF (dashed line) or CpcA CpcEpecF (solid line) (A) and pellets from E. coli cells expressing either CpcA/CpcEpecF or CpcA/pecE/CpcF and HO1/pcyA.

FIG. 6 is a depiction of the pathway for the synthesis of linear tetrapyrrole chromophores in cyanobacteria, algae, and plants.

FIG. 7 demonstrates that the pcyA gene is apparently essential in Synechococcus sp. strain PCC 7002. A. Colonies of wild-type Synechococcus sp. strain PCC 7002. B. Colonies of Synechococcus sp. strain PCC 7002 transformed with pcyA::aadA on medium containing streptomycin. Sectors and colonies with wild-type coloration are obvious. C. PCR amplification of the pcyA locus from wild type (lane 1) and the pcyA::aadA transformed strain (lane 2) of Synechococcus sp. strain PCC 7002. The two amplicons visible in lane 2 indicate that both pcyA and pcyA::aadA alleles are present in the genomic DNA of the transformed cells.

FIG. 8 shows overexpression of pebA and pebB in Synechococcus sp. strain PCC 7002. A. Colonies of Synechococcus sp. strain PCC 7002 transformed with the pebAB overexpression cassette mostly exhibit a reddish-brown phenotype resembling strains that naturally synthesize PE. A colony that has reverted to the wild-type color phenotype can be seen (arrow). B. Whole-cell absorption spectra of wild-type Synechococcus sp. strain PCC 7002 (solid line) and the strain overexpressing pebA and pebB.

FIG. 9 shows overexpression of pebA and pebB causes PEB to be ligated preferentially to the α subunit of PC. A. Coomassie Brilliant blue and zinc-enhanced fluorescence images of a single SDS-PAGE. Lane 1, recombinant CpcA carrying PCB; Lane 2, recombinant CpcA carrying PEB; Lane 3, PBS isolated from wild-type cells of Synechococcus sp. strain PCC 7002; Lane 4, PBSs isolated from cells of Synechococcus sp. strain PCC 7002 overexpressing pebA and pebB. B. Absorption spectra of major PBP subunits separated by HPLC from PBS isolated from wild-type Synechococcus sp. strain PCC 7002. C. Absorption spectra of major PBP subunits separated by HPLC from PBS isolated from cells of Synechococcus sp. strain PCC 7002 overexpressing pebA and pebB. Note that the CpcA subunit mostly carries a PEB chromophore with absorption at ˜560 nm, although some absorption from PCB is also evident.

FIG. 10 is the absorption spectra of Synechococcus sp. strain PCC 7002 expressing HY2. Absorption spectra of Synechococcus sp. strain PCC 7002 wild-type cells (solid line) and of a strain in which HY2 expression is being driven by the cpcBA promoter of Synechocystis sp. strain PCC 6803.

FIG. 11 shows that HY2 functionally substitutes for pcyA in Synechococcus sp. strain PCC 7002. A. Whole-cell absorption spectra of wild-type Synechococcus sp. strain PCC 7002 (solid line) and a strain overexpressing HY2, in which pcyA has been inactivated (dashed line). B. PCR analysis of the pcyA locus in wild-type Synechococcus sp. strain PCC 7002 (lane 1), a strain overexpressing HY2 in which pcyA has been deleted (lane 2), and the pcyA partial deletion strain (lane 3).

FIG. 12 is the 77 K fluorescence emission spectra of Synechococcus sp. strain PCC 7002 wild-type cells (solid line) and cells of the pcyA deletion strain overexpressing HY2 (dashed line). The excitation wavelength was 590 nm.

FIG. 13 depicts analysis of isolated PBS from wild type and a ΔpcyA::addA strain overproducing HY2. A. PAGE analysis of isolated PBS stained with either Coomassie brilliant blue or zinc chloride solutions. Absorption spectra (B) and 77 K fluorescence emission spectra (C) of PBSs isolated from wild type (solid line) and a ΔpcyA::addA strain overproducing HY2 (dashed line). The excitation wavelength was 590 nm.

FIG. 14 show the growth rate analysis of wild-type Synechococcus sp. strain PCC 7002 and its ΔpcyA::aadA strain overproducing HY2 (ΔpcyA::HY2). Cells were grown under otherwise standard conditions at high (supra-saturating) (500 μmol photons m [symbol] -2 s-1), medium (nearly saturating) (200 μmol photons m [symbol] -2 s-1) and low (limiting) (50 μmol photons m [symbol] -2 s-1) light intensities. The plotted data are the average of the results for at least three replicate cultures, and the calculated doubling times are indicated.

FIG. 15 demonstrates that the addition of exogenous commercially produced biliverdin to anoxically grown E. coli enables the production of functional holo-HT-PEB-CpcA. Phase contrast and corresponding fluorescent micrographs of E. coli cells induced to express HO1, pebS, CpcA, CpcE and CpcF under standard aerobic conditions (A), anoxic conditions (B) or anoxic conditions in which exogenous biliverdin has been added at the time of induction.

FIG. 16 is a flow cytometry analysis of the cultures from FIG. 15. Red line, culture from A. Black line, culture from B. Green line, culture from C.

FIG. 17 shows analyses of CpeA produced with CpeY and CpeZ in E. coli. A. Absorbance (solid line) and fluorescence emission (dashed line) spectra of CpeA purified from cells containing pCpeA, pPebS with pCpeYZ and absorbance (dashed dotted line), fluorescence (dotted line) without pCpeYZ are shown. B. Absorbance (solid line) and fluorescence emission (dashed line) spectra of CpeA purified from cells containing pCpeA, pPebS with pCpeY and absorbance (dashed dotted line), fluorescence (dotted line) with pCpeZ are shown. In order to acquire the fluorescence emission spectra for the CpeA produced in the presence of pCpeYZ and pCpeY (dashed lines in panels A and B) the samples were diluted fifteen- and eight-fold, respectively, to OD560 nm of 0.05; however, no dilution was performed on CpeA produced in the absence of a lyase or in the presence of pCpeZ (dotted lines in panels A and B). C. SDS-PAGE analysis of recombinant CpeA. Lane 1, CpeA purified from cells containing pCpeA, pPebS with no lyase; lane 2, CpeA purified from cells containing pCpeA, pPebS and pCpeYZ; lane 3, CpeA purified from cells containing pCpeA, pPebS, and pCpeY; lane 4, CpeA purified from cells containing pCpeA, pPebS, and pCpeZ. Molecular mass standards are loaded in lane “S”, and the mass is indicated to the right. D. The zinc-enhanced fluorescence of the gel pictured in panel C.

FIG. 18 shows analyses of the specific cysteine residue on CpeA required for PEB addition by CpeY/CpeZ. A. Absorbance (solid line) and fluorescence emission (dashed line) spectra of CpeA(C139S) purified from cells containing CpeA(C139S), pPebS with pCpeYZ and the absorbance (dashed dotted line), fluorescence (dotted line) spectra from cells containing CpeA(C82S), pPebS with pCpeYZ are shown. In order to acquire the fluorescence emission spectra for the HTCpeA (C139S) produced in the presence of pCpeYZ (dashed lines in panel A), the sample was diluted fifteen-fold to OD560 nm of 0.05; however, no dilution was performed on CpeA(C82S) (dotted line in panel A). B. and C. SDS-PAGE analysis of CpeA variants; gel was stained with Coomassie blue (B) or visualized by Zn-enhanced fluorescence (C). CpeA variants were produced in cells that also contained pPebS and pCpeYZ. Lane 1, CpeA (C82S); lane 2, CpeA (C139S); lane 3 CpeA (C82S,C139S). Molecular mass standards are loaded in lane “S”, and masses are indicated to the right. C. Zn-enhanced fluorescence image of the gel pictured in panel B.

FIG. 19 includes mass spectrometric analyses of tryptic peptides of CpeA-PEB produced with CpeY and CpeZ. A. MALDI MS/MS spectrum of the m/z 935 precursor ion derived from peptides resulting from the tryptic digestion of the covalent complex CpeA-PEB. This m/z 935 precursor ion was deduced to be a peptide fragment with a covalently bound PEB chromophore. B. Fragmentation pattern and corresponding mass assignments for data in panel A. A tick mark prior to number, e.g., '470, indicates one hydrogen has been transferred to the departing neutral upon cleavage. A tick mark after number, e.g., 814′, indicates the transfer of one hydrogen to the formed ion. A (.) indicates a radical ion.

FIG. 20 shows analyses of CpeA produced with CpeS in E. coli A. Absorbance (solid line) and fluorescence emission (dashed line) spectra of CpeA purified from cells containing pCpeA, pPebS with pCpeS B. Coomassie-stained SDS polyacrylamide gel containing CpeA purified from cells containing pCpeA, pPebS, and pCpeS. C. The zinc-enhanced fluorescence of the gel pictured in panel B.

FIG. 21 shows analyses of the cysteine residue on CpeA for PEB addition by CpeS. A. Absorbance (solid line) and fluorescence emission spectra (dotted line) of CpeA (C82S) variants purified from cells containing pCpeS and pPebS. B. Absorbance (solid line) and fluorescence emission spectra (dotted line) of CpeA (C139S) variant purified from cells containing pCpeS and pPebS. C. Coomassie-stained, SDSpolyacrylamide gel analysis of CpeA variants purified from cells containing pPebS, pCpeS and either pCpeA (C82S) (lane 1), pCpeA(C139S) (lane 2) or pCpeA (C82S,C139S) (lane 3) D. The Zn-enhanced fluorescence of the gel in panel C.

FIG. 22 shows analyses of the CpeB produced in the presence of different lyases in E. coli. A. Absorbance (solid line) and fluorescence emission (dashed line) spectra of CpeB purified from cells containing pCpeB, pPebS with pCpeS and absorbance (dashed dotted line), fluorescence (dotted line) without pCpeS (no lyase). B. Absorbance (solid line) and fluorescence emission (dashed line) spectra of CpeB purified from cells containing pCpeB, pPebS and pCpeYZ. In order to acquire the fluorescence emission spectra for the CpeB produced in the presence of pCpeS (dashed lines in panel A), the sample was diluted fifteen-fold to OD560 nm of 0.05. No dilution was performed on CpeB produced in the absence of a lyase or with pCpeYZ (dotted lines in panels A and B). C. Coomassie-stained SDS polyacrylamide gel containing CpeB purified from cells containing pCpeB, pPebS, and no lyase (lane 1) or from cells containing pCpeB, pPebS, and pCpeYZ, (lane 2), or from cells containing pCpeB, pPebS, and pCpeS (lane 3). Molecular mass standards are loaded in lane “S”, and mass is indicated to the right. D. Zn-enhanced fluorescence image of the gel pictured in panel C.

FIG. 23 shows analyses of the specific cysteine residue on CpeB required for PEB addition by CpeS: A. Absorbance (solid line) and fluorescence emission spectra (dotted line) of CpeB obtained by coexpressing pCpeB(C165S), pCpeS, and pPebS. The samples have been diluted fifteen-fold to an absorbance of 0.05. B. Absorbance (solid line) and fluorescence emission spectra (dotted line) of CpeB obtained by coexpressing pCpeB (C48S/C59S), pCpeS, and pPebS. C. Absorbance (solid line) and fluorescence emission spectra (dotted line) of CpeB obtained by coexpressing pCpeB (C80S), pCpeS, pPebS D. Coomassie-blue-stained SDS polyacrylamide gel loaded with CpeB purified from cells containing pCpeB (C48S/C59S), pPebS, and pCpeS (lane 1), pCpeB (C80S), pCpeS and pPebS (lane 2) or pCpeB (C165S), pPebS, pCpeS (lane 3). The electrophoretic mobility positions of molecular mass standards are indicated to the right. E. Zn-enhanced fluorescence image of the gel pictured in panel D.

FIG. 24 shows an alignment of TeCpcS, and CpcS and CpcU from Synechococcus sp. PCC 7002. The program T-coffee was used to align these protein sequences (Notredame, Higgins, Heringa 2000. T-Coffee: A novel method for multiple sequence alignments., JMB, 302; 205-217).

FIG. 25 shows that chromophorylation of CpcB by TeCpcS versus CpcS/CpcU. (A) Absorbance (solid) and fluorescence emission (dashed) spectra of HT-CpcBA purified from recombinant E. coli cells containing pCpcBA with pPcyA and expressing either TeCpcS (blue), CpcSU (red) or no lyase (black). (B) Coomassie-stained SDS-polyacrylamide gel of HT-CpcBA purified from cells containing pCpcBA, pPcyA and expressing either TeCpcS (lane 1 and lane 3) or CpcS/CpcU (lane 2). Low molecular mass standards were loaded in lane S. (C) Zinc-enhanced bilin fluorescence of the gel in panel B.

FIG. 26 depicts an analysis of purified HT-CpcB chromophorylated by TECpcS in recombinant E. coli. (A) Absorbance (solid red) and fluorescence emission (dashed red) spectra of HT-CpcB purified from cells containing pCpcBA, pCpcS and pPebS; absorbance (solid green) and fluorescence emission (dashed green) from cells containing pCpcBA, pCpcS and pHy2. (B) Coomassie-stained SDS-polyacrylamide gel of HT-CpcB purified from cells containing pCpcBA, pCpcS and either pPebS (lane 1), pHy2 (lane 2), or pPcyA (lane 3) and a low molecular weight standard ladder (lane S). (C) Zinc-enhanced bilin fluorescence of the gel in panel C.

FIG. 27 shows the attachment of three different bilins (PEB, PCB or PΦB) to AP by TECpcS. Absorbance (solid) and fluorescence emission (dashed) spectra of HT-ApcAB purified from recombinant E. coli cells containing pApcAB, pCpcS and either the bilin reductase genes to produce (A) PCB from pPcya, (B) PEB from pPebS or (C) PΦB from pHy2. (D) Coomassie-stained SDS-polyacrylamide gel of HT-ApcAB purified from cells containing pApcAB, pCpcS and either pPcyA (lane 1), pPebS (lane 2), or pHy2 (lane 3). A low weight molecular mass standard was loaded in lane S. (E) Zinc-enhanced bilin fluorescence of the gel in panel D.

FIG. 28 shows the attachment of three different bilins (PEB, PCB or PΦB) to AP-B by TECpcS. Absorbance (solid) and fluorescence emission (dashed) spectra of HT-ApcDB purified from recombinant E. coli cells containing pApcDB, pCpcS and either the bilin reductase genes to produce (A) PCB from pPcya, (B) PEB from pPebS or (C) PΦB from pHy2. (D) Coomassie-stained SDS-polyacrylamide gel of HT-ApcDB purified from cells containing pApcDB, pCpcS and either pPcyA (lane 1), pPebS (lane 2), or pHy2 (lane 3). A low weight molecular mass standard was loaded in lane S. (E) Zinc-enhanced bilin fluorescence of the gel in panel D.

FIG. 29 shows the attachment of PEB and/or PCB to ApcF (A) Absorbance (solid) and fluorescence emission (dashed) spectra of HT-ApcF purified from recombinant E. coli cells containing pApcF, pCpcS and either pPcyA (blue) or pPebS (red). (B) Absorbance spectrum (solid purple) of HT-ApcF purified from recombinant E. coli cells containing pApcF, pCpcS, and both pPcyA and pPebs. Fluorescence emission resulting from excitation of this sample at 490 nm (red-dotted line) is attributed to protein-bound PEB. Fluorescence emission resulting from excitation at 590 nm (blue-dotted line) is attributed to protein-bound PCB. (C) Coomassie-stained SDS-polyacrylamide gel of HT-ApcF purified from cells containing pApcF, pCpcS and either pPcyA (lane 1), pPebS (lane 2), or both pPcyA and pPebS (lane 3). A low weight molecular mass standard was loaded in lane S. (D) Zinc-enhanced bilin fluorescence of the gel in panel C.

### DETAILED DESCRIPTION OF THE INVENTION

Unless otherwise defined herein, scientific and technical terms used in connection with the invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well known and commonly used in the art. The methods and techniques are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol. I, CRC Press (1976); Handbook of Biochemistry: Section A Proteins, Vol. II, CRC Press (1976); Essentials of Glycobiology, Cold Spring Harbor Laboratory Press (1999).

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native inter-nucleoside bonds, or both. The nucleic acid can be in any topological conformation. For instance, the nucleic acid can be single-stranded, double-stranded, triple-stranded, quadruplexed, partially double-stranded, branched, hair-pinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced artificially, e.g., by human intervention. An “isolated nucleic acid” also includes a nucleic acid integrated into a host cell chromosome at a heterologous site and a nucleic acid construct present as an episome. Moreover, an “isolated nucleic acid” can be substantially free of other cellular material or substantially free of culture medium when produced by recombinant techniques or substantially free of chemical precursors or other chemicals when chemically synthesized.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. For example, a “recombinant 1-alkene synthase” can be a protein encoded by a heterologous 1-alkene synthase gene; or a protein encoded by a duplicate copy of an endogenous 1-alkene synthase gene; or a protein encoded by a modified endogenous 1-alkene synthase gene; or a protein encoded by an endogenous 1-alkene synthase gene expressed from a heterologous promoter; or a protein encoded by an endogenous 1-alkene synthase gene where expression is driven, at least in part, by an endogenous promoter different from the organism's native 1-alkene synthase promoter.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

A particular, non-limiting example of a mathematical algorithm utilized for the comparison of sequences is that of Karlin and Altschul (Proc. Natl. Acad. Sci. (1990) USA 87:2264-68; Proc. Natl. Acad. Sci. USA (1993) 90: 5873-77) as used in the NBLAST and XBLAST programs (version 2.0) of Altschul et al. (J. Mol. Biol. (1990) 215:403-10). BLAST nucleotide searches can be performed with the NBLAST program, score=100, wordlength=12 to obtain nucleotide sequences homologous to nucleic acid molecules of the invention. BLAST polypeptide searches can be performed with the XBLAST program, score=50, wordlength=3 to obtain amino acid sequences homologous to polypeptide molecules of the invention. To obtain gapped alignments for comparison purposes, Gapped BLAST can be utilized as described in Altschul et al. (Nucleic Acids Research (1997) 25(17):3389-3402). When utilizing BLAST and Gapped BLAST programs, the default parameters of the respective programs (e.g., XBLAST and NBLAST) can be used (http://www.ncbi.nlm.nih.gov). One skilled in the art may also use the ALIGN program incorporating the non-linear algorithm of Myers and Miller (Comput. Appl. Biosci. (1988) 4:11-17). For amino acid sequence comparison using the ALIGN program one skilled in the art may use a PAM120 weight residue table, a gap length penalty of 12, and a gap penalty of 4.

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 71%, 72%, 73%, 74%, 75%, 76%, 77%, 78%, 79%, 80%, 81%, 82%, 83%, 84%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6 times SSC (where 20 times SSC contains 3.0 M NaCl and 0.3 M sodium citrate), 1% SDS at 65° C. for 8-12 hours, followed by two washes in 0.2 times SSC, 0.1% SDS at 65° C. for 20 minutes. It will be appreciated by the skilled worker that hybridization at 65° C. will occur at different rates depending on a number of factors including the length and percent identity of the sequences which are hybridizing.

A preferred, non-limiting example of stringent hybridization conditions includes hybridization in 4 times sodium chloride/sodium citrate (SSC), at about 65-70° C. (or hybridization in 4 times SSC plus 50% formamide at about 42-50° C.) followed by one or more washes in 1 times SSC, at about 65-70° C. A preferred, non-limiting example of highly stringent hybridization conditions includes hybridization in 1 times SSC, at about 65-70° C. (or hybridization in 1 times SSC plus 50% formamide at about 42-50° C.) followed by one or more washes in 0.3 times SSC, at about 65-70° C. A preferred, non-limiting example of reduced stringency hybridization conditions includes hybridization in 4 times SSC, at about 50-60° C. (or alternatively hybridization in 6 times SSC plus 50% formamide at about 40-45° C.) followed by one or more washes in 2 times SSC, at about 50-60° C. Intermediate ranges e.g., at 65-70° C. or at 42-50° C. are also within the scope of the invention. SSPE (1 times SSPE is 0.15 M NaCl, 10 mM NaH₂PO₄, and 1.25 mM EDTA, pH 7.4) can be substituted for SSC (1 times SSC is 0.15 M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (T_{.m}) of the hybrid, where Tₘ is determined according to the following equations. For hybrids less than 18 base pairs in length, T_{.m} (° C.)=2(# of A+T bases)+4(# of G+C bases). For hybrids between 18 and 49 base pairs in length, T.ₘ (° C.)=81.5+16.6(log₁₀[Na⁺])+0.41 (% G+C)−(600/N), where N is the number of bases in the hybrid, and [Na⁺] is the concentration of sodium ions in the hybridization buffer ([Na⁺] for 1 times SSC=0.165 M).

The skilled practitioner recognizes that reagents can be added to hybridization and/or wash buffers. For example, to decrease non-specific hybridization of nucleic acid molecules to, for example, nitrocellulose or nylon membranes, blocking agents, including but not limited to, BSA or salmon or herring sperm carrier DNA and/or detergents, including but not limited to, SDS, chelating agents EDTA, Ficoll, PVP and the like can be used. When using nylon membranes, in particular, an additional, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C. (Church and Gilbert (1984) Proc. Natl. Acad. Sci. USA 81:1991-1995,) or, alternatively, 0.2 times SSC, 1% SDS.

The nucleic acids (also referred to as polynucleotides) may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “derived from” is intended to include the isolation (in whole or in part) of a polynucleotide segment from an indicated source. The term is intended to include, for example, direct cloning, PCR amplification, or artificial synthesis from, or based on, a sequence associated with the indicated polynucleotide source.

The term “gene” as used herein refers to a nucleotide sequence that can direct synthesis of an enzyme or other polypeptide molecule (e.g., can comprise coding sequences, for example, a contiguous open reading frame (ORF) which encodes a polypeptide) or can itself be functional in the organism. A gene in an organism can be clustered within an operon, as defined herein, wherein the operon is separated from other genes and/or operons by intergenic DNA. Individual genes contained within an operon can overlap without intergenic DNA between the individual genes.

An “isolated gene,” as described herein, includes a gene which is essentially free of sequences which naturally flank the gene in the chromosomal DNA of the organism from which the gene is derived (i.e., is free of adjacent coding sequences which encode a second or distinct polypeptide or RNA molecule, adjacent structural sequences or the like) and optionally includes 5′ and 3′ regulatory sequences, for example promoter sequences and/or terminator sequences. In one embodiment, an isolated gene includes predominantly coding sequences for a polypeptide.

The term “expression” when used in relation to the transcription and/or translation of a nucleotide sequence as used herein generally includes expression levels of the nucleotide sequence being enhanced, increased, resulting in basal or housekeeping levels in the host cell, constitutive, attenuated, decreased or repressed.

The term “codon usage” is intended to refer to analyzing a nucleic acid sequence to be expressed in a recipient host organism (or a cellular extract thereof) for the occurrence and use of preferred codons the host organism transcribes advantageously for optimal nucleic acid sequence transcription. The recipient host may be recombinantly altered with any preferred codon. Alternatively, a particular cell host can be selected that already has superior codon usage, or the nucleic acid sequence can be genetically engineered to change a limiting codon to a non-limiting codon (e.g., by introducing a silent mutation(s)).

The term “vector” as used herein is intended to refer to a nucleic acid molecule capable of transporting another nucleic acid to which it has been linked. One type of vector is a “plasmid,” which refers to a circular double stranded DNA loop into which additional DNA segments may be ligated. Other vectors include cosmids, bacterial artificial chromosomes (BAC) and yeast artificial chromosomes (YAC), fosmids, phage and phagemids. Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

“Expression optimization” as used herein is defined as one or more optional modifications to the nucleotide sequence in the promoter and terminator elements resulting in desired rates and levels of transcription and translation into a protein product encoded by said nucleotide sequence. Expression optimization as used herein also includes designing an effectual predicted secondary structure (for example, stem-loop structures and termination sequences) of the messenger ribonucleic acid (mRNA) sequence to promote desired levels of protein production. Other genes and gene combinations essential for the production of a protein may be used, for example genes for proteins in a biosynthetic pathway, required for post-translational modifications or required for a heteromultimeric protein, wherein combinations of genes are chosen for the effect of optimizing expression of the desired levels of protein product. Conversely, one or more genes optionally may be “knocked-out” or otherwise altered such that lower or eliminated expression of said gene or genes achieves the desired expression levels of protein. Additionally, expression optimization can be achieved through codon optimization. Codon optimization, as used herein, is defined as modifying a nucleotide sequence for effectual use of host cell bias in relative concentrations of transfer ribonucleic acids (tRNA) such that the desired rate and levels of gene nucleotide sequence translation into a final protein product are achieved, without altering the peptide sequence encoded by the nucleotide sequence.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

An isolated or purified polypeptide is substantially free of cellular material or other contaminating polypeptides from the expression host cell from which the polypeptide is derived, or substantially free from chemical precursors or other chemicals when chemically synthesized. In one embodiment, an isolated or purified polypeptide has less than about 30% (by dry weight) of contaminating polypeptide or chemicals, more advantageously less than about 20% of contaminating polypeptide or chemicals, still more advantageously less than about 10% of contaminating polypeptide or chemicals, and most advantageously less than about 5% contaminating polypeptide or chemicals.

The term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

Sequence homology may be measured by any common sequence analysis algorithm, such as Gap or Bestfit.

Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology—A Synthesis (Golub and Gren eds., Sinauer Associates, Sunderland, Mass., 2nd ed. 1991), which is incorporated herein by reference. Stereoisomers (e.g., D-amino acids) of the twenty conventional amino acids, unnatural amino acids such as .alpha.-, .alpha.-disubstituted amino acids, N-alkyl amino acids, and other unconventional amino acids may also be suitable components for polypeptides. Examples of unconventional amino acids include: 4-hydroxyproline, .gamma.-carboxyglutamate, .epsilon.-N,N,N-trimethyllysine, .epsilon.-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-331 and 25:365-389 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. (Pearson, Methods Enzymol. 183:63-98 (1990) (herein incorporated by reference). For example, percent sequence identity between amino acid sequences can be determined using FASTA with its default parameters (a word size of 2 and the PAM250 scoring matrix), as provided in GCG Version 6.1, herein incorporated by reference.

To determine the percent identity of two amino acid sequences or of two nucleic acids, the sequences are aligned for optimal comparison purposes, and, if necessary, gaps can be introduced in the first amino acid or nucleic acid sequence for optimal alignment with a second amino or nucleic acid sequence. When a position in the first sequence is occupied by the same amino acid residue or nucleotide as the corresponding position in the second sequence, then the molecules are identical at that position. The percent identity between the two sequences is a function of the number of identical positions shared by the sequences as evaluated, for example, by calculating # of identical positions/total # of positions.times.100. Additional evaluations of the sequence alignment can include a numeric penalty taking into account the number of gaps and size of said gaps necessary to produce an optimal alignment.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

As used herein, the term “molecule” means any compound, including, but not limited to, a small molecule, peptide, protein, sugar, nucleotide, nucleic acid, lipid, etc., and such a compound can be natural or synthetic.

The novel pycobiliproteins, and novel methods of producing the new phycobiliproteins, or novel methods of producing known phycobiliprotiens may be made by assembling various phycobiliprotein production components recombinantly in a cell. Typically this includes 2 or more heterodimeric lyases, and apoprotein, and a bilin reductcase, preferably, a heme oxygenase is also included. Such components include but are not limited to: lyases: CpcE, CpcF, PecE, PecF, CpcS, CpcU, CpcT; bilin reductases: PcyA, PebS, and HY2; apoproteins: CpcA, CpcB, CpeY, CpeS ApcA, ApcB, ApcD, CpeB, CpeA, ApcF, and Cpe2, and preferably heme oxygenase HO1. The genes for these components are generally known and available and may be isolated using standard techniques from publically available plasmids such as but not limited to pPcyA, pPebS, pHY2, pBS414v, pBS405v, pBS405vpecEF, pBS405vpecF, pBS405vpecE, pBS405vcpcEpecF, pBS405vpecECpcF and publically available strains such as Synechocystis sp. PCC 6803, Synechococcus sp. PCC 7002, A. thaliana, and Nostoc sp. PCC 7120, as well as sources such as Genbank (see, for example Accession number AF178757).

In addition, one of skill in the art will recognize many ways of generating alterations in a given nucleic acid sequence. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Berger and Kimmel, Guide to Molecular Cloning Techniques, Methods in Enzymology Volume 152 Academic Press, Inc., San Diego, Calif. (Berger); Sambrook et al. (2001) Molecular Cloning: A Laboratory Manual (3rd ed.) Vol. 1-3, Cold Spring Harbor Laboratory, Cold Spring Harbor Press, N.Y., (Sambrook); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Pirrung et al., U.S. Pat. No. 5,143,854; and Fodor et al., Science, 251:767-77 (1991). Product information from manufacturers of biological reagents and experimental equipment also provide information useful in known biological methods. Such manufacturers include the SIGMA Chemical Company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.), as well as many other commercial sources known to one of skill. Using these techniques, it is possible to substitute at will any nucleotide in a nucleic acid that encodes any phycobilliprotein production component disclosed herein or any amino acid of the same described herein for a predetermined nucleotide or amino acid.

### General Recombinant Methods

The present invention relies on routine techniques in the field of recombinant genetics. Basic texts disclosing the general methods of use in this invention include Sambrook et al., Molecular Cloning: A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (Kb) or base pairs (bp). These are estimates derived from agarose or acrylamide gel electrophoresis, from sequenced nucleic acids, or from published DNA sequences. For proteins, sizes are given in kilodaltons (kDa) or the number of amino acid residues. Proteins sizes are estimated from gel electrophoresis, from automated protein sequencing, from derived amino acid sequences, or from published protein sequences.

Oligonucleotides that are not commercially available can be chemically synthesized according to the solid phase phosphoramidite triester method first described by Beaucage & Caruthers, Tetrahedron Letts., 22:1859-1862 (1981), using an automated synthesizer, as described in Van Devanter et. al., Nucleic Acids Res., 12:6159-6168 (1984). Purification of oligonucleotides is by either native acrylamide gel electrophoresis or by anion-exchange HPLC as described in Pearson & Reanier, J. Chrom., 255:137-149 (1983).

The sequence of cloned genes and synthetic oligonucleotides can be verified after cloning using, e.g., the chain termination method for sequencing double-stranded templates of Wallace et al., Gene, 16:21-26 (1981).

### Cloning Methods for Isolating Nucleic Acids

In general, the nucleic acid sequences encoding pycobiliprotein production components and related nucleic acid sequence homologues are cloned from cDNA and genomic DNA libraries or isolated using amplification techniques with oligonucleotide primers. For example, sequences may be typically isolated from (genomic or cDNA) libraries of cyanobacteria, red algae, and/or cryptomonads, by hybridizing with a nucleic acid probe or polynucleotide, the sequence of which can be derived from publically available plasmids or sequences. Amplification techniques using primers can also be used to amplify and isolate coding sequences from cellular DNA or mRNA. These primers can be used, e.g., to amplify either the full-length sequence or a probe of one to several hundred nucleotides, which is then used to screen a library for a full-length coding sequence.

Nucleic acids encoding pycobilin production components can also be isolated from expression libraries using antibodies as probes. Polymorphic variants, orthologs, and alleles that are substantially identical to the coding region of genes can be isolated using nucleic acid probes and oligonucleotides under stringent hybridization conditions, by screening genomic or cDNA libraries.

cDNA Libraries

Preparation of cDNA libraries can be performed by standard techniques well-known in the art. Well-known cDNA library construction techniques can be found for example, in Sambrook et al., 2001, Molecular Cloning: A Laboratory Manual (3rd ed.); Cold Spring Harbor Laboratory, Cold Spring Harbor, N.Y.

To make a cDNA library, one should choose a source that is rich in phycobiliprotein production components mRNA, e.g., cyabibacteria, red algei, cryptomonads. The mRNA is then made into cDNA using reverse transcriptase, ligated into a recombinant vector using T4 DNA ligase, and transformed into a recombinant bacterial host for propagation, screening and cloning. Methods for making and screening cDNA libraries are well-known (see, e.g., Gubler & Hoffman, Gene, 25:263-269 (1983); Sambrook et al., supra; Ausubel et al., supra).

A phycobiliprotein production components-containing cDNA library constructed in a bacteriophage or plasmid shuttle vector can be screened, for example, with a labeled oligonucleotide probe designed from the nucleic acid sequences disclosed herein and obtained from the sources identified herein. The oligonucleotide probe design can be a partial cDNA encoding phycobiliprotein production components, obtained by specific PCR amplification of phycobiliprotein production components DNA fragments using degenerate oligonucleotide primers based on the amino acid sequence determined from N-terminal amino acid sequencing of phycobiliprotein production components. Alternatively PCR amplification techniques, such as those discussed in detail below, can be used to isolate the phycobiliprotein production components-encoding cDNA.

It will be readily apparent to those skilled in the art that other types of libraries, as well as libraries constructed from other cell types or species types, may be useful for isolating a phycobiliprotein production components-encoding cDNA or a homologue of an phycobiliprotein production components-encoding cDNA. Other types of libraries include, but are not limited to, cDNA and genomic libraries derived from cells or cell lines other than Synechocystis or Nostoc sp., such as cyanobacteria, red algae, and/or cryptomonads or any other such host which may contain phycobiliprotein production components-encoding cDNA.

### Genomic Libraries

For a genomic library, the DNA is extracted from the tissue and either mechanically sheared or enzymatically digested to yield fragments of about 12-20 Kb. The fragments are then separated by gradient centrifugation from undesired sizes and are constructed in bacteriophage λ vectors. These vectors and phage are packaged in vitro. Recombinant phage are analyzed by plaque hybridization as described in Benton & Davis, Science, 196:180-182 (1977). Colony hybridization is carried out as generally described in Grunstein et al., Proc. Natl. Acad. Sci. USA., 72:3961-3965 (1975). See also, Gussow, D. and Clackson, T., Nucl. Acids Res., 17:4000 (1989).

### PCR Amplification

Polymerase chain reaction, or other in vitro amplification methods, may also be useful, for example, in cloning nucleic acid sequences encoding proteins to be expressed; in making nucleic acids to use as probes for detecting the presence of phycobiliprotein production components encoding mRNA in physiological samples; for nucleic acid sequencing, or other purposes (see U.S. Pat. Nos. 4,683,195 and 4,683,202; PCR Protocols. A Guide to Methods and Applications (Innis et al., eds, 1990)). Such methods can be used to PCR amplify phycobiliprotein production components nucleic acid sequences directly from mRNA, or from either genomic or cDNA libraries. Degenerate oligonucleotides can be designed to amplify phycobiliprotein production components homologues using the sequences or sources or plasmids provided herein. Restriction endonuclease sites can be incorporated into the primers. Genes amplified by the PCR reaction can be purified from agarose gels and cloned into an appropriate vector.

PCR techniques include 5′ and/or 3′ RACE techniques, both being capable of generating a full-length cDNA sequence from a suitable cDNA library (Frohman, et al., Proc. Natl. Acad. Sci. USA, 85:8998-9002 (1988)). The strategy involves using specific oligonucleotide primers for PCR amplification of phycobiliprotein production components cDNA. These specific primers are designed through identification of nucleotide sequences either in the cDNA itself, and/or the vector comprising the cDNA.

### Synthetic Nucleic Acid Constructs

Synthetic oligonucleotides can also be used to construct recombinant phycobiliprotein production components genes for use as probes or for expression of protein. This method is performed using a series of overlapping oligonucleotides usually 40-120 bp in length, representing both the sense and non-sense (antisense) strands of the gene. These DNA fragments are then annealed, ligated and cloned. Alternatively, amplification techniques can be used with precise primers to amplify a specific subsequence of the phycobiliprotein production components gene. The specific subsequence is then ligated into an expression vector.

### Expression of Phycobiliprotein Production Components in Prokaryotes and Eukaryotes

The nucleic acids and proteins of the present invention can be expressed in a variety of host cell types, both prokaryotic and eukaryotic. Although some host systems are able to incorporate “naked” nucleic acids devoid of regulatory sequences (e.g., through recombination), generally the nucleic acid must be incorporated into a suitable expression vector to be expressed.

Suitable expression vectors typically comprise regulatory sequences suitable for expression of the nucleic acid in the host cell. These regulatory sequences are necessarily operably linked to the nucleic acid to control its expression. The expression vector may optionally comprise other regulatory, replication or manipulation sequences to aid in the expression and incorporation of the nucleic acid into the expression vector, as required by the particular application being pursued.

For example, to obtain a high level expression of phycobiliprotein production components protein in a prokaryotic system, it is essential to construct expression vectors that contain, at a minimum; a strong promoter to direct transcription, a ribosome-binding site for translational initiation, a transcription/translation terminator, a bacterial replicon, and unique restriction sites in nonessential regions of the plasmid to allow insertion of foreign nucleic acids. Other factors may also be carried on the expression vector, such as selectable and/or scoreable markers, such as an antibiotic resistance gene that is expressed when the expression vector has been constructed and is functioning properly.

### Expression Vectors

Suitable expression vectors for phycobiliprotein production components and related sequences include plasmids, viruses, bacteriophage, integratable DNA fragments, and other vehicles that enable the integration of DNA fragments into the genome of the host. Plasmids are the most commonly used form of vector but all other forms of vectors which serve an equivalent function and which are, or become, known in the art are suitable for use herein. See, e.g., Pouwels, et al. (1985 and Supplements) Cloning Vectors: A Laboratory Manual, Elsevier, N.Y.; Rodriquez, et al. (eds.) Vectors: A Survey of Molecular Cloning Vectors and Their Uses, Buttersworth, Boston, 1988; and Luckow, V. A. and Summers, M. D., BioTechnology, 6:47-55 (1988) all of which are incorporated herein by reference.

Expression vectors used to transform host cells preferably contain DNA sequences to initiate transcription, and sequences to control the translation of the phycobiliprotein production components nucleic acid sequence. These sequences are referred to as regulatory elements. To obtain a high level expression of a cloned gene, such as those cDNAs encoding phycobiliprotein production components, a regulatory element typically included in an expression vector is a strong promoter. Suitable bacterial promoters are well-known in the art and described, e.g., in Sambrook et al., and Ausubel et al., supra; Herskowitz, I. and Hagen, D., Ann. Rev. Genet., 14:399-445 (1980); and Yanofsky, C., J. Bacteriol., 158:1018-1024 (1984).

Exemplary yeast promoters can be found in Hitzeman et al., J. Biol. Chem., 255:12073-12080 (1980); Alber and Kawasaki, J. Mol. Appl. Gen., 1:419-434 (1982); Young et al., in Genetic Engineering of Microorganisms for Chemicals (Hollaender et al, eds.), Plenum Press, New York, 1982; U.S. Pat. No. 4,599,311 and Russell et al., Nature, 304:652-654 (1983). Other exemplary promoter systems are described in McKnight et al., The EMBO J., 4:2093-2099 (1985) (fungal ADH3 promoter); and Vasuvedan et al., FEBS Lett., 311:7-11 (1992) (the insect polyhedrin promoter).

Other regulatory elements that may be incorporated into expression vectors include enhancer elements (see, Enhancers and Eukaryotic Expression, Cold Spring Harbor Press, Cold Spring Harbor, N.Y. 1983), termination sequences (Palmiter et al., op. cit.); and intron splice sequences (Sprague et al, J. Virol., 45:773-781 (1983). Expression vectors may also optionally contain selectable markers, such as the gene coding for dihydrofolate reductase (DHFR) or the Schizosaccharomyces pombe TPI gene (described by Russell, P. R., Gene, 40:125-130 (1985)). The cloning vector containing the regulatory elements is cleaved using restriction enzymes and adjusted in size as necessary or desirable and ligated with sequences encoding the phycobiliprotein production components proteins by means well-known in the art.

### Host Cells Suitable for Phycobiliprotein Production

Phycobiliprotein production components can be expressed in any host cell capable of accepting and expressing a DNA construct or expression vector of the invention. Suitable host cells include bacteria, yeast, fungi and higher eukaryotic cells, such as plant and mammalian cells. Exemplary bacterial host cells include gram-positive bacteria (Palva et al., Gene, 22:229-235 (1983); Mosbach et al., Nature, 302:543-545 (1983), and gram-negative bacteria such as Echerichia coli (cf. Sambrook et al., supra). Examples of suitable yeast cells include Pichia pastoris, Saccharomyces sp. or Schizosaccharomyces sp. Other suitable fungal hosts include Aspergillus sp., Neurospora sp., Fusarium sp. or Trichoderma sp., in particular strains of A. oryzae, A. nidulans or A. niger.

Higher eukaryotic cells grown in tissue culture are often the preferred host cells for expression of the heterologous proteins such as the phycobiliprotiens disclosed herein. In principle, any higher eukaryotic tissue culture cell line is workable, e.g., insect baculovirus expression systems, whether from an invertebrate or vertebrate source. Mammalian cells are particularly preferred. Examples of suitable mammalian cell lines include He—La cells, Chinese hamster ovary (CHO) cell lines, baby rat kidney (BRK) cell lines, insect cell lines, bird cell lines, and monkey (COS) cell lines.

### Transfection of Host Cells

Standard transfection methods are used to introduce the DNA constructs and expression vectors of the present invention to host cells. Transformation of both eukaryotic and prokaryotic cells are performed according to standard techniques (see, e.g., Morrison, J. Bact., 132:349-351 (1977); Clark-Curtiss & Curtiss, Methods in Enzymology, 101:347-362 (Wu et al., eds, 1983). Transformed host cells usually express phycobiliprotein production components protein or its fragments, but for purposes of cloning, amplifying, and manipulating its DNA do not need to express the protein. This invention further contemplates culturing transformed cells in a nutrient medium, thus permitting phycobiliprotein production components protein to accumulate in the culture. The proteins can be recovered from the cells or from the culture medium by standard protein purification techniques described herein.

Methods for transforming bacterial cells are described in Sambrook et al., and Ausubel et al., supra. Yeast cell transformation with heterologous DNA is described, e.g. in U.S. Pat. No. 4,599,311, U.S. Pat. No. 4,931,373, U.S. Pat. Nos. 4,870,008, 5,037,743, and U.S. Pat. No. 4,845,075. Methods of transfecting mammalian cells are described in e.g. Kaufman and Sharp, J. Mol. Biol., 159:601-621 (1982); Southern and Berg, J. Mol. Appl. Genet., 1:327-341 (1982); Loyter et al., Proc. Natl. Acad. Sci. USA, 79:422-426 (1982); Wigler et al., Cell, 14:725 (1978); Corsaro and Pearson, Somatic Cell Genetics, 7:603 (1981), Graham and van der Eb, Virology, 52:456 (1973); and Neumann et al., EMBO J., 1:841-845 (1982).

### Identifying an Isolated Nucleic Acid Encoding Phycobiliprotein Production Components.

The nucleic acids and proteins encoding phycobiliportein components for procution of the novel proteins of the invention are detected, confirmed and quantified by any of a number of means well-known to those of skill in the art. The unique quality of the expressed proteins here is that they fluoresce, a property that can be readily and easily observed. Fluorescence assays for the expressed proteins are described in detail below. Other general methods for detecting both nucleic acids and corresponding proteins include analytic biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like.

The detection of phycobiliprotein production component nucleic acids can also be accomplished by Southern analysis (Southern et al., J. Mol. Biol., 98:503 (1975)), Northern analysis, gel electrophoresis, PCR, radiolabeling, scintillation counting, and affinity chromatography. In performing nucleic acid hybridization techniques, the format is not critical. Additional formats include sandwich assays and competition or displacement assays. Hybridization techniques are generally described in “Nucleic Acid Hybridization, A Practical Approach,” Ed. Hames, B. D. and Higgins, S. J., IRL Press, 1985; Gall and Pardue, Proc. Natl. Acad. Sci. USA, 63:378-383 (1969); and John et al., Nature, 223:582-587 (1969). Sandwich assays, for example, are commercially useful hybridization assays for detecting or isolating nucleic acid sequences. Such assays utilize a “capture” nucleic acid covalently immobilized to a solid support and labeled “signal” nucleic acid in solution. The clinical sample will provide the target nucleic acid. The “capture” nucleic acid and “signal” nucleic acid probe hybridize with the target nucleic acid to form a “sandwich” hybridization complex. To be effective, the signal nucleic acid cannot hybridize with the capture nucleic acid.

Labeled signal nucleic acids, whether those described herein or others known in the art are used to detect hybridization. Complementary nucleic acids or signal nucleic acids may be labeled by any one of several methods typically used to detect the presence of hybridized polynucleotides, and discussed in detail below.

An alternative means for determining the level of expression of the phycobiliprotein production component genes is in situ hybridization as described in Angerer et al., Methods Enzymol., 152:649-660 (1987). In an in situ hybridization assay cells are fixed to a solid support, typically a glass slide. If DNA is to be probed, the cells are denatured with heat or alkali. The cells are then contacted with a hybridization solution at a moderate temperature to permit annealing of phycobiliprotein production components-specific probes that are labeled. The probes are preferably labeled with radioisotopes or fluorescent reporters.

The sequence of the cloned genes and synthetic oligonucleotides can be verified using the chemical degradation method of A. M. Maxam et al., Methods in Enzymology, 65:499-560 (1980). The sequence can be confirmed after the assembly of the oligonucleotide fragments into the double-stranded DNA sequence using the method of Maxam and Gilbert, supra, or the chain termination method for sequencing double-stranded templates of R. B. Wallace et al., Gene, 16:21-26 (1981). DNA sequencing may also be performed by the PCR-assisted fluorescent terminator method (ReadyReaction DyeDeoxy Terminator Cycle Sequencing Kit, ABI, Columbia, Md.) according to the manufacturer's instructions, using the ABI Model 373A DNA Sequencing System. Sequencing data is analyzed using the commercially available Sequencher program (Gene Codes, Gene Codes, Ann Arbor, Mich.).

### Constructing Variant Phycobiliprotein Production Components

Mutant Phycobiliprotein Production Components

As will be readily apparent to those in the art, to describe a fluorescent protein of the present invention, it is not necessary to provide the entire sequence of any particular phycobiliprotein production components. A functional phycobiliprotein production component is characterized by a functional phycobiliprotein production component chromophore, as defined herein. In order for the chromophore to be functional however, an phycobiliprotein must possess a tertiary structure that provides the chromophore with a suitable environment, particularly support of the correct chromophore orientation, to allow the chromophore to fluoresce. Consequently, minor deletions at either end of the protein sequence are expected to have little or no impact on the fluorescence spectrum of the protein. Moreover, modifications to the primary amino acid sequence that do not perturb the tertiary structure of the protein, including substitution, deletion and insertion of amino acids or chemical modification of amino acid residues of the protein, are expected to modify the absorption and/or emission spectrum of the phycobiliprotein production components, but not extinguish fluorescence. Therefore, both mutant and wild-type phycobiliprotein sequences are contemplated, as manifested not only in the complete polypeptide and oligonucleotide sequences discussed herein, but also functionally equivalent portions and mutations thereof (i.e., portions of the polypeptide sequences which exhibit the desired fluorescence properties and oligonucleotide sequences encoding these polypeptide sequences).

A functionally equivalent mutation includes mutations with neutral (or minor) effects on the fluorescent properties of the claimed proteins and mutations with more dramatic effects, including large shifts in absorbance, emission and/or excitation spectra. Whereas some amino acids of the chromophore itself (position 60-64) are obviously important, the locations of neutral mutations suggest that other amino acids of the claimed proteins are less critical to fluorescence. Other mutations encompassed by the invention modify spectroscopic properties of the protein without altering the amino acid sequence giving rise to the phycobiliprotein production components chromophore.

Necessarily, mutations outside the chromophore that modify in character, but do not extinguish completely, the fluorescence of a phycobiliprotein production components protein of the present invention are included in the claimed invention.

In addition, various types of fusion sequences that lengthen the resultant protein or serve some functional purpose in the preparation or purification of the protein are also routine and contemplated as within the scope of the present invention. As an example, it is common practice to add amino acid sequences including a polyhistidine tag to facilitate purification of product proteins, as detailed below herein, or “fuse” two molecules through covalent attachment with or without an intervening linker region. Such modifications generally do not significantly alter the salient properties of the molecules making up the fusion protein. Phycobiliprotein production components modified in this manner are therefore also contemplated as within the scope of the present invention.

The DNA sequence coding for phycobiliprotein production components may also be modified by other means such as by conventional chemical mutagenesis or by insertion, deletion or substitution of one or more nucleotides in the sequence, either as random or site-directed mutagenesis. It is expected that such mutants will exhibit altered optical properties or altered heat stability.

De Novo Synthesis

The DNA construct of the invention encoding phycobiliprotein production components, modified phycobiliprotein production components or hybrid polypeptide may be prepared synthetically by established standard methods, e.g. the phosphoramidite method described by Beaucage and Caruthers, Tetrahedron Letters, 22:1859-1869 (1981), or the method described by Matthes et al., EMBO J., 3:801-805 (1984). According to the phosphoramidite method, oligonucleotides are synthesized, e.g. in an automatic DNA synthesizer, purified, annealed, ligated and cloned in suitable vectors. Moreover, the protein sequence of a number of phycobiliprotein production components has been deduced. Using the methods described above, it is within the skill of one in the art to synthesize novel phycobiliprotein production components through de novo synthesis of a polynucleotide that, for example, substantially encodes the same but with the substitutions.

Furthermore, the DNA construct may be of mixed synthetic and genomic, mixed synthetic and cDNA or mixed genomic and cDNA origin, prepared by ligating fragments of synthetic, genomic or cDNA origin (as appropriate) in accordance with standard techniques (cf. Sambrook et al., Molecular Cloning: A Laboratory Manual, 3rd, 2001, Cold Spring Harbor Laboratory, New York, USA). The fragments corresponding to various parts of the entire DNA construct, including the sequence of the substituting phycobiliprotein production components chromophore, can optionally be from any source including different phycobiliprotein production components's, and combined to form novel phycobiliprotein production components. Alternatively, a phycobiliprotein chromophore may be “swapped” into different polypeptides or fragments. See, e.g., Cunningham, et al., Science, 243:1330-1336 (1989); and O'Dowd, et al., J. Biol. Chem., 263:15985-15992 (1988), each of which is incorporated herein by reference. Thus, new chimeric phycobiliprotein production components's exhibiting new combinations of fluorescent properties will result from the functional linkage of the phycobiliprotein production components chromophore to alternative heterologous protein scaffolds.

Site-Directed Mutagenesis

Site-directed mutagenesis may be used to prepare further variants of nucleic acids encoding phycobiliprotein production components. Site-directed mutagenesis is a technique useful in the preparation of individual peptides, or biologically functional equivalent proteins or peptides, through specific mutagenesis of the underlying DNA. The technique further provides a ready ability to prepare and test sequence variants by introducing one or more nucleotide sequence changes into the DNA.

The technique of site-directed mutagenesis is generally well-known in the art as exemplified by publications (Adelman et al., DNA, 2:183, (1983)). As will be appreciated, the technique typically employs a phage vector that exists in both a single-stranded and double-stranded form. Typical vectors useful in site-directed mutagenesis include vectors such as the M13 phage (Messing et al., Third Cleveland Symposium on Macromolecules and Recombinant DNA, Ed: A. Walton, Elsevier, Amsterdam, (1981)). These phages are readily commercially available and their use is generally well-known to those skilled in the art. Double-stranded plasmids are also routinely employed in site-directed mutagenesis, which eliminates the step of transferring the gene of interest from a plasmid to a phage.

In general, site-directed mutagenesis in accordance herewith is performed by first obtaining a single-stranded nucleic acid that includes within its sequence the coding sequence for an phycobiliprotein production component or phycobiliprotein production component peptide. For example, an oligonucleotide that is generally complimentary with the region of the phycobiliprotein production comprising the chromophore but bearing nucleotide substitutions required to encode in frame the phycobiliprotein production component chromophore pentapeptide at the position of the original phycobiliprotein production components chromophore is generated. Such oligonucleotides can be generated for example by the de novo (phosphoramidite) synthesis techniques noted above. This oligonucleotide is then annealed with the single-stranded nucleic acid comprising the sequence for the phycobiliprotein production components, and subjected to DNA polymerizing enzymes such as E. coli polymerase I Klenow fragment, in order to complete the synthesis of the mutation-bearing strand. A heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement. Typically, a primer of about 17 to 25 nucleotides in length is preferred, with about 5 to 10 residues on both sides of the junction of the sequence being altered. Suitable techniques are also described in U.S. Pat. No. 4,888,286, incorporated herein by reference.

The preparation of sequence variants of a phycobiliprotein production component using site-directed mutagenesis is provided as a means of producing novel, potentially useful phycobiliprotein production components species and is not meant to be limiting as there are other ways in which sequence variants of phycobiliprotein production components genes may be obtained. For example, recombinant vectors comprising a nucleic acid encoding phycobiliprotein production components may be treated with mutagenic agents to obtain sequence variants (see, e.g., the method described by Eichenlaub, J. Bacteriol, 138:559-566 (1979)).

Although the foregoing methods are suitable for use in mutagenesis, the use of the polymerase chain reaction (PCR) is generally now preferred. Briefly, the phycobiliprotein chromophore is replaced by the phycobiliprotein chromophore by amplifying a nucleic acid encoding the phycobiliprotein chromophore with primers generally specific for the phycobiliprotein nucleotide sequence, but were at least one of the primers comprises nucleotide substitutions creating an in frame coding sequence for the phycobiliprotein chromophore pentapeptide.

Resulting reaction products should be examined by e.g., restriction mapping, electrophoresis and/or automated nucleotide sequencing to confirm the desired product is obtained. The synthesized nucleic acid containing the phycobiliprotein production components chromophore pentapeptide can then be expressed in a suitable system, as described above.

Purification of Phycobiliprotein Production Components or Generated Phycobiliproteins

Culture and purification techniques are those standard in the art. (cf., Colley et al., J. Biol. Chem., 264:17619-17622 (1989), and Guide to Protein Purification, in Vol. 182 of Methods in Enzymology (Deutscher ed., 1990), Morrison, D. A., J. Bact., 132:349-351 (1977), or by Clark-Curtiss et al., Methods in Enzymology, 101:347-362 (1983), eds. R. Wu et al., Academic Press, New York. (for suitable media, see the catalogues of the American Type Culture Collection)). Additional isolation techniques are described in detail in the following sections.

Either naturally occurring or recombinant phycobiliprotein production components or resultant assembled phycobiliprotiens can be purified for use in functional assays. Naturally occurring phycobiliprotein production components can be purified, e.g., from Cerianthus sp., or any other source of an phycobiliprotein production components homologue. Recombinant phycobiliproteins and/or their production components can be purified from any suitable expression system.

Phycobiliprotein or their production components may be purified to substantial purity by standard techniques, including column chromatography, immunopurification methods, electrophoresis, centrifugation, crystallization, isoelectric focusing and others (see, e.g., Scopes, Protein Purification: Principles and Practice (1982); U.S. Pat. No. 4,673,641; Ausubel, et al. (1987 and periodic supplements) Current Protocols in Molecular Biology; Deutscher (1990) “Guide to Protein Purification” in Methods in Enzymology vol. 182, and other volumes in this series; and manufacturers' literature on use of protein purification products, e.g., Pharmacia, Piscataway, N.J., or Bio-Rad, Richmond, Calif.; and Sambrook et al., supra).

A number of procedures can be employed when recombinant phycobiliproteins or their production components are being purified. For example, proteins having established molecular adhesion properties can be reversibly fused to the phycobiliprotein production components. With the appropriate ligand, phycobiliprotein production components can be selectively adsorbed to a purification column and then freed from the column in a relatively pure form. The fused protein is then removed by enzymatic activity. Finally phycobiliprotein production components could be purified using immunoaffinity columns.

Purification from Recombinant Bacteria

Recombinant proteins are expressed by transformed bacteria in large amounts, typically after promoter induction; but expression can be constitutive. Promoter induction with IPTG is one example of an inducible promoter system. Bacteria are grown according to standard procedures in the art, as noted above. Fresh or frozen bacteria cells are used for isolation of protein.

Proteins expressed in bacteria may form insoluble aggregates (“inclusion bodies”). Several protocols are suitable for purification of phycobiliproteins or their production components from inclusion bodies. For example, purification of inclusion bodies typically involves the extraction, separation and/or purification of inclusion bodies by disruption of bacterial cells, e.g., by incubation in a buffer of 50 mM Tris/HCl pH 7.5, 50 mM NaCl, 5 mM MgCl₂, 1 mM DTT, 0.1 mM ATP, and 1 mM PMSF. The cell suspension can be lysed using 2-3 passages through a French Press, homogenized using a Polytron (Brinkmann Instruments, Inc.) or sonicated on ice. Alternate methods of lysing bacteria are apparent to those of skill in the art (see, e.g., Sambrook et al., supra; Ausubel et al., supra). phycobiliprotein production components in the lysate can then be purified using standard techniques (see, e.g., Colley et al., J. Biol. Chem., 264:17619-17622 (1989); Guide to Protein Purification, in Methods in Enzymology, vol. 182 (Deutscher, ed., 1990)).

If necessary, the inclusion bodies are solubilized, and the lysed cell suspension is typically centrifuged to remove unwanted insoluble matter. phycobiliprotein production components comprising the inclusion bodies may be renatured by dilution or dialysis with a compatible buffer. Suitable solvents include, but are not limited to urea (from about 4 M to about 10 M), formamide (at least about 80%, volume/volume basis), and guanidine hydrochloride (from about 4 M to about 8 M). Some solvents, which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is reversible and renaturation may occur upon removal (by dialysis, for example) or dilution of the denaturant, allowing re-formation of immunologically and/or biologically active protein. Other suitable buffers are known to those skilled in the art. It is important to note that the phycobiliprotein production components structure is extremely stable once formed, and readily folds in the correct conformation when denaturant is removed.

Alternatively, it is possible to purify phycobiliproteins or their production components from bacteria periplasm. When phycobiliproteins or their production components are exported into the periplasm of the bacteria, the periplasmic fraction of the bacteria can be isolated by cold osmotic shock in addition to other methods known to skill in the art. To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well-known to those of skill in the art.

### Standard Purification Techniques

Gel Filtration

The molecular weight of phycobiliproteins or their production components (e.g., approximately 25.2 kDa) can be used to isolate it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut-off greater than the molecular weight of the protein of interest. The recombinant protein will pass through the membrane into the filtrate. The filtrate can then be chromatographed as described below.

Exchange Chromatography

Phycobiliproteins or their production components can also be separated from other proteins on the basis of size, net surface charge, hydrophobicity, and affinity for ligands. In addition, antibodies raised against proteins can be conjugated to column matrices and the proteins immunopurified. All of these methods are well-known in the art. It will be apparent to one of skill that chromatographic techniques can be performed at any scale and using equipment from many different manufacturers (e.g., Pharmacia Biotech).

Tagging Techniques

Purification segments, or “affinity tags” can be fused to appropriate portions of the receptor to assist in isolation and production. For example, the FLAG sequence, or a functional equivalent, can be fused to the protein via a protease-removable sequence, allowing the FLAG sequence to be recognized by an affinity reagent, and the purified protein subjected to protease digestion to remove the extension. Many other equivalent segments exist, e.g., poly-histidine segments possessing affinity for heavy metal column reagents. See, e.g., Hochuli, Chemische Industrie, 12:69-70 (1989); Hochuli, Genetic Engineering, Principle and Methods, 12:87-98 (1990), Plenum Press, N.Y.; and Crowe, et al. (1992) OIAexpress: The High Level Expression & Protein Purification System, QIAGEN, Inc. Chatsworth, Calif.; which are incorporated herein by reference.

His-Tag

The phycobiliproteins or their production components construct can also contain a string of histidine residues, incorporated at the amino or carboxyl terminal of the phycobiliprotein production components. The polyhistidine tag allows convenient isolation of the protein in a single step by nickel-chelate chromatography. When a protein that has been “his-tagged” is placed on the nickel column, the histidine residues form a chelate complex with the nickel bound to the column, immobilizing the tagged protein. Contaminating components of the solution comprising the tagged protein can be washed away prior to elution of the tagged protein with a suitable competing chelator, typically imidazole.

The polyhistidine tag can be added to the protein through the use of peptide linkers as described in detail below. Alternatively, the tag can be linked to phycobiliprotein production components by appending a nucleic acid encoding the tag onto the coding region of phycobiliprotein production components, the resulting construct being incorporated into a suitable expression vector that is subsequently used to transform an appropriate host cell.

Epitope Tagging

Epitope tags are another useful sequence that can be included in the phycobiliproteins or their production components construct. The epitope tag can consist of an amino acid sequence that allows affinity purification of the activated protein (e.g., on immunoaffinity or chelating matrices). Thus, by including an epitope tag on the activation construct, all of the activated proteins from an activation library can be purified. By purifying the activated proteins away from other cellular and media proteins, screening for novel proteins and enzyme activities can be facilitated. In some instances, it may be desirable to remove the epitope tag following purification of the activated protein. This can be accomplished by including a protease recognition sequence (e.g., Factor Xa or enterokinase cleavage site) downstream from the epitope tag on the activation construct. Incubation of the purified, activated protein(s) with the appropriate protease will release the epitope tag from the proteins(s).

In libraries in which an epitope tag sequence is located in the phycobiliproteins or their production components construct, all of the phycobiliproteins or production components can be purified away from all other cellular and media components using affinity purification. In addition to purifying the tagged phycobiliprotein production components protein, this method also concentrates the protein sample.

PAGE/Blotting

Phycobiliproteins or their production components of the present invention can be purified using native polyacrylamide gel electrophoresis. Briefly, the technique involves preparing a polyacrylamide gel slab by mixing appropriate amounts of acrylamide and bis-acrylamide in a basic buffer solution, typically Tris-HCl based, and allowing the mixture to polymerize between a pair of parallel glass plates uniformly-spaced. By modifying the amount of acrylamide added to the mixture, slabs can be optimized for separation of proteins in particular molecular weight ranges. In the case of phycobiliprotein production components, a preferred acrylamide content for the gel would be between 6% and 15%, more preferably between 8% and 12%. The gel is normally loaded and run in the vertical position, with protein resolution resulting by a sieving action of the gel as the proteins are driven through the gel matrix by an electrical current applied across the gel slab. (see Schagger et al., Anal. Biochem., 166:368-379 (1987)).

Once protein resolution is complete, the bands containing phycobiliproteins and production components are easily recognized as they fluoresce when exposed to a light comprising the appropriate wavelength. The band(s) containing the proteins are excised from the gel, and the resulting gel slices placed in a dialysis sack with the appropriate molecular weight cut-off and containing a buffer solution with a pH value preferably between 7 and 9, more preferably between 7.5 and 8.5. The sack is placed on a flat bed electrophoresis unit parallel to the direction of the current. The electrophoresis unit is filled with the same buffer solution placed in the dialysis sack. The electrophoresis unit is run for several hours, preferably overnight, at a low voltage of between 5 and 50 volts, more preferably between 15 and 30 volts (the actual voltage applied depends upon the application, particularly the composition of the buffer solution used in the apparatus).

By subjecting the gel slice containing the phycobiliproteins or their production components protein to the low voltage and current of the flat bed electrophoresis apparatus, the proteins are driven out of the gel slice and into the buffer solution of the dialysis sack. Once electrophoresis is complete, the vacant gel slices can be removed, and the protein in the buffer solution concentrated using any one of the variety of concentration methods known in the art. All chemicals and apparatus used in the methods are described in available scientific literature and commonly available through scientific catalogs. (see, e.g., Scopes, Protein Purification: Principles and Practice (1982); Ausubel, et al. (1987 and periodic supplements); Current Protocols in Molecular Biology; Deutscher (1990) “Guide to Protein Purification” in Methods in Enzymology vol. 182, and other volumes in this series; and manufacturers' literature on use of protein purification products, e.g., Pharmacia, Piscataway, N.J., or Bio-Rad, Richmond, Calif.; and Sambrook et al., supra).

Alternatively, proteins resolved by the vertical gel electrophoresis method can be transferred, using Western blotting techniques commonly known in the art, to nylon or PVDF membranes, or the like. Portions of the membranes containing phycobiliprotein production components can then be isolated using the fluorescent properties of the protein for identification. The isolated membrane portions can then be analyzed to characterize the fluorescent protein, e.g., Western blotting or N-terminal amino acid sequencing, as described herein. See Mozdzanowsky et al., Electrophoresis, 13:59-64 (1992).

Isoelectric Focusing

Isoelectric focusing can be performed in both analytical and preparative applications. The principles of separation are the same regardless of the application, only the quantities of proteins, pH range and quantity of carrier ampholytes employed, and the sample capacity of the apparatus differ between applications.

IEF takes place in a pH gradient and is limited to molecules that can be either positively or negatively charged (amphoteric molecules), like proteins, enzymes and peptides. Protein separation occurs in a pH gradient formed by special amphoteric buffers (ampholytes) having high buffer capacities at their pI (isoelectric point). The pH gradient is produced by an electric field. Before an electric field is applied the gel has a uniform pH-value and almost all the carrier ampholytes are charged. When an electric field is applied, negatively charged ampholytes move towards the anode, the positively charged ampholytes to the cathode. The carrier ampholytes align themselves between the cathode and the anode according to their pI. Hundreds or thousands of carrier ampholytes aligned in this manner form partially overlapping distributions, establishing the pH gradient. As there are no other ionic species in the system, each carrier ampholyte must act as counter ion to other carrier ampholytes consequently each position in the pH gradient will have a unique chemical composition. Electrical conductance and buffer capacity will therefore vary over the pH gradient. Regions with low buffer capacity are more prone to distortion. In preparative experiments with protein loads, buffering capacity form the proteins may affect the pH gradient.

A large number of carrier ampholyte mixtures are available giving different pH gradients. Many can also be obtained in pre-cast gels ready to use. The optimal pH gradient will depend on the purpose of the experiment. For screening purposes, a broad range interval (pH 3-10 or similar) should be used. A narrow pH range interval is useful for careful pI determinations or when analyzing proteins with very similar pI points. Generally, one should not use a narrower gradient than necessary because the shallower gradient will lead to longer focusing times and more diffuse bands. When choosing pH gradient one should be aware that the interval stated by the manufacturer can only be an approximation. The exact gradient obtained depends on many factors such as choice of electrolyte solutions, gradient medium (PAA or agarose), focusing time etc.

In polyacrylamide gels, pore size can be accurately controlled by the total acrylamide-concentration and degree of cross-linking (relationship between acrylamide and bis-acrylamide). When cross-linking is kept constant and total concentration increases pore size will decrease (and diffusion is reduced). Gel solution is made from appropriate amounts of acrylamide (about 5%), ampholyte (about 2%), double distilled H₂O, and riboflavin 5′ phosphate (for photopolymerization). Gels (approx 250×120×1 mm) are mold between two glass-plates and polymerized overnight in UV-light (requires a pH in the solution >5-6).

Once the pH gradient has formed in the gel, samples are applied cathodically. The charged components in the samples will migrate according to their net charge, until they encounter a region of the gel were the pH is equal to the sample components pI. At this equivalence point, the protein is uncharged and stops migrating. Should the sample component diffuse into an adjacent pH-environment, it will rapidly acquire a charge and move back to the position corresponding to its pI. Most proteins have a pI in the range of 5 to 8.5, with the phycobiliprotein production components formed by the polypeptide of SEQ ID NO: 2 has a pI of between 6 and 8. Further explanation of IEF techniques may be found in Scopes, Protein Purification: Principles and Practice (1982); Current Protocols in Molecular Biology; Deutscher (1990) “Guide to Protein Purification” in Methods in Enzymology vol 182, and other volumes in this series; and manufacturers' literature on use of protein purification products, e.g., Bio-Rad, Richmond, Calif.; and Sambrook et al., supra).

The Rotofor™, produced by the Bio-Rad Corporation, is an example of an apparatus suitable for the purification of relatively large quantities of proteins by IEF. By way of example, the phycobiliproteins formed from the methods of the invention can be purified using the Bio-Rad Rotofor™ apparatus in a manner described by the manufacturer. Briefly, after a gradient-forming pre-run of the system with 18 ml 2% ampholyte (pH 3 to 10) for 1 hour, phycobiliprotein production components is injected into the system. After focusing the system for several hours, phycobiliproteins are enriched in fractions having a pH between 6 and 8. Phycobiliproteins are easily identified by illumination with ultra violet light.

Analyzing Phycobiliproteins

Labels for Proteins and Nucleic Acids

The particular label or detectable group used in the assay is not a critical aspect of the invention, as long as it does not significantly interfere with the specific binding of the antibody or protein used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™); fluorescent dyes and techniques capable of monitoring the change in fluorescent intensity, wavelength shift, or fluorescent polarization (e.g., fluorescein isothiocyanate, Texas red, rhodamine, and the like); radiolabels (e.g., ³H, ¹²⁵I, ³⁵S, ¹⁴C, or ³²P); enzymes (e.g., horse radish peroxidase, alkaline phosphatase and others commonly used in an ELISA); and colorimetric labels such as colloidal gold or colored glass or plastic beads (e.g., polystyrene, polypropylene, latex, etc.). For exemplary methods for incorporating such labels, see U.S. Pat. Nos. 3,940,475; 3,817,837; 3,850,752; 3,939,350; 3,996,345; 4,277,437; 4,275,149; and 4,366,241.

The label may be coupled directly or indirectly to the desired component of the assay according to methods well-known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), that is either inherently detectable or covalently bound to a signal system, such as a detectable enzyme, a fluorescent compound, or a chemiluminescent compound. The ligands and their targets can be used in any suitable combination with antibodies that recognize phycobiliprotein production components, or secondary antibodies that recognize anti-phycobiliprotein production components antibodies. Other possibilities for indirect labeling include biotinylation of one constituent followed by binding to avidin coupled to one of the above label groups.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazined-iones, e.g., luminol. For a review of various labeling or signal producing systems that may be used, see, U.S. Pat. No. 4,391,904.

Means of detecting labels are well-known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge-coupled devices (CCDs) or photomultipliers and the like. Similarly, enzymatic labels may be detected by providing the appropriate substrates for the enzyme and detecting the resulting reaction product. Finally, simple calorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Synthesis of Phycobiliproteins

To make the bilin, the cell generally produces a heme precursor which is subject to a heme oxygenase to form a biliverdin. The heme oxygenase may be native or recombinant, such as a recombinantly expressed HO1 from Synechocystis sp. PCC6803. The biliverdin is generally further subject to the recombinant bilin reductase and may be further subject to additional enzymes of the cell such as additional reductases, to form the required bilin, which is joined to the required phycobiliprotein domain by the required lyase. However, because biliverdin can form fluorescent adducts with phycobiliproteins and because the tetrapyrrole moiety may be subject to further modification, e.g. isomerization, the term “bilin”, as used herein, encompasses tetrapyrroles that combine or are already combined with a phycobiliprotein domain to form a fluorescent adduct wherein the tetrapyrrole moiety provides light harvesting, energy transfer functionality to the adduct. Hence, the term encompasses isomeric precursors of the tetrapyrrole moieties of the functional adducts.

A wide variety of recombinant reductases, phycobiliprotein domains, and lyases may be used, and in a wide variety of combinations to obtain a wide variety of recombinant fluorescent phycobiliproteins.

In particular embodiments, the invention utilizes 3Z-phycocyanobilin:ferredoxin oxidoreductase (PcyA) or 3Z-phycoerythrobilin:ferredoxin oxidoreductase (PebS, PebA, or PebB). In addition to PcyA, which converts biliverdin to PCB, PebS, which converts biliverdin to 3Z-phycoerythrobilin, and HY2, which converts biliverdin to phytochromobilin, have been reported. Similarly, any phycobiliprotein bilin lyase which yields the requisite recombinant fluorescent phycobiliprotein adduct will suffice; noting that the bilin moiety may be subject to further modification to effect or alter fluorescent properties of the ultimate fluorescent phycobiliprotein.

The bilin lyases CpcE and CpcF are exemplified in detail below. Suitable bilin lyases with specificities different from CpcE and CpcF include PecE and PecF, which catalyze the addition of PCB to phycoerythrocyanin apo-α subunit and the isomerization of the bound bilin to phycobiliviolin (Jung, et al. (1995) J. Biol. Chem. 270, 12877-12884; Zhao, et al. (2000) FEBS Lett. 469, 9-13). CpeY plus CpeZ have been reported to catalyze the addition of phycoerythrobilin to one of the bilin attachment sites on the α subunit of C-phycoerythrin (Kahn, et al., (1997) J. Bacteriol. 179, 998-1006). The lyase may provide any required isomerase activity, or such activity may be provided by an independent isomerase, which may be endogenous or recombinant.

The required phycobiliprotein domain is similarly limited only by the functional requirement that it be combinable by the lyase with the bilin to form the required fluorescent phycobiliprotein. The phycobiliprotein domain may be expressed independently, or as a fusion protein with a heterologous protein domain (i.e. not naturally fused to the phycobiliprotein domain). Any phycobiliprotein domain having the requisite functionality may be used and these may be derived from natural, semisynthetic or synthetic sequences. A wide variety of natural phycobiliproteins are known in the art, e.g. Apt and Grossman, 1995, J Mol Biol 248, 79-96, including proteins derived from many cyanobacteria, rhodophytes (red algae) and cryptomonads, etc. (see, e.g. Glazer, 1994, J Appl Phycol 6, 105-112; Glazer et al. 1995, Photosynth Res 46, 93-105), particularly phycoerythrins, phycocyanins, and allophycocyanins. In addition, a wide variety of methods are known for modifying such natural sequences to generate semi-synthetics, e.g. Glazer, 1994, supra, describes phycobiliproteins having non-natural, predetermined bilin compositions, and Toole et al., 1998, Mol Micro 30, 475-486 describes recombinations of phycobiliprotein deletion mutants. Finally, known phycobiliprotein structure-function relationships (e.g. Anderson et al., 1998, Mol Micro 30, 467-474) are exploited to generate synthetic sequence analogs using conventional methods.

The phycobiliprotein domain confers fluorescence on the fusion protein, preferably providing fluorescence quantum yield and molar extinction coefficients at least 1%, preferably at least 10%, more preferably at least 50%, more preferably at least 75%, more preferably at least 90% and most preferably substantially equivalent to that of a corresponding unfused phycobiliprotein, measured as described herein. Preferred domains provide extinction coefficients of at least 100K, preferably at least 300K, more preferably at least 1M and/or quantum yields of at least 0.25, preferably at least 0.5, more preferably at least 0.6, as described herein. In other preferred aspects of this embodiment, the fluorescence emission spectrum of the fusion protein is substantially equivalent to that of a corresponding unfused phycobiliprotein.

The phycobiliprotein domain comprises one or more bilins, preferably a plurality of functional bilins contributing to the visible absorption spectrum of the phycobiliprotein domain and fusion protein, preferably natural bilins. The bilins are generally covalently coupled to the phycobiliprotein domain through cysteine thioether linkages, preferably at natural bilin attachment sites. Hence, the phycobiliprotein domain of the fusion protein provides a substrate for enzymatic bilin addition, which may provide natural or non-natural bound bilin distribution, preferably a substrate for enzymes which naturally modify a corresponding natural phycobiliprotein.

The fusion proteins may comprise additional components as desired, which may provide modules of functionalities, such as affinity handles, dimer- or oligomerization domains, stabilization domains, specificity domains, signaling domains, etc., apart from any such functionality/ies provided by the displayed domain. For example, for constructs to be used as fluorescent labels, introduction of GCN oligomerization domains enhances both the spectroscopic value (more chromophores) and binding affinity (more sites for intermolecular interaction).

Accordingly, the heterologous protein domain may be a target protein to be labeled or traced, a second labeled domain such as a second fluorescent domain, which may be provided by a different phycobiliprotein domain, a phytochrome domain, a GFP domain, etc., etc. In a particular embodiment, the heterologous protein domain is fluorescent and spectroscopically distinguishable from the fluorescent, first holo-phycobiliprotein domain, particularly wherein the fusion protein provides fluorescence resonance energy transfer between the fluorescent first phycobiliprotein domain and the heterologous protein domain.

In other particular embodiments, the fusion protein comprises a specific binding moiety comprising at least one of a specific binding pair, such as a receptor-ligand pair, e.g. an immunoglobulin antigen-binding domain or antigenic domain, a lectin saccharide-binding domain or glycosylated or glycosylatable domain, an avidin or streptavidin biotin-binding domain or biotinylated or biotinylatable (i.e. providing a substrate for enzymatic biotinylation) domain, etc. In a particular embodiment, the fusion protein comprises a biotinylated or biotinylatable domain, which is preferably biotinylated in the expression system (e.g. cell) selected for expression of the fusion protein. A wide variety of synthetic, semi-synthetic and natural such domains are known in the art, see e.g., Schatz et al. 1993, Bio/Technology 11, 1138-1143; Tatsumi et al., 1996, Anal Biochem 243, 176-180; Samols et al. 1988, J Biol Chem 263, 6461-6464, including homologs in phycobiliprotein producing cyanobacteria, e.g. Gomicki et al. 1993, J Bacteriol 175, 5268-5272; Phung et al., GenBank Accession No. U59235; Nakamura et al. 1998 Nucl Acids Res 26, 63-67. In fact, enzymes sufficient to biotinylate biotinylatable domains have been characterized (e.g. Beckett et al. 1999, Protein Sci 8, 921-929; Buoncristiani et al. 1988, J Biol Chem 263, 1013-1016), permitting in vitro biotinylation (e.g. Li et al., 1992, J Biol Chem 267, 855-863). These biotinylated domains permit especially convenient affinity purification tags (e.g. Cronan 1990, J Biol Chem 265, 10327-10333) and are useful in the many well developed biotin/avidin applications (e.g. Wilchek and Bayer (ed) 1990, Methods Enzymol 184, Academic Press, NY).

In another example, various spacers or flexible linker peptides providing a variety of functionalities, such as a specific endopeptidase recognition and/or cleavage site, an affinity-purification tag, etc., may be used between the heterologous and phycobiliprotein domains. For example, when displayed C-terminally to the phycobiliprotein domain, a specific protease recognition and cleavage site can be engineered immediately upstream from the heterologous protein domain so, upon cleavage with the protease, the heterologous protein domain can be cleanly released from the fusion protein. This strategy also works for most proteins displayed on the N-terminus of the fusion protein because the functions of most heterologous proteins are not affected by C-terminal extensions several residues long. In situations where such C-terminal extension is highly undesirable, an intein domain (Perler F B, Jan. 1, 2000, Nucleic Acids Res 28, 344-345 “InBase, the Intein Database”) can be engineered immediately downstream from the heterologous protein domain. Subsequent excision of intein cleanly releases the displayed domain from the fusion protein.

The linkers may also be used to facilitate display of heterologous protein domains that would otherwise interfere with oligomeric phycobilisome assembly. The length and amino acid sequence requirements of such functionality are readily determined empirically for a given fusion construct. Generally, the linkers are preferably from at least 5, preferably at least 10 residues in length, typically requiring no more than 50, and more often no more than 30 residues. To facilitate an unintrusive orientation, small, flexible residues such as Ala, Gly and Ser are particularly convenient components.

The invention may be practiced in any of a wide variety of cells compatible with expression of the required recombinant fluorescent phycobiliprotein domain, including mammalian cells, yeast cells (e.g. S. cerevisieae), bacterial cells (e.g. E. coli), etc., which may be present in vitro, which is generally isolated from a host, or in situ.

The subject methods include methods for making a functional displayed domain, the method comprising the step of combining a polypeptide comprising a displayed domain and a phycobiliprotein domain with a phycobiliprotein subunit under conditions to form a subject fusion protein. In particular embodiments, the methods further comprise prior to the combining step, the step of making the polypeptide by expressing a nucleic acid encoding the polypeptide; and/or after the combining step, the step of separating the functional displayed domain from the functional phycobiliprotein domain. The methods steps may occur intracellularly, e.g. in a cell which is, or is a progeny of, a natural cell which naturally makes functional phycobiliprotein.

The invention also provides methods of using the subject recombinant cells, including methods for making a fluorescent phycobiliprotein domain by growing a disclosed cell under conditions wherein the cell expresses the fluorescent phycobiliprotein domain, which methods may further comprise the step of isolating the fluorescent phycobiliprotein domain, or the step of detecting location, movement, interactions, appearance, or catabolism of the fluorescent phycobiliprotein domain, heterologous protein domain, or fusion protein thereof, such as within the cell.

In another embodiment, the invention provides phycobiliprotein based transcription reporter cells and assays. For example, the invention includes a recombinant cell which conditionally expresses a heterologous-to-the-cell, fluorescent, first phycobiliprotein domain, which may be expressed independently or as part of a fusion protein as described above. Transcriptional reporter assays are well known in the art. The disclosed phycobiliprotein domain expression systems may be incorporated into any of the many well-known transcriptional reporter assays, used with essentially any promoter compatible with the subject cells, and may be substituted for other transcriptional reporters, such as luciferase and galactosidase, to obtain alternative spectroscopic readouts.

### Fluorescent Analysis Techniques

Proteins of the present invention, including fusion molecules comprising proteins of the invention covalently linked to heterologous molecules, such as carbohydrates, nucleic acids, lipids, and other proteins, can be readily detected both in vitro and in vivo using fluorospectroscopic and fluoromicroscopic techniques common in the art. For example, the fluorescence of cells transformed or transfected with a DNA construct may suitably be measured in a spectrometer where the spectral properties of the cells in liquid culture may be determined as scans of light excitation and emission. Alternatively, such cells grown on nitrocellulose filters placed on plates containing solid media may be illuminated with a scanning polychromatic light source and imaged with an integrating color camera. The color of the emitted light may then be determined by image analysis using specialized software. Exemplary excitation and emission spectra for the phycobiliproteins disclosed herein.

When a fluorophore, such as an phycobiliprotein, is exposed to a light of appropriate wavelength, it will absorb photon energy of given wavelength(s) from the light and later release the stored energy in the form of photons of longer wavelength. The range of wavelengths that a fluorophore is capable of absorbing is the excitation spectrum and the range of wavelengths of light that a fluorophore is capable of emitting is the emission or fluorescence spectrum. The excitation and fluorescence spectra for a given fluorophore usually differ and may be readily measured using known instruments and methods. For example, scintillation counters and photometers (e.g. luminometers), photographic film, and solid-state devices such as charge-coupled devices, may be used to detect and measure the emission of light.

The nucleic acids, vectors, mutant proteins provided herein, in combination with well-known techniques for over-expressing recombinant proteins, make it possible to obtain unlimited supplies of homogeneous phycobiliprotein or their production components. These phycobiliproteins and/or their production components have enhanced and/or different fluorescent properties from those of other currently employed tracers in existing diagnostic and assay systems. Such currently employed tracers include radioactive atoms or molecules, other fluorescent markers, including other phycobiliprotein production components proteins, and color-producing enzymes such as horseradish peroxidase.

The benefits of using phycobiliproteins of the present invention are at least four-fold: phycobiliproteins are safer than radioactive-based assays, phycobiliproteins can be assayed quickly and easily, and large numbers of samples can be handled simultaneously, reducing overall handling and increasing efficiency. Of great significance, the expression and sub-cellular distribution of phycobiliproteins within cells can be detected in living tissues without any other experimental manipulation other than placing the cells on a slide and viewing them through a fluorescence microscope. This represents a significant improvement over, for example, methods of detection that require fixation and subsequent labeling. Even more significantly the phycobiliproteins of the invention may be used un anoxic conditions.

The phycobiliproteins of the present invention can be used in standard assays involving a fluorescent marker. For example, ligand-ligator binding pairs that can be modified with the proteins of the present invention without disrupting the ability of each to bind to the other can form the basis of an assay encompassed by the present invention. These and other assays are known in the art and their use with phycobiliproteins of the present invention will become obvious to one skilled in the art in light of the teachings disclosed herein. Examples of such assays include competitive assays wherein labeled and unlabeled ligands competitively bind to a ligator, noncompetitive assay where a ligand is captured by a ligator and either measured directly or “sandwiched” with a secondary ligator that is labeled. Still other types of assays include immunoassays, single-step homogeneous assays, multiple-step heterogeneous assays, and enzyme assays.

Methods of performing assays on fluorescent materials are well-known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361.

### Immunological Detection of Phycobiliproteins

Phycobiliproteins can be used as an immunogen for the production of antisera or antibodies specific for the protein. The purified protein can be used to screen monoclonal antibodies or antigen-binding fragments prepared by immunization with any of the possible forms of pure and impure preparations containing phycobiliproteins. Recombinant protein is the preferred immunogen for the production of monoclonal or polyclonal antibodies. Alternatively, a synthetic peptide derived from the sequences disclosed herein and conjugated to a carrier protein can be used an immunogen.

Recombinant immunoglobulins may also be produced, see Cabilly, U.S. Pat. No. 4,816,567. These patents are incorporated herein by reference. The polypeptides and antibodies of the present invention may be used with or without modification, including chimeric or humanized antibodies. Immunoassays can be used to qualitatively or quantitatively in the analysis of phycobiliproteins. A general overview of the applicable technology can be found in Harlow & Lane, Antibodies: A Laboratory Manual (1988).

### Antibodies to Phycobiliproteins

Methods of producing polyclonal and monoclonal antibodies that react specifically with phycobiliproteins, or phycobiliprotein fragments, are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature, 256:495-497 (1975). Such techniques include antibody preparation by selection of antibodies from libraries of recombinant antibodies in phage or similar vectors, as well as preparation of polyclonal and monoclonal antibodies by immunizing mammals (see, e.g., Huse et al., Science, 246:1275-1281 (1989); Ward et al., Nature, 341:544-546 (1989)).

Antibodies can be raised against folded or denatured phycobiliproteins, the difference being that antibodies to folded phycobiliproteins are more likely to recognize epitopes which are only present in the folded protein. Antibodies, including binding fragments and single chain versions, against predetermined fragments of phycobiliproteins can also be raised by immunization of animals with conjugates of the fragments with immunogenic proteins by methods known in the art.

### Monoclonal Antibodies

Monoclonal antibodies can be prepared from various mammalian hosts, such as rodents, cows, sheep, goats, donkeys, primates, humans, etc. Description of techniques for preparing such monoclonal antibodies may be found in, e.g., Stites, et al. (eds.) Basic and Clinical Immunology (4.th ed.), Lange Medical Publications, Los Altos, Calif., and references cited therein; Harlow and Lane (1988) Antibodies: A Laboratory Manual, CSH Press; Goding (1986) Monoclonal Antibodies: Principles and Practice (2nd ed.) Academic Press, New York; and particularly in Kohler & Milstein, Eur. J. Immunol., 6:511-519 (1976).

Techniques involving in vitro exposure of lymphocytes to the antigenic polypeptides or alternatively to selection of libraries of antibodies in phage or similar vectors are described in Huse, et al., Science, 246:1275-1281 (1989); and Ward et al., Nature, 341:544-546 (1989), each of which is hereby incorporated herein by reference.

Monoclonal antibodies and polyclonal sera are tested for specificity by titering against the immunogen protein in an immunoassay, for example, a solid phase immunoassay with the immunogen immobilized on a solid support. Typically, polyclonal antisera with a titer of 10⁴ or greater are selected and tested for their cross reactivity against non-phycobiliproteins using a competitive binding immunoassay. Specific polyclonal antisera and monoclonal antibodies will usually bind with a Kₐ of at least about 1 mM, more usually at least about 300 μM, preferably at least about 3 μM or better, and most preferably, 0.03 μM or better. Antibodies specific only for a particular phycobiliprotein orthologs, can also be made, by subtracting out other cross-reacting orthologs from another species. Exemplary binding assays are described in more detail below.

Antibody titers of ascites or hybridoma culture fluids are determined by various serological or immunological assays which include, but are not limited to, precipitation, passive agglutination, enzyme-linked immunosorbent assay (ELISA) technique and radioimmunoassay (RIA) techniques. Similar assays are used to detect the presence of phycobiliproteins in culture media or tissue and cell extracts.

### Polyclonal Antibodies

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the protein. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Once the specific antibodies against a phycobiliproteins are available, the phycobiliproteins can be detected by a variety of immunoassay methods. For a review of immunological and immunoassay procedures, see Basic and Clinical Immunology (Stites & Terr eds., 7th ed., 1991). Moreover, the immunoassays of the present invention can be performed in any of several configurations, which are reviewed extensively in Enzyme Immunoassay (Maggio, ed., 1980); and Harlow & Lane, supra.

### Immunological Binding Assays

Phycobiliproteins of the invention can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case phycobiliprotein or an antigenic subsequence thereof). The antibody (e.g., anti-phycobiliprotein) may be produced by any of a number of means well-known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled phycobiliprotein polypeptide or a labeled anti-phycobiliprotein antibody. Alternatively, the labeling agent may be a third moiety, such a secondary antibody, which specifically binds to the antibody/phycobiliprotein complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol., 111:1401-1406 (1973); Akerstrom et al., J. Immunol., 135:2589-2542 (1985)). The labeling agent can be modified with a detectable moiety, such as biotin, to which another molecule can specifically bind, such as streptavidin. A variety of detectable moieties are well-known to those skilled in the art.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

### Non-Competitive Assay Formats

Immunoassays for detecting phycobiliproteins in samples may be either competitive or non-competitive. Non-competitive immunoassays are assays in which the amount of antigen is directly measured. In one preferred “sandwich” assay, for example, the anti-phycobiliprotein subunit antibodies can be bound directly to a solid substrate on which they are immobilized. These immobilized antibodies then capture phycobiliprotein present in the test sample. phycobiliproteins are thus immobilized and then bound by a labeling agent, such as a second phycobiliprotein antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

### Competitive Assay Formats

In competitive assays, the amount of phycobiliprotein present in the sample is measured indirectly by measuring the amount of known, added (exogenous) phycobiliprotein displaced (competed away) from an anti-phycobiliprotein antibody by the unknown phycobiliprotein present in a sample. In one competitive assay, a known amount of phycobiliprotein is added to a sample and the sample is then contacted with an antibody that specifically binds to phycobiliprotein. The amount of exogenous phycobiliprotein bound to the antibody is inversely proportional to the concentration of the phycobiliprotein present in the sample. In a particularly preferred embodiment, the antibody is immobilized on a solid substrate. The amount of phycobiliprotein bound to the antibody may be determined either by measuring the amount of phycobiliprotein present in an phycobiliprotein/antibody complex, or alternatively by measuring the amount of remaining uncomplexed protein. The amount of phycobiliprotein may be detected by providing a labeled phycobiliprotein molecule.

A hapten inhibition assay is another preferred competitive assay. In this assay the known phycobiliprotein is immobilized on a solid substrate. A known amount of anti-phycobiliprotein antibody is added to the sample, and the sample is then contacted with the immobilized phycobiliprotein. The amount of anti-phycobiliprotein antibody bound to the known immobilized phycobiliprotein is inversely proportional to the amount of phycobiliprotein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

### Cross-Reactivity Determinations

Immunoassays in the competitive binding format can also be used for cross-reactivity determinations for phycobiliprotein. For example, a phycobiliprotein protein corresponding to those herein or an immunogenic region thereof can be immobilized to a solid support. Other proteins, such as other phycobiliproteins, are added to the assay so as to compete for binding of the antisera to the immobilized antigen. The ability of the added proteins to compete for binding of the antisera to the immobilized protein is compared to the ability of phycobiliproteins or immunogenic portion thereof to compete with itself. The percent cross-reactivity for the above proteins is calculated, using standard calculations. Those antisera with less than 10% cross-reactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele, ortholog, or polymorphic variant of phycobiliprotein, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein encoded by phycobiliprotein that is required to inhibit 50% of binding, then the second protein is said to specifically bind to the polyclonal antibodies generated to the respective phycobiliprotein immunogen. Therefore, by definition, a protein that specifically binds to antibodies generated to a phycobiliprotein is an allele, ortholog, or polymorphic variant of phycobiliprotein and a member of the genus of phycobiliproteins comprising the present invention.

### Other Assay Formats

Western blot (immunoblot) analysis is used to detect and quantify the presence of phycobiliprotein in the sample. The technique generally comprises separating sample proteins by gel electrophoresis on the basis of molecular weight, transferring the separated proteins to a suitable solid support, (such as a PVDF membrane, a nitrocellulose filter, a nylon filter, or derivatized nylon filter), and incubating the sample with the antibodies that specifically bind phycobiliprotein. The anti-phycobiliprotein antibodies specifically bind to phycobiliprotein on the solid support. These antibodies may be directly labeled or alternatively may be subsequently detected using labeled antibodies (e.g., labeled sheep anti-mouse antibodies) that specifically bind to the anti-phycobiliprotein antibodies.

### Reduction of Non-Specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well-known to those of skill in the art. Typically, this technique involves coating the substrate with a proteinaceous composition. In particular, protein compositions such as bovine serum albumin (BSA), non-fat powdered milk, and gelatin are widely used with powdered milk being most preferred.

### Fusion Constructs

A particularly useful application of the phycobiliproteins of the present invention is their use as molecular markers or “tags”. By covalently linking or “fusing” to other molecules, including but not limited to lipids, nucleic acids, polysaccharides, proteins and synthetic polymers, one can easily track the location (and often the concentration) of the tagged molecule using fluorometric techniques previously described. Properly tagged molecules can often be tracked in living organisms, adding a dynamic to the study of cellular processes unattainable prior to the discovery of phycobiliproteins. Moreover, the present invention encompasses the fusion of PEST sequences to phycobiliprotein, thereby decreasing the half-life of the phycobiliprotein and any molecule tagged with the phycobiliprotein PEST marker. Modification of the half-life of a phycobiliprotein tag facilitates the study of dynamic cellular processes by preventing the build up of tagged fluorescent end-products of the process being studied.

The DNA sequence encoding the fluorescent protein of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol., 132:3459-3465 (1986); U.S. Pat. No. 4,882,279).

Linking agents can be either zero length (directly fuses two molecules together without the introduction of extrinsic material) or can create “spacers” of variable lengths that allow greater separation between the fused molecules. In designing a linking agent, it is important to consider both desired internal and external characteristics. Internally, it is important to choose molecules and binding groups that work well together, ones that do not interact with each other in order to lessen desired functionality. In addition, it is important to maintain an appropriate distance between the fused components so that they are able to operate simultaneously. For example, a carbon chain linker could be incorporated between the two regions (such as (N-substituted) maleimide-(CH₂)₆-His₆), in order to allow a His-tag to be sufficiently far from the target molecule such that binding of the linking agent to a target molecule does not sterically interfere with the ability of the tag to bind to a nickel column. Desirable external characteristics include selecting a binding group that will bind with a desired specificity to the target macromolecule. In some cases, it might be desirable to create a linking agent that will bind reversibly to a target protein. For example, the use of a linking agent that forms reversible fusions, such as disulfide bonds between cysteines that could be reversed by a reducing agent such as DTT. Such linking agents include those capable of forming disulfide (—S—S—), glycol (—CH(OH)—CH(OH)—), azo (—N═N—), sulfone (—S(═O₂)—), or ester (—C(═O)—O—) bridges.

Binding groups reactive with proteins and other polymers are well-known in the literature concerned with creating bifunctional molecules that will act as cross-linking agents. For example, Wong provides a wealth of information on designing and selecting appropriate molecules (see Wong, Chemistry of Protein Conjugation and Cross-Linking, CRC Press, 1991 and references therein). An appropriate choice is typically a binding group that reacts relatively non-specifically with the target molecule, but does not react with any component of itself (a binding group that reacts with histidine side chains, for example, would not typically be considered a good choice when using a His-tag, though such a reaction could produce very strong signals akin to the self-reactions in the ubiquitination pathway). Commonly used agents suitable for use as binding groups include aryl halides (which react with histidine side chains), N-maleimide derivatives (react with —SH and —NH₂), mercurials (react with —SH), disulfides (react with —SH), acid anhydrides (react with —NH₂ and phenols), isocyanates (react with —NH₂), isothiocyanates (react with —NH₂), sulfonyl halides (react with —NH₂), imidoesters (react with —NH₂), diazoacetates (react with —COOH and —SH) and dicarbonyl compounds (react with —NH—C(NH)—NH₂). Appropriate reactions conditions for using these binding groups are well-known to those of ordinary skill in the art.

Linking agents can also be homo- or heterobifunctional, depending upon whether the reactive binding groups are the same or different. Among the homo-bifunctional coupling agents, there may be mentioned DITC (1,4-phenylene diisothiocyanate), DSS (disuccinimidyl suberate) or the like. Among the hetero-bifunctional coupling agents are SMCC (succinimidyl-4-(N-maleimidomethyl)cyclohexane-1-carboxylate), or SMPB (succinimidyl-4-(p-maleimidophenyl)butyrate) which are capable of reacting, on the one hand, with a primary amine and, on the other hand, with a thiol. A vast majority of the hetero-bifunctional cross-linking agents contains a primary amine-reactive group and a thiol-reactive group. A novel hetero-bifunctional linker for formyl to thiol coupling was disclosed by Heindel et al., Bioconjugate Chem., 2:427-430 (1991).

After coupling the ligand with the copolymer, the possible excess reactive functional groups of the copolymer are neutralized by methods known in the art. For example, the aldehyde groups in excess can be neutralized with a primary amine such as ethanolamine, the maleimide or haloalkyl groups can be neutralized with a thiol (such as thioethanolamine or dithiothreitol), and so on.

Linking agents particularly useful for fusing proteins are those that induce condensation of carboxy and primary amino groups to form an amide bond, such as carbodiimides, ethylchloroformate, Woodward's reagent K1, carbonyldiimidazole, etc.

Polysaccharides can be made reactive through chemical or enzymatic oxidation of oligosaccharides to form aldehyde-binding groups. Such binding groups can react with compounds containing, for example, amines hydrazines, hydrazides, or semicarbazides. Carbohydrate-directed hetero-bifunctional cross-linking agents are, for example, disclosed U.S. Pat. No. 5,329,029.

If the nucleic acid sequence encoding a protein has been isolated, then the protein can be tagged indirectly through recombinant genetic techniques. For example, a PEST sequence can be ligated in frame to the 5′ end of the coding sequence of a phycobiliprotein. Subsequent expression of the fused nucleotide, by methods described above, results in the production of a recombinant phycobiliproteins-PEST protein with a shortened half-life when expressed intracellularly compared to that of the wild-type phycobiliprotein. The process can be taken a step further by fusing in frame with the phycobiliprotein coding sequence, a nucleic acid encoding a target protein. Subsequent expression of this heterologous construct results in a fluorescently-tagged target protein, where the tag (phycobiliprotein) and possibly the entire recombinant protein has a shortened half-life. Using recombinant DNA technology, the proteins may be fused with no intervening extrinsic linker peptide, or the proteins may be fused through an intervening “spacer” peptide of any length. Spacers are incorporated simply by inserting a nucleic acid encoding the desired spacer between, and in frame with, the nucleic acids encoding the two proteins to be fused. Production and expression of such DNA constructs may be performed using techniques common in the art and discussed above.

Recombinant DNA techniques are the method of choice for introducing PEST sequences into phycobiliproteins. PEST sequences are polypeptide sequences enriched in Proline (P), glutamic acid (E), serine (S) and threonine (T), and target proteins for rapid destruction. Scientific research has provided strong evidence that PEST sequences do, in fact, serve as proteolytic signals. See for example, Rogers et al., Science 234:364-368 (1986); Rechsteiner et al., TIBS, 21:267-271 (1996).

### Diagnostic and Therapeutic Uses

The engineered fluorescent proteins of this invention are useful as fluorescent markers in the many ways fluorescent markers already are used. This includes, for example, coupling phycobiliprotein to antibodies, nucleic acids or other receptors for use in detection assays, such as immunoassays or hybridization assays. Such constructs are particularly useful in applications involving the monitoring of gene expression and protein localization. Phycobiliproteins are ideal for such applications as they are readily detectable, can be detected on irradiation using standard long-wave UV light sources; offers the possibility of real-time detection in vivo; the introduction of a substrate is not required to produce a signal; and its relatively small size (25.2 kDa) and monomeric nature, making protein fusions manageable.

### Cell Dynamics

Phycobiliprotein of this invention are useful to track the movement of proteins in cells. For example, a nucleic acid encoding phycobiliprotein is fused to a nucleic acid molecule encoding the protein of interest in an expression vector. Upon expression inside the cell, the protein of interest can be localized based on fluorescence.

Phycobiliproteins may be used to tag virtually any protein and to follow the location of the protein under different conditions. For example, in following a given protein through meiosis, mitosis, apoptosis or other cellular processes. The location of a given protein can also be determined in response to a number of external stimuli. Such stimuli include different physical conditions, such as increasing or decreasing temperature, and also different chemical environments. By the term “chemical environment”, it is meant both natural environments that may be encountered, such as compositions with different levels of salt or serum growth factors and the like, and also compositions to which an effector molecule has been added.

For the study of protein localization, concatenation of phycobiliprotein and a gene of interest encoding for a cellular protein, and subsequent expression of the resulting fusion protein, results in a fluorescent fusion protein that is localized at the normal intracellular location of the protein encoded by the gene of interest. Identifying the intracellular location of phycobiliprotein thus identifies the intracellular location of the protein of interest. The use of such fusion proteins yields information on the normal cellular role of the protein encoded by the gene of interest.

Florescence Resonance Energy Transfer (FRET)

Fluorescent molecules are useful in fluorescence resonance energy transfer (“FRET”). FRET involves a donor molecule and an acceptor molecule. To optimize the efficiency and detectability of FRET between a donor and acceptor molecule, several factors need to be balanced. The emission spectrum of the donor should overlap as much as possible with the excitation spectrum of the acceptor to maximize the overlap integral. Also, the quantum yield of the donor moiety and the extinction coefficient of the acceptor should likewise be as high as possible to maximize R_{O}, the distance at which energy transfer efficiency is 50%. However, the excitation spectra of the donor and acceptor should overlap as little as possible so that a wavelength region can be found at which the donor can be excited efficiently without directly exciting the acceptor. Similarly, the emission spectra of the donor and acceptor should overlap as little as possible so that the two emissions can be clearly distinguished. Preferably, the efficiency of FRET between the donor and acceptor is at least 10%, more preferably at least 50% and even more preferably at least 80%.

Engineered phycobiliproteins of the present invention enhance FRET by extending the repertoire of donor and acceptor fluorophores, allowing greater selection of fluorophore pairs in designing FRET studies. Moreover, greater selection of FRET donor/acceptor pairs offers the possibility of tracking multiple events, each associated with a donor/acceptor pair having distinct spectral characteristics from other pairs in the assay.

Uses of phycobiliproteins in FRET analysis include detecting the cleavage of a substrate having the donor and acceptor coupled to the substrate on opposite sides of the cleavage site. Upon cleavage of the substrate, the donor/acceptor pair are physically separated, eliminating FRET. Assays involve contacting the substrate with a sample, and determining a qualitative or quantitative change in FRET.

Another example is the use of FRET to detect changes in potential across a membrane. A donor and acceptor are placed on opposite sides of a membrane such that one translates across the membrane in response to a voltage change. This creates a measurable FRET.

### Cell Labeling

It will be appreciated by one of ordinary skill in the art that cells that have been transfected with exogenous DNA can also be identified without creating a fusion protein. The method relies on the identification of cells that have received and express a plasmid or vector that comprises at least a nucleic acid encoding phycobiliprotein. Cells can be transfected with such a vector by any of the methods known in the art, as detailed above. In the case of stable transformations, both the initially transformed cell and its progeny will carry and express the gene for phycobiliprotein, consequently fluorescing when exposed to ultra violet light. The technique is particularly useful in cancer and embryological studies. For example, progenitor cells in early embryos can be stably transformed with a vector comprising a nucleic acid encoding phycobiliprotein. The progenitor cell and its progeny can then be followed throughout the course of development simply by exposing the cells to ultra violet light. Likewise, metastatic cells can be similarly labeled and tracked. Such tracking would allow scientists to follow, for example, the efficacy of treatment regimes in model systems, as the labeled cells not only provide a means for determining the rate of tumor growth, but also the presence and extent of metastatic growths in a simple and quantitative manner.

### Fluorescent Activated Cell Sorting (FACS)

Many conventional FACS methods require the use of fluorescent dyes conjugated to purified antibodies. Proteins tagged with a fluorescent label would be preferred over antibodies in FACS applications because cells do not have to be incubated with the fluorescent-tagged reagent and because there is no background due to nonspecific binding of an antibody conjugate. Phycobiliproteins are particularly suitable for use in FACS as fluorescence is stable and species-independent and does not require any substrates or cofactors. Moreover, genes of therapeutic interest often do not produce an easily distinguishable phenotype in cells expressing such a gene. Consequently, such a therapeutic gene is usually inserted into a vector that contains a marker gene. The therapeutic gene and the marker gene are placed in the vector under the control of a cellular or viral promoter, and introduced into mammalian cells of interest; subsequently, the transfected cells (the cells containing the vector) are selected according to the phenotype determined by the marker gene. The use of phycobiliprotein for selection obviates the need to grow the mammalian cells of interest in the presence of drugs in order to select for the transfected cells. Cells transfected with a vector containing phycobiliprotein and the gene(s) of therapeutic interest are recognized by their fluorescence following excitation, and are sorted by FACS.

In another application, phycobiliprotein is used to select specific cell lines in which expression vectors that have integrated at a chromosomal location giving very high expression of phycobiliprotein and of a second gene. A phycobiliprotein expression vector is transfected into mammalian cells along with a vector expressing a gene of interest. The two vectors become integrated into the chromosome together, and selection of brightly fluorescing cells will yield the cells with high levels of expression of the gene of interest.

### Inducible Promoters

The engineered phycobiliproteins of this invention are useful in systems to detect induction of transcription. In certain embodiments, a nucleotide sequence encoding the engineered phycobiliprotein is fused to expression control sequences of interest and the expression vector is transfected into a cell. Induction of the promoter can be measured by detecting the expression and/or quantity of fluorescence. Such constructs can be used to follow signaling pathways from receptor to promoter.

Phycobiliprotein can also be used to select bacterial promoters that are induced in response to a specific stimulus. Such an application allows for the systematic scanning of chromosomes of pathogenic and/or commercially important organisms for genes that are regulated in response to environmental stimuli such as iron starvation, transient stress, and antimicrobial agents.

Nucleic acids encoding phycobiliproteins also provide another dimension to the analysis of promoters in mammalian cells. A range of promoters can be tested for their suitability for use with a given gene, cell, or system. This applies to in vitro uses, such as in identifying a suitable promoter for use in recombinant expression and high-level protein production, and also in in vivo uses, such as in pre-clinical testing or in gene therapy in human subjects.

The testing and ultimate use of inducible and tissue-specific promoters forms another aspect of this invention. In recombinant expression for the purposes of protein production, it may be desired to induce expression at a particular stage of the cell culture or cell cycle, or in a particular cell type. In analyzing the distribution of a given protein within a cell or a given system, it is useful to use a promoter that is only switched on under certain conditions, such as in the presence of certain cytokines or hormones.

In screening embodiments, nucleic acids encoding phycobiliprotein will be positioned downstream of a promoter that is known to be inducible by the agent that one wishes to identify. Expression of phycobiliprotein in the cells will normally be silent, and will be switched on by exposing the cell to a composition that contains the selected agent. In using a promoter that is responsive to, for example, a heavy metal, a toxin, a hormone, a cytokine or other defined molecule, the presence of a heavy metal, toxin, hormone, cytokine or such like can readily be determined.

Standard biological applications of phycobiliprotein should not be overlooked. For example, its use as a molecular weight marker on protein gels and Western blots, in calibration of fluorometers and FACS machines and in microinjection into cells and tissues.

### Kits

Expression kits comprising nucleic acids encoding phycobiliprotein production components, or the phycobiliproteins themselves form another aspect of the invention. Such kits will generally contain, in suitable container means, a formulation of a nucleic acid encoding a phycobiliprotein production components or a vector capable of expressing such a nucleic acid, and instructions for using the same.

When the components of the kit are provided in one or more liquid solutions, the liquid solution is an aqueous solution, with a sterile aqueous solution being particularly preferred. The phycobiliprotein production components genes or vectors may also be formulated into a syringeable composition. If such is the case, the container means may itself be a syringe, pipette, eye dropper, or other such like apparatus, from which the formulation may be applied to a cell, or to an area of the body, or injected into an animal, or applied to and mixed with other components of a kit.

Components of the kit may be provided as dried powder(s). When reagents or components are provided as a dry powder, the powder can be reconstituted by the addition of a suitable solvent. It is envisioned that the solvent may also be provided in another container means.

The container means will generally include at least one vial, test tube, flask, bottle, syringe or other container means, into which the phycobiliprotein production components gene or vector may be suitably allocated. A second engineered nucleic acid encoding phycobiliprotein production components or vector construct may also be provided, wherein the kit will also generally contain a second vial or other container into which this is placed. The kits may also comprise a second/third container means for containing a sterile, pharmaceutically acceptable buffer or other diluent.

Kits of the present invention will also typically include a means for containing the vials in close confinement for commercial sale, such as, e.g., injection or blow-molded plastic containers into which the desired vials are retained.

Irrespective of the number or type of containers, the kits of the invention may also comprise, or be packaged with, one or more further molecular biological reagents, such as restriction enzymes.

### EXAMPLES

### Example 1

### Attachment of Non-Cognate Chromophores to CpcA of Synechocystis sp. Strain PCC 6803 by Heterologous Expression in Escherichia coli

Cyanobacteria have developed a number of pigmented proteins to collect light energy optimally for photosynthesis. Most utilize finely tuned antennae known as phycobilisomes, which are supramolecular structures composed of both chromophorylated and non-chromophorylated proteins. The chromophorylated components, i.e., the phycobiliproteins, carry covalently bound, linear tetrapyrroles (phycobilins) that are responsible for the light-harvesting properties of these proteins. Only four bilins are known to be incorporated into cyanobacterial phycobiliproteins: phycocyanobilin (PCB) and phycoerythrobilin (PEB) and their respective Δ5-to-Δ2 double-bond isomers, phycoviolobilin (PVB) and phycourobilin (PUB) (see Table 1). In addition to its role in light harvesting, PCB also has a role in light sensing, as it has been found to be the chromophore attached to many cyanobacterial phytochromes. Unlike cyanobacteria, plants use the bilin phytochromobilin (PΦB) only for light sensing and do not use bilins for light harvesting. PΦB and PCB differ only in the presence of an 18-vinyl group on PΦB (Table 1). This change in the conjugation state of the molecule shifts its absorption maximum to the red by approximately 10 nm. Photoreceptors utilizing PCB, PΦB, or PVB are thought to be capable of responding to the wavelengths of light in their environments due to a light-induced isomerization reaction around the 15-16 double bond of the molecules. Although the biological significance of the reaction remains unclear, this light-induced isomerization reaction also occurs within the phycoerythrocyanin α subunit (PecA), a phycobiliprotein that carries the PVB chromophore. Because this double bond is not present in PEB or PUB, these bilins are unable to serve as chromophores for the phytochrome-type of light sensing. It should be noted that many bacteria also contain phytochrome-like photoreceptors, although the chromophore in these proteins is biliverdin.

The biosynthetic pathways for the production of the four cyanobacterial phycobilins and phytochromobilin in the Arabidopsis thaliana have been elucidated. These five naturally occurring, linear tetrapyrrole chromophores share a common precursor, biliverdin IXα, which is the product of heme ring linearization by heme oxygenase. Biliverdin IXα is subsequently reduced by one of several possible bilin reductases. Plants utilize only HY2, which reduces the vinyl pyrrole A ring of biliverdin IXα to form the ethylidene moiety, phytochromobilin (PΦB). Cyanobacteria utilize phycocyanobilin:ferredoxin oxidoreductase (PcyA) to perform a two-step reaction; the reduction of the vinyl pyrrole A ring of biliverdin IXα as is done by HY2 and the reduction of the 18-vinyl group to yield PCB. PEB can be produced by either of two pathways. The PebA-PebB pathway is found in many cyanobacteria, which use PebA to reduce biliverdin IXα to 15, 16 dihydrobiliverdin (DHBV), and then use PebB to reduce 15, 16 DHBV further to PEB. Alternatively, PebS from the myovirus P-SSM4 can perform both reactions in a manner similar to the two-step reduction of biliverdin to PCB by PcyA. PVB and PUB do not occur as free bilins in cyanobacteria cells; instead, these two bilins are formed by isomerizing lyases that convert PCB and PEB to PVB and PUB, respectively, and attach them to cysteines of the appropriate chromoproteins.

Along with the reductases that function to synthesize the phycobilins, proteins known as the phycobiliprotein lyases have also been the subject of intensive study in recent years. These enzymes attach chromophores to appropriate apoproteins, and in some instances can exchange chromophores between holo- and apo-phycobiliprotein subunits. Although several chromoproteins, including plant phytochromes, some cyanobacterial phytochromes, bacterial phytochromes, and the ApcE subunit of the phycobilisome core, have been found to have auto-lyase activity, all other phycobiliproteins require one or more lyases for chromophore attachment to produce the holo-protein. CpcE/CpcF, the first identified phycobiliprotein lyase, is a heterodimer responsible for attachment of PCB to CpcA, and this enzyme exhibits no lyase activity with any other phycobiliprotein subunit. The related lyase, PecE/PecF, catalyzes the formation of PVB from PCB and attaches this chromophore to PecA. Other characterized lyases include the CpcS or the CpcS/CpcU heterodimer lyases, both of which can attach PCB to conserved Cys residues at the 82/84 positions of CpcB, ApcA, ApcB, ApcD, and ApcF; and the CpcT lyase, which attaches PCB to Cys153 residue of CpcB. The discovery of the CpcS/CpcU and CpcT lyase family in Synechococcus sp. strain PCC 7002 has allowed the lyase requirements for all phycobiliproteins synthesized in this organism to be determined. Recently RpcG, which appears to be a fusion of PecE- and PecF-type domains, has been reported to attach both PVB and PUB to RpcA, its cognate apoprotein. Lyases which are homologous to the CpcS/CpcU/CpcT family and are likely to be responsible for the attachment of PEB in phycoerythrins, have been identified in the genomes of numerous species. These enzymes are largely uncharacterized but are likely to function similarly. This promises to be an interesting area of research, because phycoerythrins have more chromophores than phycocyanins, and thus they are likely to require additional lyases for their post-translational maturation.

In cyanobacteria that synthesize both PEB and PCB, energy transfer could be severely negatively impacted if PEB were inappropriately attached to ApcE, one of the terminal energy emitters from the PBS. Similarly negative impacts on energy transfer might arise from inappropriate attachment of PEB to other allophycocyanin subunits or to Cys82 of CpcB. Because inappropriate phycobilin attachment is not observed, it has generally been assumed that PBP lyases must exhibit a very high degree of substrate specificity in vivo. Mis-incorporation of chromophores into biliproteins has been achieved artificially, however. For example, PEB can be auto-catalytically bound by phytochrome to form a highly fluorescent protein which was termed a phytofluor, and plants lacking HY2 can be complemented with pcyA, which leads to PCB incorporation into the phytochromes. Similarly, complementation of a pcyA mutant with HY2 in Synechococcus sp. PCC 7002 leads to the complete replacement of all PCB chromophores by PΦB (R. M. Alvey and D. A. Bryant, manuscript in preparation). Mutants of Nostoc sp. PCC 7120 lacking the PecE/PecF lyase have been found to produce small amounts of PecA carrying PCB as well.

The complexity of phycobiliprotein biogenesis, in combination with a poor understanding of the enzymes responsible for these post-translational modifications, has been a hindrance to widespread adoption of phycobiliproteins as markers and probes. However, recent advances in our understanding of phycobiliprotein biogenesis have begun to eliminate this barrier. Experiments conducted using Escherichia coli and the CpcA/CpcE/CpcF and PecA/PecE/PecF systems have allowed the production of holo-phycobiliproteins in this heterologous host. Tooley et al. described a two plasmid system that led to the production of phycobiliproteins in E. coli. In this system, one plasmid encoded heme oxygenase (hox1) and phycocyanobilin oxidoreductase (pcyA) to produce the PCB chromophore. The other plasmid carried the CpcA, CpcE and CpcF genes, which encode the phycocyanin α subunit and its corresponding heterodimeric lyase (CpcE-CpcF). Heterologous production of the α subunit of phycoerythrocyanin was subsequently reported using a similar dual plasmid system but with pecA, pecE and pecF. In the studies reported here, we have significantly extended these initial observations to probe the substrate specificity of bilin lyases in order to understand lyase function better and to create unique, highly fluorescent phycobiliproteins, some of which have novel properties that do not occur naturally.

### Experimental Procedures (Materials and Methods)

Plasmid Construction

Several plasmids used in the studies reported here had been used in previous studies, and their construction is described elsewhere (see Table 1 for references). The HY2 gene was amplified from the plasmid pPL-PΦB (29) using the primers ECHY2F (SEQ ID NO:1): cagaattcgtctctgctgtgtcgtataaggaGttcgcagag (an A to G silent mutation (capital “G” in the primer sequence) was introduced to eliminate an EcoRI site and a separate EcoRI site (in bold) was added near the 5′ end of the primer) and ECHY2R (SEQ ID NO:2): aagtcgacgatttagccgataaattgtcctgtta (SalI site in bold). The corresponding HY2 amplicon was cloned into pPcyA by removing pcyA (using EcoRI and SalI) and inserting appropriately digested HY2 PCR to yield plasmid pHY2. Plasmids containing pecE and/or pecF were made by PCR amplification of pecE (using primers pecEF (SEQ ID NO:3): attgacgtcgacaaggagcttgcatgtgactgctgaaccaattcttt and pecER (SEQ ID NO:4): gtgcggccgcgacgcttttaaagttgaattaataaatcatcaat) which contain SalI and NotI sites (in bold) and pecF (SEQ ID NO:5) (using primers pecFF: attagcggccgctaggagggctaacatatgaatcaagcttcattgagcg and pecFR (SEQ ID NO:6): gagggacctagaagactaactcaaggcgatcgcca) which contain EagI and BamHI sites (in bold) from the genome of Nostoc sp. strain PCC 7120 (26) (GenBank accession no. AF178757). These DNA fragments along with CpcE, CpcF (both from pBS414v) were cloned into pBS405v to produce plasmids pBS405vpecEF, pBS405vpecE, pBS405vpecF, pBS405vCpcEpecF, pBS405vpecEcpcF, pBS405vCpcE, and pBS405vcpcF (see Table 1).

### In Vivo Heterologous Expression and Purification of Recombinant Proteins

Plasmids for the expression of various combinations of bilins, lyases, and apo-phycobiliproteins were transformed in to E. coli BL21 (DE3) cells. Transformants were grown on media containing both chloramphenicol (34 μg ml⁻¹) and spectinomycin (50 μg ml⁻¹) to ensure the presence of the plasmid leading to chromophore production and apoprotein/lyase, respectively. For expression studies, a 5-ml overnight starter culture was added to 1.0 l of LB medium containing antibiotics, and the cultures were shaken at 150 rpm at 37° C. for 3-4 h until the optical density at 600 nm reached 0.6, at which time gene expression was induced by the addition of isopropyl β-D thioglalactoside (final concentration 1 mM). Cultures were incubated with shaking at 18° C. overnight and were harvested by centrifugation at 10,000×g for ten minutes and washed once in Buffer O (50 mM Tris-HCl, 150 mM NaCl, pH 8.0). Cell pellets were stored at −20° C. until required. Cells containing recombinant proteins were thawed, resuspended in Buffer O (20 ml of buffer was used for the cells harvested from 1.0 l of culture), and lysed by three passages through a chilled French pressure cell at 138 MPa. The lysed cell suspension was centrifuged for 35 min at 35,000×g to remove unbroken cells and large cell debris. [His₁₀]-tagged recombinant proteins were purified by affinity chromatography on columns (1.0-ml bed volume) containing Ni-NTA H is bind resin (Novagen-EMD, La Jolla, Calif.), and the protein was eluted by Buffer O containing 250 mM imidazole (30). Recombinant proteins were dialyzed with buffer O overnight at 4° C. to remove the imidazole. Purified proteins were stored at −20° C. until analyzed.

### Absorbance and Fluorescence Measurements

Absorbance spectra were acquired using a Genesys 10 spectrophotometer (Thermo Fisher Scientific, Waltham, Mass.). Fluorescence emission spectra were recorded using an SLM 8000C spectrofluorometer modernized for computerized data acquisition by On-Line Systems, Inc. (Bogart, Ga.). The excitation monochromator was set to 570 nm for PCB- and PΦB-CpcA and to 530 nm for PEB-, PVB-, and PtVB-CpcA. Fluorescence quantum yield measurements were made using the comparative methods described by Nanoco Technologies (www.nanocotechnologies.com/download.aspx?ID=77) and Jobin Yvon Ltd. (www.jobinyvon.com/usadivisions/Fluorescence/applications/quantumyieldstrad.pdf). The standard used for this measurements is cresyl violet perchlorate (Sigma Chemical Co., St. Louis, Mo.), which has a quantum yield of 0.59 in ethanol.

### Polyacrylamide Gel Electrophoresis and Fluorescence Imaging

Proteins were analyzed by polyacrylamide gel electrophoresis in the presence of SDS as previously described. Gels containing chromophorylated proteins were treated with a solution of 25 mM ZnCl₂, to enhance the fluorescence from the bilins, and gels were scanned with a Typhoon 8600 Variable Mode Imager (GE Healthcare Lifesciences, Pittsburgh, Pa.) using an excitation wavelength of 532 nm. After fluorescence imaging analysis, gels were stained with Coomassie Brilliant Blue R-250 for protein detection.

### Photoreversibility

To determine if samples exhibited photochemistry, absorbance spectra were taken immediately before subjecting as-isolated samples to continuous illumination at their peak absorbance maxima for up to 2 h. Illumination was provided from 400 W tungsten-halogen lamp (model 66057, Oriel Corp., Stratford, Conn.) using a ¼ meter monochromator (Model 82-410, Jarrell-Ash Co., Waltham, Mass.). After illumination, the absorption spectra of the samples were immediately measured again to determine whether any light-induced changes in absorption had occurred. Samples exhibiting changes in absorption were subsequently treated with monochromatic light at the newly formed absorption maximum to determine if the change was photoreversible.

### Results

Heterologous Production of Holo-HT-CpcA-PCB in E. coli by Coexpression of hox1, pcyA, CpcE, CpcF, and HT-CpcA

When HT-CpcA was co-expressed in E. coli with the hot, pcyA, CpcE, and CpcF genes, encoding heme oxygenase 1, phycocyanobilin oxidoreductase, and the phycocyanin α subunit phycocyanobilin lyase, respectively, the IPTG-induced E. coli cells were a bright blue color after overnight incubation at 18° C. Purified HT-CpcA-PCB had an absorption maximum at 625 nm, a fluorescence emission maximum at 646 nm, and a fluorescence quantum yield of 0.39. The absorption and fluorescence emission properties of the protein are similar to those reported for other phycocyanin α subunits. These results showed that HO1 and PcyA efficiently produced PCB from endogenously synthesized heme and that the CpcE/CpcF lyase effectively attached the PCB synthesized to apo-HT-CpcA, forming HT-CpcA-PCB (i.e., holo-HT-CpcA).

### Production of HT-CpcA-PEB Using the Alternative Biliverdin Reductase, Phycoerythrobilin Synthase (PebS)

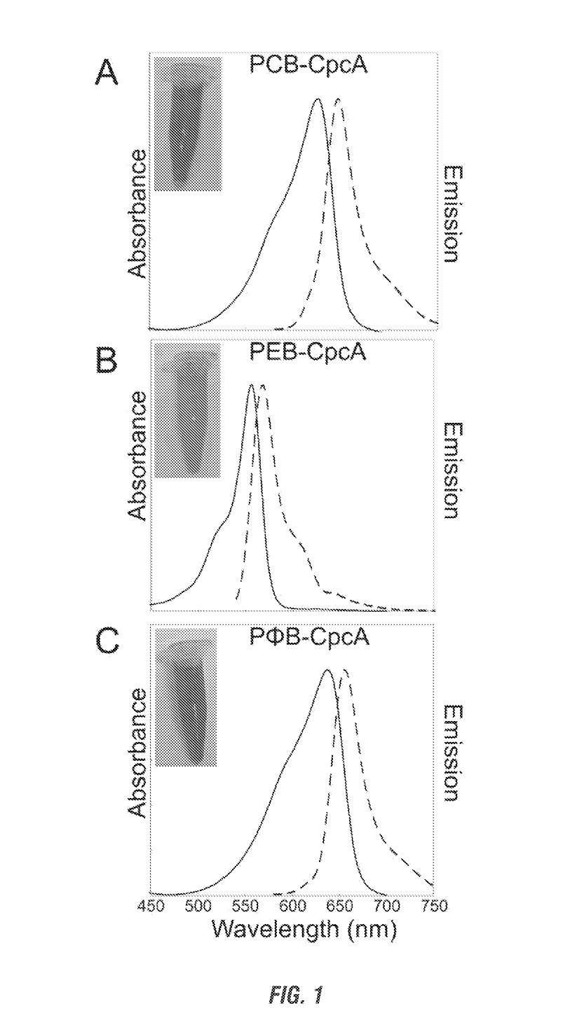

In order to determine if the CpcE/CpcF lyase can attach PEB to Cys82 of CpcA, E. coli BL21 (DE3) cells were co-transformed with plasmid pBS414V, containing the CpcA, CpcE, and CpcF genes, and pPebS, containing hox1 and pebS (Table 1). After IPTG induction and overnight incubation at 18° C., the E. coli cells were an intense red color. Upon affinity purification of the expressed HT-CpcA, absorption and fluorescence emission spectra were recorded to determine the effects of the co-expression of the alternative bilin synthesis enzymes (FIG. 1B). The results showed that the CpcE/CpcF lyase could attach PEB to apo-HT-CpcA. HT-CpcA-PEB had an absorption maximum at 556 nm and a fluorescence emission maximum at 568 nm. The protein was intensely fluorescent and had a quantum yield of 0.98. These properties are similar to those of naturally occurring phycoerythrins.

### Production of HT-CpcA-PΦB Using the Alternative Reductase, Phytochromobilin Synthase (HY2)

In order to determine if CpcE/CpcF was also capable of ligating PΦB to Cys82 of apo-HT-CpcA, E. coli BL21 (DE3) was co-transformed with pBS414v and pHY2, which encodes both Hox1 and HY2, which synthesizes PΦB. After induction and purification of the resulting HT-CpcA from the highly pigmented, greenish-blue colored cells, the absorption and fluorescence spectra of the protein were recorded (FIG. 1C). HT-CpcA-PΦB had an absorption maximum at 637 nm, a fluorescence emission maximum at 656 nm, and quantum yield of 0.18. These properties are similar to those of HT-CpcA-PCB except that both maxima are red-shifted by approximately 10 nm (FIGS. 1A and 1C; Table 2). This was expected because of the additional conjugated double bond contributed by the vinyl group on the D-ring of PΦB.

Co-Expression of CpcA with the Isomerizing PecE/PecF Lyase Resulted in Additional Chromophores not Previously Known to be Attached to CpcA

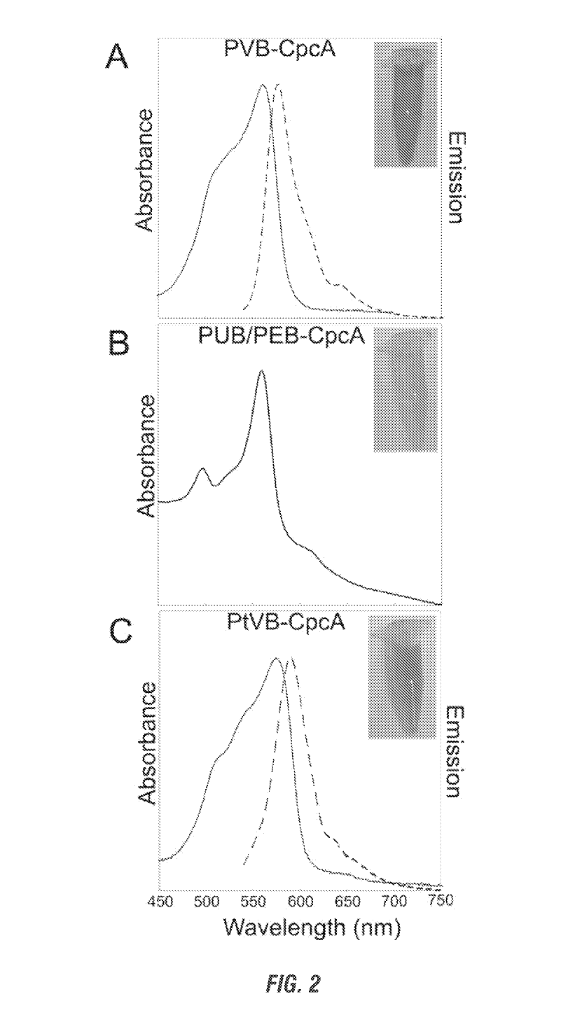

The results described above showed that the CpcE/CpcF lyase could attach PCB, PEB, and PΦB chromophores to HT-CpcA. These observations additionally suggested that apo-HT-CpcA has a bilin-binding site that could accommodate a variety of linear tetrapyrroles with different numbers of conjugated double bonds. In order to determine whether CpcA could bind other bilin chromophores, HT-CpcA was co-produced with the isomerizing lyase, PecE/PecF from Nostoc sp. PCC 7120. After IPTG induction and overnight incubation at 18° C., the E. coli cells expressing this combination of proteins were an intense, reddish-violet color. The PecE/PecF isomerizing lyase produced PVB from PCB and attached this isomerized chromophore to HT-CpcA (FIG. 2). HT-CpcA-PVB had an absorption maximum at 561 nm and a fluorescence emission maximum at 577 nm (FIG. 2A). The fluorescence quantum yield was only 0.14 (Table 2); this very low value was possibly due to the ability of the chromophore to undergo a light-induced isomerization (see below). These properties of HT-CpcA-PVB were very similar to those of native phycoerythrocyanin α subunits synthesized in cyanobacteria.

When the PecE/PecF lyase was produced together with HT-CpcA in a strain capable of synthesizing PEB (harboring pPebS; Table 1), the resulting E. coli cells were pale yellow-orange in color. Suggesting that a mixture of products was produced, the purified HT-CpcA protein from these cells had absorption maxima at 497 nm and at 560 nm. The former value is characteristic of PUB while the latter is similar to that observed for PEB as described above. Thus, the absorption spectrum suggested that both HT-CpcA-PUB and HT-CpcA-PEB were produced. When the PebS was replaced by the HY2 bilin reductase, the resulting E. coli cells were a pale violet color after IPTG induction and overnight incubation at 18° C. A new chromophore, which has not yet been described from natural sources, resulted from the isomerization of PΦB to what is here referred to as phytoviolobilin (PtVB). HT-CpcA-PtVB had an absorption maximum at 575 nm, a fluorescence emission maximum at 590 nm, and a fluorescence quantum yield of 0.23. PtVB is likely the same chromophore that was previously reported to be attached to PecA by PecE/PecF when PΦB was provided as a substrate in vitro as the resulting holo-protein was reported to have an absorbance maximum at 577 nm (35).

### The Attachment of Alternative Chromophores to HT-CpcA is Covalent

In order to verify that the bilins produced in E. coli were covalently bound to HT-CpcA, each of the purified proteins was subjected to analytical SDS-PAGE. After electrophoresis, the gel was soaked in ZnCl₂ to enhance the bilin fluorescence to visualize whether a bilin was covalently bound to the polypeptide. The gel was scanned using an excitation laser at 532 nm. The results are shown in FIG. 3 and they indicate that for each CpcA and chromophore combination, the bilin was covalently attached to the protein.

CpcA with PVB or PtVB is Photochemically Active

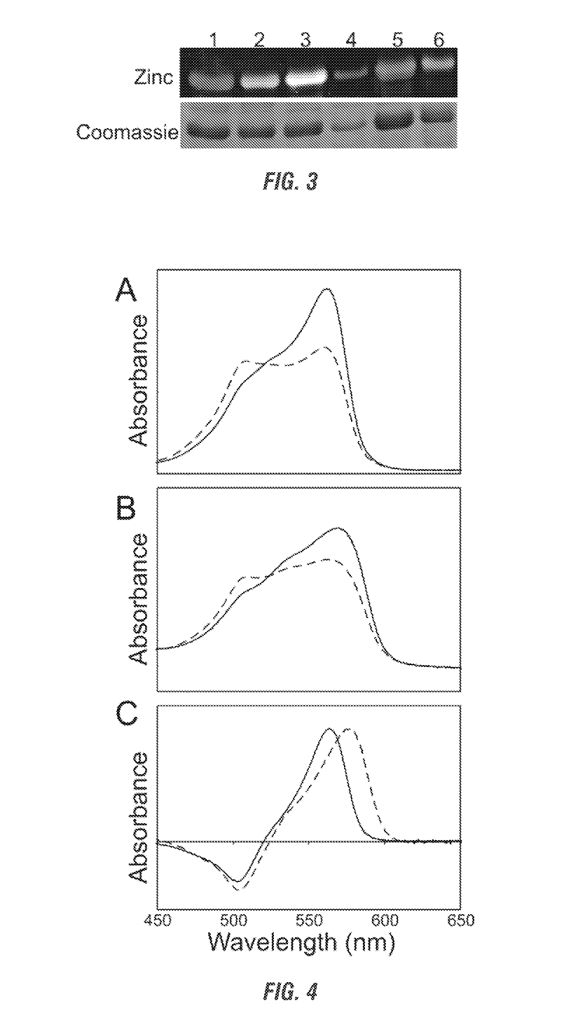

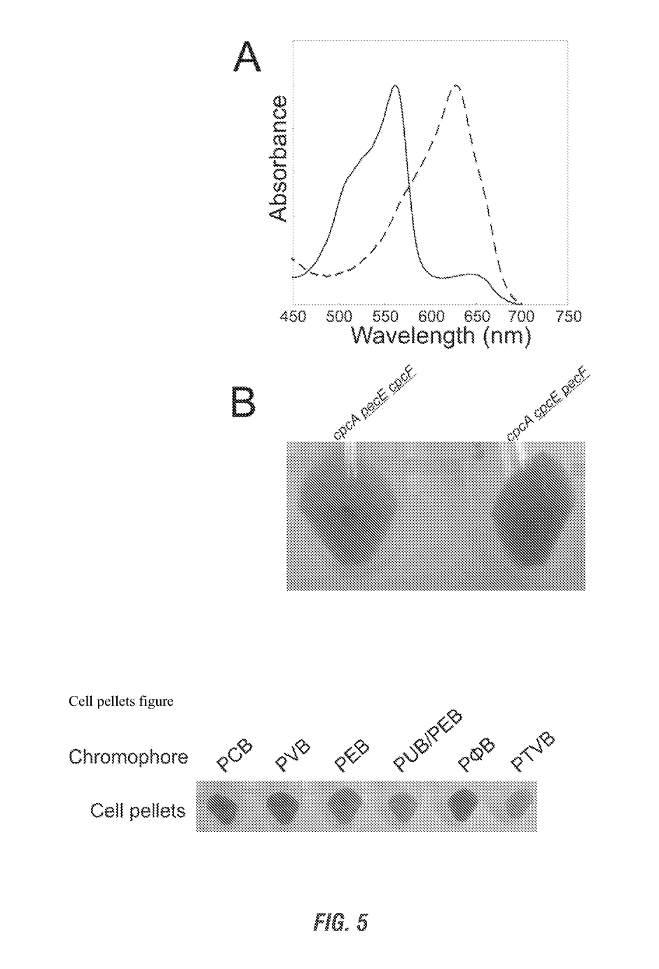

Naturally produced PecA (i.e., the α subunit of phycoerythrocyanin) has been shown to undergo fully reversible photochemistry similar to phytochrome-type photoreceptors. In order to determine if HT-CpcA-PVB, HT-CpcA-PΦB, and HT-CpcA-PtVB were similarly photoactive, samples of these proteins were exposed to saturating amounts of monochromatic light at their peak absorbance wavelengths for up to 2 h. After this incubation was complete, absorption spectra were immediately recorded to determine if any changes had occurred. If a light-induced change in the absorption profile had occurred, the photoreversibility of the change was tested by exposing the sample to saturating amounts of monochromatic light at the new absorption peak. The absorption spectrum of the resulting solution was similarly recorded and the results were compared. As shown in FIG. 4 both PVB-CpcA and PtVB-CpcA undergo reversible photoisomerization. However, no light-induced absorption changes were observed for HT-CpcA-PΦB (data not shown).

Chimeric Lyases Reveal the Isomerization Activity is Associated with PecF

The CpcE/CpcF and PecE/PecF lyases are clearly related by a gene duplication event, but only the latter is capable of both isomerizing and attaching chromophores to phycobiliproteins α subunits. To determine whether the isomerization activity is associated with the PecE or PecF subunits of the lyase, chimeric lyases PecE/CpcF and CpcE/PecF were produced in E. coli cells together with apo-HT-CpcA and PCB. Although the yields of chromophorylated HT-CpcA were lower with these chimeric lyases, sufficient phycobilin attachment occurred to answer the isomerization question definitively. The HT-CpcA produced together with the chimeric lyase PecE/CpcF had an absorption spectrum similar to the protein produced with CpcE/CpcF. This indicates that the protein carried a PCB chromophore that had not been isomerized. The HT-CpcA produced together with the chimeric lyase CpcE/PecF had an absorption spectrum very similar to that produced by PecE/PecF. This indicates that the protein carried a PVB chromophore, which is produced by isomerization of PCB. Thus, these results indicated that PecF is responsible for the isomerization activity associated with the PecE/PecF lyase. When only a single subunit of these dimeric lyases was co-produced with HT-CpcA, no lyase activity was observed (data not shown).

### Discussion

Although the CpcA used in these experiments was from a cyanobacterium that normally uses only PCB as a chromophore, this protein is capable of binding other chromophores. When the PEB producing pebS gene is used this yields proteins with absorbance and fluorescence maxima blue shifted by about 74 nm. In vitro assays have previously demonstrated that CpcE/CpcF has a preference for PCB over PEB in both binding affinity and catalysis rate (36), but in this heterologous system, CpcE/CpcF was nevertheless very efficient in adding PEB to HT-CpcA. As was seen with the phytofluors, CpcA carrying the PEB chromophore is also significantly more fluorescent. By our measurements these proteins had greater quantum yields than the phytofluors and may be more useful as biological labels. The phytofluors however have the advantage of autocatalytic chromophore attachment and would thus not require the co-expression of a lyase.

The ability of a phycobiliprotein to readily bind multiple chromophores seems to be primarily a characteristic of the alpha subunit. It has been reported that both PecA and RpcA also have the ability to bind alternative chromophores. The CpcS bilin lyase which is responsible for attaching PCB to cpcB, ApcA, ApcB, ApcF, and ApcD shows much lower activity when attaching PEB in a heterologous expression system (Schluchter et al., data not shown). In cyanobacterial cells that produce both PCB and PEB, a mistake by the CpcS or CpeS bilin lyase in attaching PEB at Cys-82 would mean that energy from PCB at β-Cys-153 or α-Cys-84 would not be transferred to PEB at β-Cys-82.

The mixed chromophore content and generally low yield of chromophorylated CpcA from the CpcA/pecE/pecF/pebS strain is a bit surprising. PEB attaches very well to CpcA in the CpcA/CpcEF/pebS system. However when the structure of PUB is compared to those of the other bilins here it is apparent that PUB is the most different from the native PCB chromophore due to conjugation changes at both the A ring and the D ring ends of the chromophore and in the presence of the 18-vinyl group. Maybe these changes together result in a chromophore that is less likely to be bound to CpcA due to perhaps less recognition by the apoprotein itself or by its cognate lyases.

Although our findings are novel for the Synechocystis sp. strain PCC 6803 CpcA, there are examples of phycobiliproteins that naturally bind alternative chromophores. This phenomenon occurs in the type IV chromatically adapting cyanobacteria. These strains adjust the ratio of PEB or PUB bound to a single PEII α subunit depending on whether they are grown in blue light or white light. This process is thought to be the result of a light regulated change in lyase or lyase/isomerase activity. Evidence of the mis-attachment chromophores comes from the finding that in a Nostoc sp. strain PCC 7120 pecEF mutant in there is a significant attachment of PCB to PecA which normally binds only PVB.

Previously reported in vitro experiments with PecA and its cognate lyase PecE/PecF suggested that PEB was not an acceptable chromophore substrate for this system. This has since been shown not to be the case when this system was expressed heterologously in E. coli. Our results confirm that the PecE/PecF lyase can use PEB as a substrate and extend this result by demonstrating that this lyase does not necessarily even require the native acceptor protein PecA.

PΦB synthesis has not been demonstrated to occur naturally in cyanobacteria. The reason for this divergence is unclear but PCB can be a functional chromophore for plant phytochromes and pcyA can largely complement a HY2 deficient mutant. Differing only in the presence of an extra vinyl group, it is not unexpected that these related chromophores can be used interchangeably. We have demonstrated here that CpcA can be readily synthesized with the PΦB chromophore. This yields a protein with a red shift in absorbance and fluorescence by about 11 nm though with a reduced quantum yield (Table 1).

The PecE/PecF isomerizing lyase has not been previously shown to be capable of attaching chromophores to CpcA. However, it has recently been shown that it can use RpcA as an acceptor substrate for the attachment of both PVB and PUB. That PecE/PecF is functional with HT-CpcA is somewhat surprising, however, because this protein in Synechocystis sp. strain PCC 6803 is never believed to carry these isomerized chromophores. In light of this it is not too surprising that these enzymes are also capable of attaching the similarly isomerized form of PΦB that we here call PtVB. Because HY2 is only known to occur in plants and PecE/PecF type isomerases are not known to occur in plants, PtVB is unlikely to be a naturally produced chromophore. Because PtVB differs from PVB in the same manner that PΦB differs from PCB, it was expected that the absorption and fluorescence emission maxima for PtVB-CpcA would be similarly red-shifted as for those of PVB-CpcA, and this was indeed the case.

It is only somewhat of a recent development that chromophore attachment to phycobiliproteins can be examined in a heterologous in vivo system. This appears to have many advantages over work that has previously been done in vitro, including the lack of mixed populations of proteins that have attached oxidized bilin products and the presence of subunits carrying non-covalently attached bilins in the chromophore-binding pocket. Based on the absorption and fluorescence profiles of these proteins, it appears that most of these preparations contain uniformly chromophorlyated products which are covalently binding primarily one type of bilin. The exception to this is the CpcA isolated from the CpcA/pecE/pecF/pebS strain which clearly a mixture of both HT-CpcA-PUB and HT-CpcA-PEB.

The majority of the work done to date on understanding the PecE/PecF lyase/isomerases has been done in vitro. It was only recently reported that PecE/PecF was capable of acting on PEB rather than just PCB and with RpcA as an acceptor rather than PecA (8). The results presented here extend our understanding of how this lyase works by showing that it can also utilize CpcA as an acceptor/substrate and that it can isomerize three different substrates: PCB, PEB, and PΦB. Previous studies, which showed a small (6%) residual isomerase activity for the PecF subunit alone in vitro with PecA and PCB as substrates, had suggested that the isomerase activity was associated with the PecF subunit of this isomerizing lyase. By forming chimeric lyases, it was unambiguously shown that CpcE/PecF attached PVB to HT-CpcA with virtually no other chromophorylated products being produced. We were unable to detect any lyase activity for PecF alone, and similar to other studies in vitro, CpcE and PecE also had no lyase activity in vivo. When CpcF was combined with PecE, the resulting chimeric lyase attached only PCB to HT-CpcA and little or no PVB was observed. The combination of these results establishes conclusively that the isomerization activity of the PecE/PecF lyase must reside with the PecF subunit.

TABLE 1
Plasmids used for this study

| | Recombinant proteins | | | |
|---|---|---|---|---|
| Plasmid Name | produced^{a} | Parent vector | Antibiotic^{b} | Reference |
| pPcyA | Synechocystis sp. PCC 6803 | pACYC Duet | Cm | Biswas et al., 2010 |
| | HO1 and Synechococcus sp. | | | |
| | PCC 7002 HT-PcyA | | | |
| pPebS | Myovirus HO1 and HT-PebS | pACYC Duet | Cm | Dammeyer et al., 2008 |
| pHY2 | Synechocystis sp. PCC 6803 | pACYC Duet | Cm | This paper |
| | HO1 and A. thaliana HT-HY2 | | | |
| pBS414v | Synechocystis sp. PCC 6803 | pBS350v | Sp | Tooley et al., 2001 |
| | HT-CpcA, CpcE and CpcF | | | |
| pBS405v | Synechocystis sp. PCC 6803 | pBS350v | Sp | Tooley et al., 2001 |
| | HT-CpcA | | | |
| pBS405vpecEF | Synechocystis sp. PCC 6803 | pBS405v | Sp | This paper |
| | HT-CpcA, Nostoc sp. PCC | | | |
| | 7120 PecE and PecF | | | |
| pBS405vpecF | Synechocystis sp. PCC 6803 | pBS405v | Sp | This paper |
| | HT-CpcA, Nostoc sp. PCC | | | |
| | 7120 PecF | | | |
| pBS405vpecE | Synechocystis sp. PCC 6803 | pBS405v | Sp | This paper |
| | HT-CpcA, Nostoc sp. PCC | | | |
| | 7120 PecE | | | |
| pBS405vcpcEpecF | Synechocystis sp. PCC 6803 | pBS405v | Sp | This paper |
| | HT-CpcA, CpcE and Nostoc | | | |
| | sp. PCC 7120 PecF | | | |
| pBS405vpecECpcF | Synechocystis sp. PCC 6803 | pBS405v | Sp | This paper |
| | HT-CpcA, CpcF and Nostoc sp. | | | |
| | PCC 7120 PecE | | | |
| pPL-PΦB | Synechocystis sp. PCC 6803 | pProLarA122 | Km | Fischer et al. 2005 |
| | HO1 and A. thaliana HY2 | | | |

Antibiotic resistance used to select for the presence of the plasmid (Cm: chloramphenicol; Km: kanamycin; Sp: spectinomycin)

TABLE 2
Chromophores attached to CpcA in this study and features of the resulting holoproteins

| | | Abs | Em | |
|---|---|---|---|---|
| Chromophore | Structure | Max | Max | QY |
| PCB | 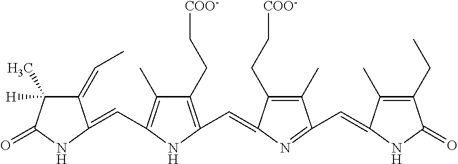 | 625 | 646 | 0.39 |
| PEB | 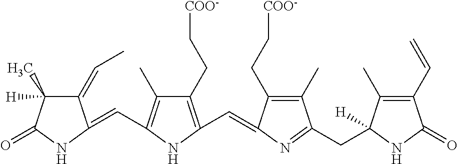 | 556 | 568 | 0.98 |
| PφB | 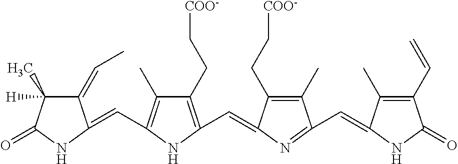 | 637 | 656 | 0.18 |
| PVB | 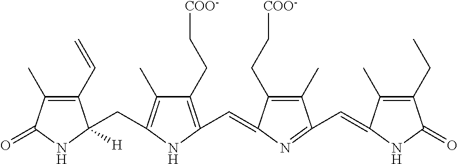 | 561 | 577 | 0.14 |
| PUB | 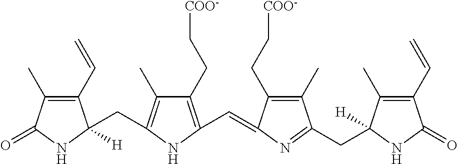 | 497 | ND | ND |
| PtVB | 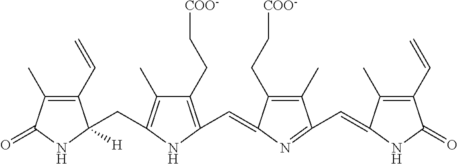 | 575 | 590 | 0.23 |

### Example 2

### Effects of Modified Phycobilin Biosynthesis in the Cyanobacterium Synechococcus sp. Strain PCC 7002

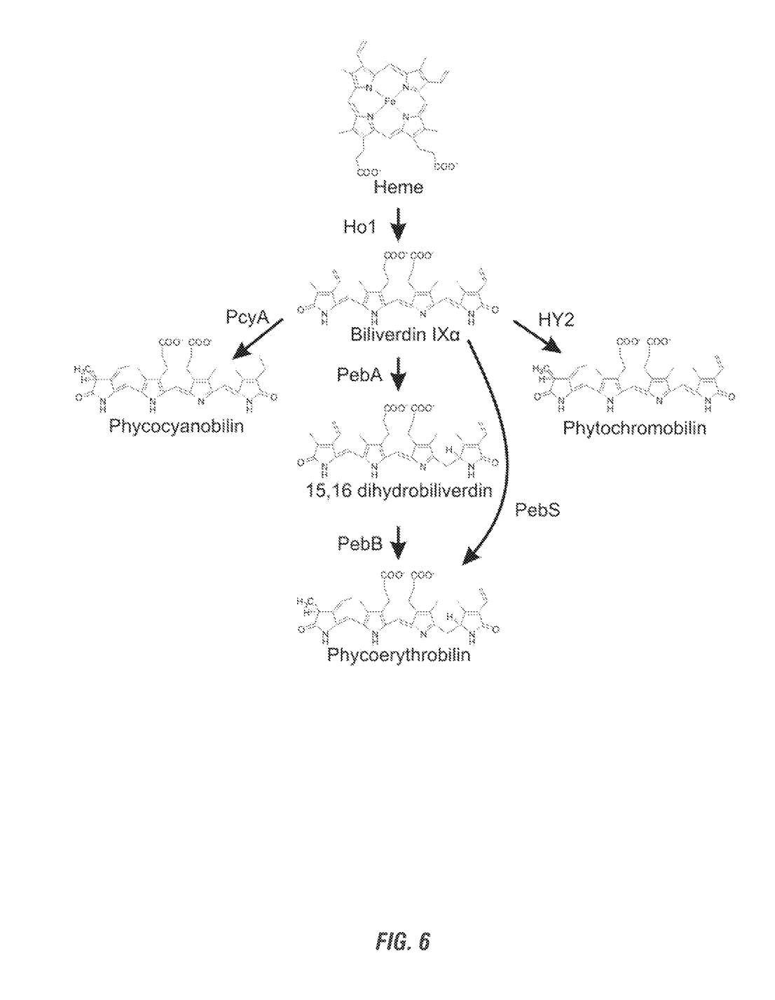

Most cyanobacteria employ light-harvesting antennae known as phycobilisomes (PBS) to collect light that is not efficiently absorbed by chlorophyll (Chl) for photosynthesis. PBS are, multi-subunit, supramolecular structures composed of both pigmented phycobiliproteins (PBPs) and usually non-pigmented linker proteins. Four different linear tetrapyrrole chromophores (bilins): phycocyanobilin (PCB), phycoerythrobilin (PEB), phycoviolobilin (PVB), and phycourobilin (PUB) can be bound to cyanobacterial PBPs. These four bilins are isomers, which differ only in the number of conjugated double bonds that form the chromophore, and all are derived from a common biosynthetic precursor, biliverdin IXα Biliverdin IXα is synthesized from heme by oxidative cleavage of a methine bridge of heme by the enzyme heme oxygenase. PCB:ferredoxin oxidoreductase, PcyA, uses four electrons from reduced ferredoxin to synthesize PCB by regiospecific reduction of the exo vinyl group of ring D and the endo vinyl group of ring A of biliverdin IXα. The reactions leading to the conversion of heme into biliverdin IXα, PCB and PEB are shown in FIG. 6. PEB is synthesized in a similar manner from biliverdin IXα by the sequential actions of two reductases, PebA and PebB, or the more recently discovered viral enzyme PebS, which alone catalyzes the same reactions performed by PebA and PebB (FIG. 6,). The remaining two chromophores, PUB and PVB, are synthesized from PEB and PCB, respectively, by isomerizing lyases that both isomerize the parental chromophores and attach them to their cognate PBP subunits.

Various species of cyanobacteria utilize different combinations of chromophores and PBPs to optimize their light harvesting for photosynthesis. This is generally believed to be due to adaptations to the specific light conditions available to a given species in its natural environment. As suggested by the phylum name, the archetypical cyanobacterium is blue-green in color and synthesizes Chl a and PBSs containing only the blue-colored (orange-absorbing) phycocyanin (PC) and aqua-colored (red-absorbing) allophycocyanin (AP), both of which carry PCB as the only type of chromophore. A few cyanobacteria can synthesize phycoerythrocyanin, a fuchsia-colored protein whose alpha subunit carries a single PVB chromophore. Still other cyanobacteria, many of which are marine organisms, can produce phycoerythrin (PE), a red-colored (green-absorbing) protein that carries PEB and sometimes PUB chromophores. Thus, proteins incorporating PCB have the most red-shifted absorption maxima while those with PUB have the most blue-shifted maxima. Many cyanobacteria also possess the ability to adjust their spectral profile to absorb the light available in their environment optimally. Some PE-producing species do this by coordinately regulating apoproteins, lyases, and chromophore production, as in type II or type III complementary chromatic acclimation. Other species, such as the type IV chromatically acclimating species, adjust their spectral profile to their light environment by simply regulating specific lyases to produce an isomerizing or non-isomerizing lyase, which then dictates the conjugation state and thus the absorption properties of an attached chromophore.

Phytochromobilin (PΦB), the chromophore of the higher plant photosensory proteins known as phytochromes, is very similar in structure to PCB, but no cyanobacterium has been shown to synthesize this bilin naturally. PΦB is more oxidized than PCB, differs from PCB only by the presence of a vinyl group at the C18 position, and shares the same biliverdin IXα precursor as PCB and PEB (FIG. 6). Recent studies show that PΦB can be covalently bound to CpcA by the cognate CpcE/CpcF lyase when heme oxygenase, PΦB synthetase (HY2), CpcA, and the CpcE/CpcF lyases are produced heterologously in Escherichia coli (Alvey et al, manuscript in preparation). The observation that CpcA from a cyanobacterium that normally only synthesizes PCB could bind alternative chromophores when produced in an appropriate heterologous expression system prompted us to explore the effects of changing the chromophore content “in-cyano” by genetically modifying Synechococcus sp. strain PCC 7002, which naturally only produces PCB, to produce other chromophores (viz., PEB and PΦB).

### Materials and Methods

### Cyanobacterial Growth Conditions

Medium A⁺ (medium A containing 12 mM Na-nitrate) was routinely used to grow wild-type Synechococcus sp. strain PCC 7002 cultures and the ΔpcyA::aadA strain producing HY2 for growth rate comparisons (33). Unless otherwise specified, cells were grown under standard conditions, which are 38° C. at 250 μmol photons m⁻² s⁻¹ and were sparged with air supplemented with 1% (v/v) CO₂. A glycerol-tolerant strain of Synechococcus sp. strain PCC 7002 was generated through the continuous growth on the wild-type strain in liquid A⁺ medium containing 10 mM glycerol and was used as a background for the generation of the pebAB and HY2 overexpression strains. Spectinomycin at 50 μg ml⁻¹ and gentamycin at 50 μg ml⁻¹ were added to media as needed for selection and maintenance of transformants.

### Construction of the Bilin Biosynthesis Mutants

To inactivate the pcyA gene of Synechococcus sp. strain PCC 7002, regions of approximately 600 bp immediately upstream and downstream of the pcyA gene were amplified by PCR by using primers pcyAR1F (SEQ ID NO:7) (5′-GCACTGATGCATATTCTGTGTGGACATCGTAGC-3′, NsiI site underlined) and pcyaR1R (SEQ ID NO:8) (5′-GGGCAGCCATGGGCGAAAAAGCAATCTTAA-3′, NcoI site underlined) for the upstream sequence and pcyAR2F (SEQ ID NO:9) (5′-TATTCCCCATGGGTCGACGAACAAAGCTTTAATTACCGCAG-3′, NcoI and SalI sites underlined) and pcyAR2R (SEQ ID NO:10) (5′-ACCTGAGCATGCTTCAGCCGACCCCTACCGA-3′, SphI site underlined) for the downstream sequence. These fragments were digested with NcoI and ligated together. The resulting product was digested with SphI and NsiI and ligated into pGEM-7Zf(+) (Promega Corporation, Madison, Wis.) to make pGEM-pcyAR1R2. The aadA cassette from pSRA81 was amplified by PCR with primers pst-aadAF (SEQ ID NO:11) (5′-GGTGCTCCATGGGGCTGCTAACAAAGCCCGAAA-3′, NcoI site underlined) and sal-aadAR (SEQ ID NO:12) (5′-GGAGGCGTCGACCTAGAGTCGAGCGAATTGTTAG-3′, SalI site underlined), and the resulting product was digested with NcoI and SalI and cloned into similarly digested pGEM-pcyAR1R2. The resulting plasmid was used as a template in a PCR reaction using primers pcyAR1F and pcyAR2R to create a 2.3-kb linear DNA fragment for natural transformation of a glycerol-adapted strain of Synechococcus sp. strain PCC 7002. The primers pcyAR1F and pcyAR2R were also used to assess segregation of the pcyA and pcyA::aadA alleles at the pcyA locus.

To introduce pebAB genes into plasmid pAQ1 of Synechococcus sp. strain PCC 7002, the pebAB genes from Synechococcus sp. strain PCC 7335 were amplified by PCR with primers 7335pebAF (SEQ ID NO:13) (5′-GCTGTGTCATATGTATCGCCCTTTTCAAGCG-3′, NdeI sited underlined) and 7335pebBR (SEQ ID NO:14) (5′-ATATTGAGGATCCCTAGGCAGCGATGCGAGCG-3′, BamHI site underlined). The resulting amplicon was digested with NdeI and BamHI and cloned into similarly digested pAQ1cpcEx which allows for the convenient cloning and introduction of foreign genes driven by the cpcBA promoter of Synechocystis sp. strain PCC 6803 into the endogenous pAQ1 plasmid of Synechococcus sp. strain PCC 7002.

The HY2 gene was also expressed on endogenous plasmid pAQ1 under the control of the Synechocystis sp. PCC 6803 cpcBA promoter; however, due to the presence of most convenient restriction sites, in particular NdeI, NcoI and BamHI, in the HY2 gene itself, the pAQ1cpcEx vector was modified to facilitate the cloning of this gene. Firstly, the gentamycin cassette from pMS266 was amplified using primers gentF: (SEQ ID NO:15) (5′-ATCCGTTCTAGAAGGTCTTCCGATCTCCTGAAG-3′, XbaI site underlined) and gentR (SEQ ID NO:16): (5′-CCTAGAGTCGACGTCGGCCGGGAAGCCGAT-3′, SalI site underlined). The resulting product was digested with XbaI and SalI and cloned into pAQ1cpcEx that had also been digested with XbaI and SalI. This switched the drug resistance cartridge from aadA, which confers spectinomycin resistance to aacC1, which confers gentamycin resistance. In order to replace the BamHI site on this vector with PstI, a region including this new cassette was amplified by PCR with primers paq1fix (SEQ ID NO:17): (5′-GGTGCT CCATGG CTGCAG GGCTGCTAACAAAGCCCGAAA-3′, NcoI and PstI sites underlined) and gentR. This product was then digested with NcoI and SalI and cloned into pAQ1cpcEx that had been similarly digested. The HY2 gene was amplified by PCR from plasmid pPL-PΦB using the primers bspHY2F (SEQ ID NO:18) (5′-CCCTGGTCATGATCTCTGCTGTGTCGTATAAGG-3′, BspHI site underlined) and KOHy2R (SEQ ID NO:19) (5′-TATGCTGCAGTTAGCCGATAAATTGTCCTGTTAAATCCCAAGGACGAG-3′, PstI site underlined) and cut with BspHI and PstI and cloned into this modified pAQ1cpcEx vector which was cut with NcoI and PstI.

To create a linear DNA fragment for natural transformation into Synechococcus sp. strain PCC 7002, the pebAB and HY2 plasmids were used as templates in PCR reactions with the primers pAQ1sph (SEQ ID NO:20) (5′-CCCGACGTCGCATGCCTC-3′) and pAQ1nsi (SEQ ID NO:21) (5′-GAATACTCAAGCTATGCATGGG-3′).

Spectroscopic and Compositional Analyses of Bilin Biosynthesis Mutants and their PBS

Absorption spectra of whole cells of Synechococcus sp. strain PCC 7002 and its isolated PBS were recorded with a GENESYS 10 spectrophotometer (ThermoSpectronic, Rochester, N.Y.).

Chl a was extracted using 100% methanol, and concentrations were determined according to previously described methods. The relative PBP contents of cells were determined using heat-induced bleaching at 65° C. as described previously except that the method was modified to examine the peak absorption of the PBSs, which because of the red-shifted absorbance of the PΦB chromophore caused the PC peak to differ between the two strains.

PBSs were isolated as described previously, and their protein contents were analyzed by polyacrylamide gel electrophoresis (PAGE) in the presence of sodium dodecylsulfate (SDS). Proteins separated on a 14% (w/v) polyacrylamide gel were first stained with a 100 mM ZnCl₂ solution to enhance the fluorescence from the bilins (6), which was visualized using a Typhoon 8600 Variable Mode Imager (GE Healthcare Lifesciences, Pittsburgh, Pa.). Total proteins were then stained with Coomassie blue and imaged with an Epson Perfection V750 flatbed scanner. His-tagged preparations of Synechocystis sp. strain PCC 6803 holo-CpcA, chromophorylated with either PCB or PEB, were used for comparison in these experiments. These recombinant proteins were heterologously expressed in E. coli BL21(DE3) and were isolated as described previously.

Fluorescence measurements of whole cells and isolated PBS were taken using a SLM 8000C spectrofluorometer modernized for computerized data acquisition by On-Line Systems, Inc. (Bogart, Ga.). Whole-cell fluorescence at 77 K was measured by resuspending cells in 50 mM HEPES/NaOH buffer, pH 7.0 containing 60% (v/v) glycerol and freezing the mixtures in liquid nitrogen as described previously. The excitation wavelength was 590 nm for excitation of PBS and PBPs.

Prior to high performance liquid chromatography (HPLC) separation, the isolated PBP were dialyzed against 10 mM sodium phosphate, pH 7.0, 1 mM 2-mercaptoethanol. Samples were diluted 1:1 with 6 M guanidium hydrochloride, pH 5.2, and centrifuged for 5 min prior to injection. The HPLC chromatography was performed using Waters (Waters Chromatography Division, Milford, Mass.) Model 510 pumps and Model 600 automated gradient controller. Data were acquired using Waters 2996 photodiode array detector; spectra were collected from 220 to 700 nm at 1-s intervals.

### Results

Attempted Insertional Inactivation of pcyA in Wild-Type Synechococcus Sp. Strain PCC 7002.

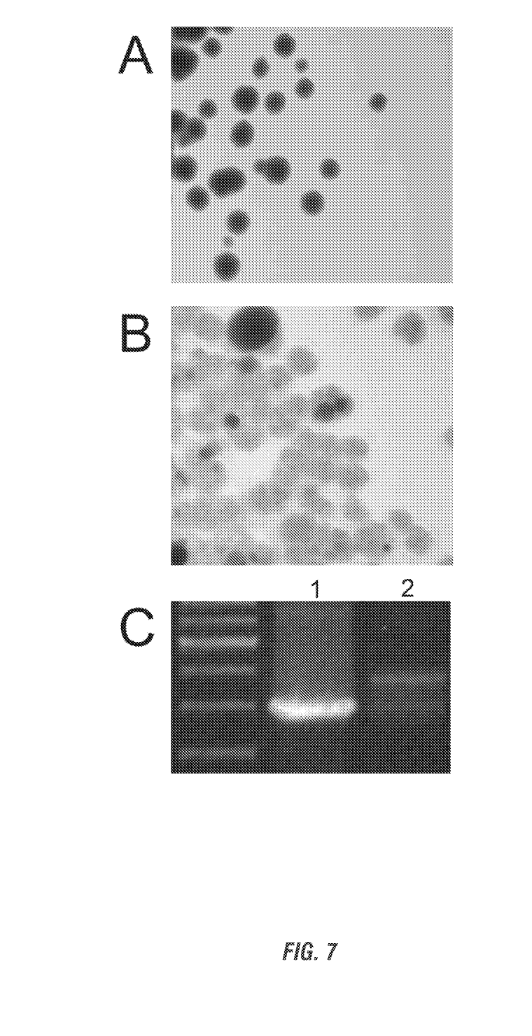

In order to gain a better understanding of the dual role of PCB in both light harvesting and light sensing in Synechococcus sp. strain PCC 7002, an attempt was made to inactivate pcyA insertionally. As for other mutations that could potentially affect the photosynthetic capacity of this cyanobacterium, constructions to inactivate the pcyA gene were transformed into a glycerol-tolerant strain that could grow photomixotrophically on glycerol if the mutation in question caused unexpected problems for photosynthesis. Similar conditions were previously employed to produce mutants lacking PC and AP in this cyanobacterium. Colonies from the resulting transformation with the pcyA::aadA construct were initially indistinguishable from wild-type Synechococcus sp. strain PCC 7002 (FIG. 7A). However, upon several rounds of selection, the majority of the colonies began to be yellow-green (chlorotic) in color, which was consistent with an inability to synthesize PBPs (FIG. 7B). Despite continued careful selection of colonies lacking the dark, blue-green pigmentation of the wild type, continued sectoring suggested the persistence of wild-type copies of pcyA in the genomes of transformed cells. PCR analyses confirmed the presence of both pcyA and pcyA::aadA alleles within the population (FIG. 7C), and full segregation of the pcyA and pcyA::aadA alleles was never observed. These observations suggest that under standard photoheterotrophic growth conditions the pcyA gene of Synechococcus sp. strain PCC 7002 is essential.

Overexpression of pebAB in Synechococcus Sp. PCC 7002 Produces a Readily Visible but Unstable Phenotype.

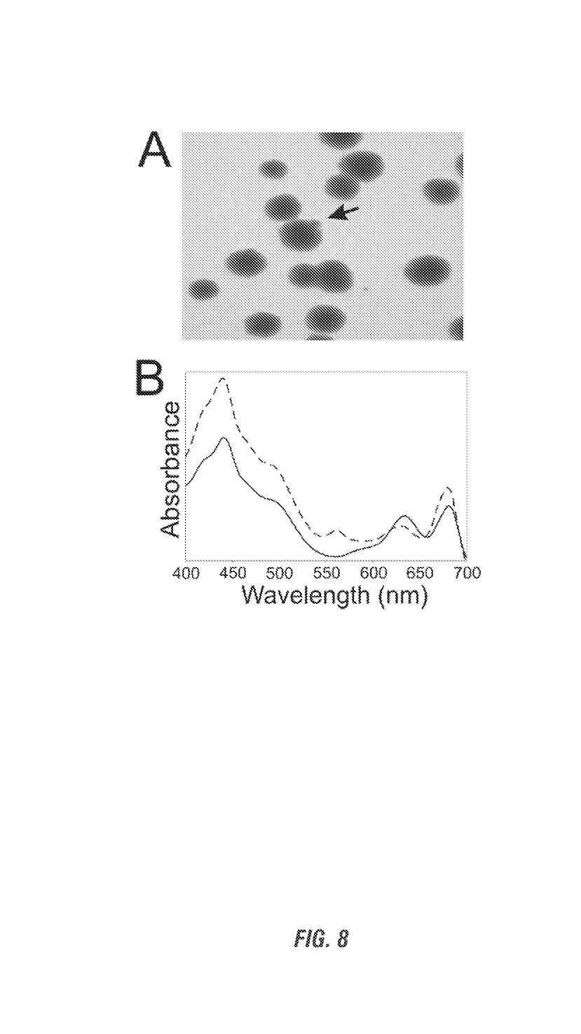

PEB can readily be attached to CpcA by CpcE/CpcF lyase when all necessary proteins are heterologously expressed in E. coli. This observation prompted a study to determine if this phenomenon could be replicated in a cyanobacterium, in which chromophorylation might be more tightly controlled because of the potential to cause deleterious effects on the photosynthetic capacities of cells. For this experiment, pebA and pebB, encoding a 15,16 dihydrobiliverdin oxidoreductase and the phycoerythrobilin reductase, respectively, from the chromatically acclimating cyanobacterium Synechococcus sp. strain PCC 7335 were placed under the control of the strong cpcBA promoter of Synechocystis sp. strain PCC 6803 and introduced into a neutral site on the endogenous pAQ1 plasmid of Synechococcus sp. strain PCC 7002 (37). This construct was transformed into a glycerol-tolerant strain, which was grown mixotrophically with glycerol. As for the transformants containing the pcyA deletion construct, transformants possessing the pebAB construct were initially indistinguishable from the wild type. However, after several successive rounds of selection and restreaking, colonies began to show regions with brownish to brick red color. By careful selection of colonies, it was eventually possible to isolate nearly uniformly reddish-brown colonies that resembled the colonies of strains that naturally produce phycoerythrin, although complete elimination of colonies with the blue-green coloration of the wild-type strain was never completely achieved (FIG. 8A). The absorption spectrum of a typical transformant strain (FIG. 8B) revealed an additional absorption peak at about 556 nm (the absorption maximum of PE) and an apparent decrease in the relative absorption of PC at ˜635 nm.

To assess the PBP composition of cells overproducing PebA and PebB, intact PBS were isolated. In order to have sufficient cell material for analysis, liquid cultures were started with a relatively high amount of starter inoculum and grown for a relatively short period (˜40 h). Smaller starter inocula and/or longer incubation times tended to favor the appearance of revertants that resembled wild-type cells and lacked brown pigmentation. This might have been due to the accumulation of mutations that could inactivate the expression of pebA and pebB, which would allow more rapidly growing wild-type cells to predominate. In spite of the instability of the genotype/phenotype, by keeping the inocula large and the incubation times minimal, it was possible to grow cells that retained most of the phenotype that was visible on plates (FIG. 8A).

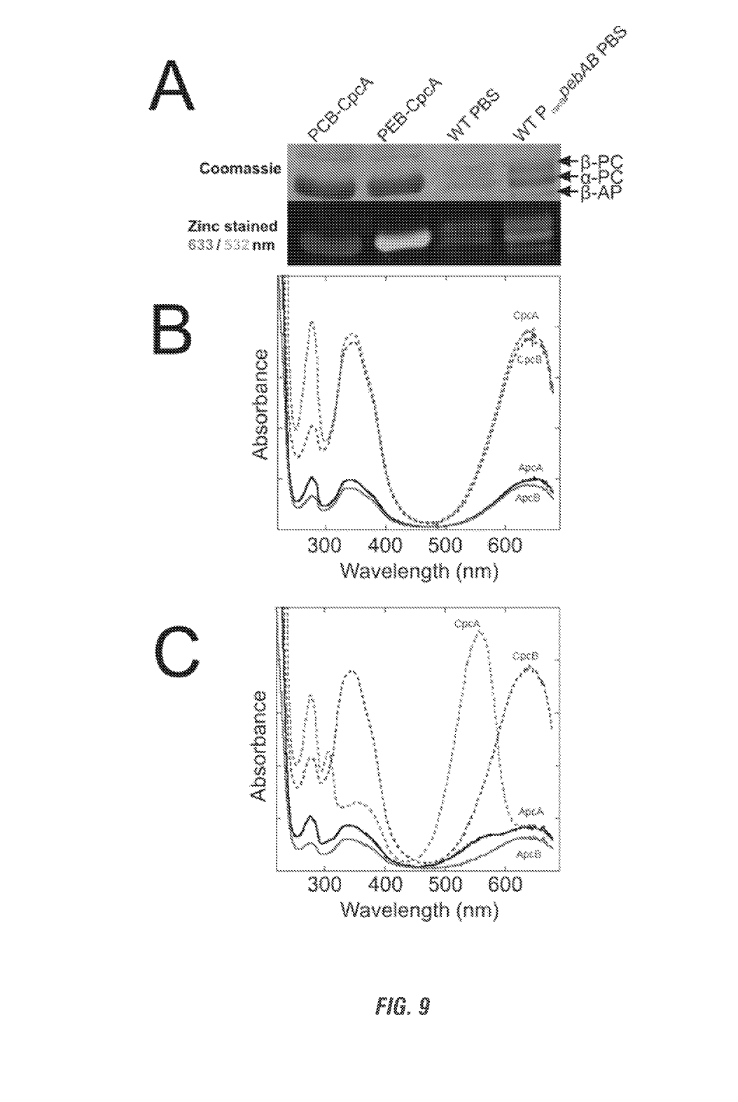

In order to examine the distribution of PEB among the major PBPs isolated from PBS isolated from the pebA pebB and wild-type strains, PBPs were separated by analytical SDS-PAGE alongside aliquots of purified recombinant [His]₆-CpcA carrying PCB or PEB. The proteins were detected by both zinc-enhanced fluorescence and by staining with Coomassie blue (FIG. 9A). Using excitation lasers at 532 nm and 633 nm, the zinc-enhanced gel was imaged by fluorescence on a Typhoon 8600 Variable Mode Imager. The results were compared to those for proteins with known chromophore content to assess qualitatively which polypeptides carried PEB chromophores in the PBSs isolated from cells overproducing PebA and PebB. The PC α subunit (i.e., CpcA) was significantly more fluorescent when scanned with the 532 nm laser than was the case for CpcA from the wild-type strain, and the intensity of its fluorescence was similar to that of the recombinant [His]₆-CpcA-PEB protein standard. These same PBP samples were subsequently analyzed by HPLC in order to confirm these results and to determine if PEB was incorporated into any of the other PBS components. When the absorbance profiles of the major PBP components of PBS isolated from the pebA pebB overexpression strain (FIG. 9C) were compared to those from the wild-type strain (FIG. 4B), the only PBP subunit that exhibited a major difference in absorption was the phycocyanin α subunit. A very small amount of PEB was also observed for the ApcA subunit, but little or no PEB was apparently incorporated into the phycocyanin 0 subunit (CpcB). These results suggested that very little PEB was incorporated into other PBP subunits that could be assembled into PBS.

pcyA can be Inactivated in a Strain Expressing HY2.

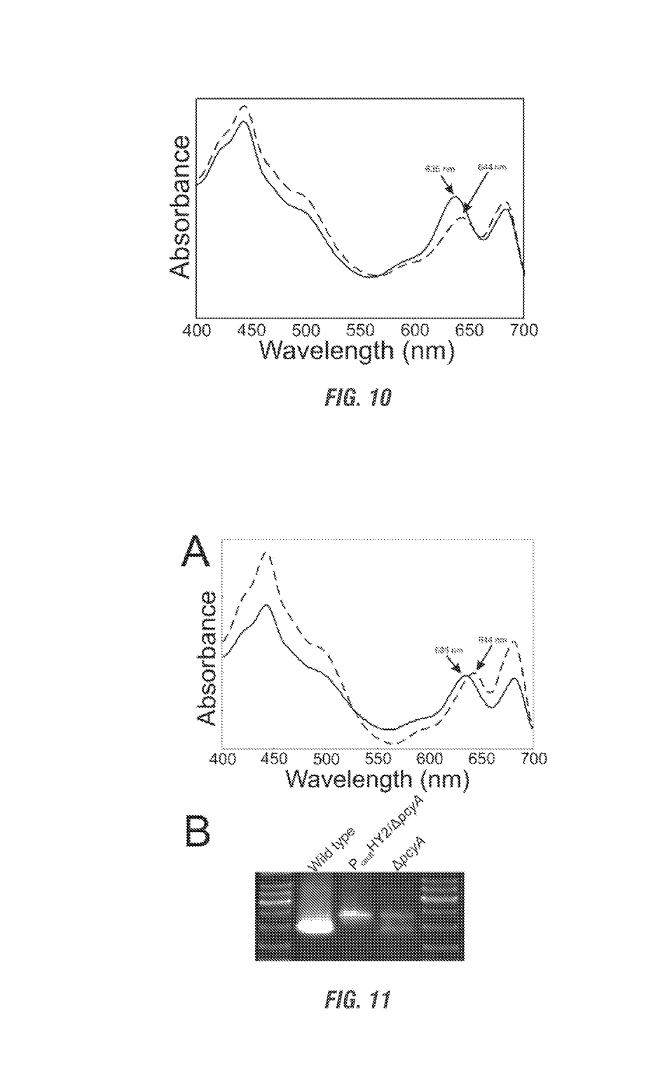

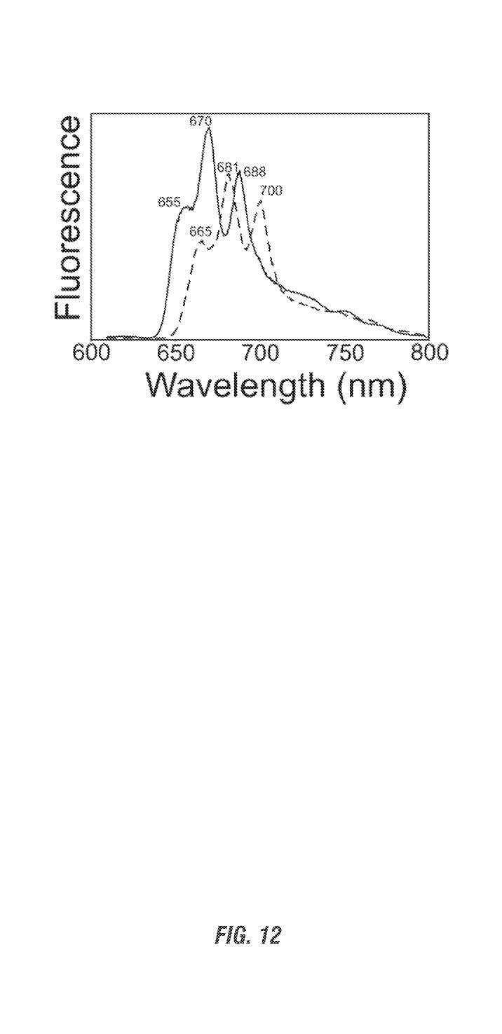

In addition to being able to bind PEB when produced in a heterologous expression system, CpcA was also capable of binding PΦB (R. Alvey, manuscript in preparation). The Arabidopsis thaliana HY2 gene was introduced into plasmid pAQ1 of Synechococcus sp. strain PCC 7002 under the control of the very strong cpcBA promoter of Synechocystis sp. strain PCC 6803. Strains expressing HY2 appeared slightly less blue and thus slightly greener than the WT (data not shown). An examination of the absorption spectra for this strain revealed that the absorption maximum for PC was red-shifted by approximately 10 nm (FIG. 10). Absorption shifts of similar magnitude, about 10 nm to shorter wavelength, have typically been observed when phytochromes have been reconstituted with PCB instead of PΦB. Because of the very high expression levels of HY2 expected when driven by the cpcBA promoter, and the similarity of the resulting PΦB chromophore to the native PCB chromophore, it seemed possible that the large proportion of the PBPs in these cells might carry PΦB and not PCB chromophores. Experiments in plants have previously shown the two chromophores to be nearly interchangeable. Because this strain carried functional pcyA and HY2 genes, it was possible that sufficient PCB was available to allow the selective assembly of some PBPs with PCB, as was seen for the strain overexpressing pebA and pebB.

In order to determine if PΦB could serve as the only bilin chromophore for Synechococcus sp. strain PCC 7002, cells overproducing HY2 were transformed with the same DNA construct, pcyA::aadA, previously used unsuccessfully to inactivate pcyA in the wild-type background. In contrast to the previous results with the wild type, the transformant colonies in which pcyA was inactivated in the HY2 over-expression background did not develop a chlorotic appearance and segregation of the mutant and wild-type alleles was rapidly achieved (FIG. 11B). The absorption spectrum of the resulting strain was very similar to that of the background strain (FIGS. 10 and 11A). However, because no wild-type alleles of pcyA could be detected by PCR, it can be inferred that all of the PBPs produced in this strain must carry PΦB rather than PCB chromophores.

Fluorescence Emission is Shifted in the HY2 Expressing pcyA Deletion Strain.

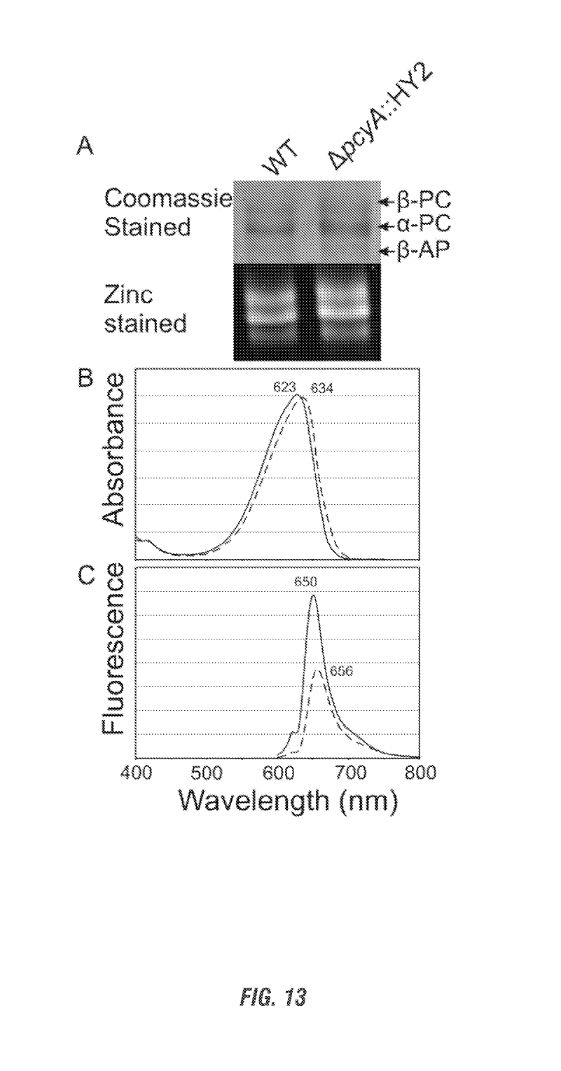

In order to examine the effects of global changes in the chromophore composition of PBSs, low-temperature (77 K) fluorescence emission spectra of cells were measured. Spectra for both the wild type and the strain overexpressing HY2 in a pcyA deletion background were recorded with the excitation wavelength set to 590 nm to excite mainly the PBPs. For the wild type, maxima at ˜645 nm, ˜660 nm and ˜680 nm represent emission from PC, AP, and the terminal emitters ApcD and ApcE, respectively. As expected, based on the absorption profile of the ΔpcyA::HY2 strain, the replacement of all PCB chromophores with PΦB caused a red shift in the fluorescence emission of the PBPs in this strain. Fluorescence emission peaks for PC, AP, and ApcD/ApcE each shifted about 15 nm, with the new maxima occurring at 657 nm, 674 nm, and 693 nm, respectively. Although the intensity of fluorescence was lower for the mutant strain, the intensities of the three peaks relative to each other remained similar for the mutant strain. This observation suggested there was no significant change in the relative amounts of PC, AP, and terminal emitters in the PBS of the mutant strain. This was further corroborated by SDS-PAGE analyses of the PBS from the mutant strain (FIG. 13A). Zinc staining of the gel showed similar levels of chromophore content and similar relative abundances of the PBS components in the two strains (FIG. 13A). As expected, the absorbance and fluorescence of the isolated PBS was red-shifted (FIGS. 13B and 13C). PBS assembled from PBPs carrying PΦB also appeared to be much less fluorescent than those produced from proteins carrying PCB (FIG. 13C). Comparisons of the PBP and chlorophyll contents of wild type and the ΔpcyA::HY2 mutant strain revealed that the mutant strain contained about 40% less PBPs than the wild type while the chlorophyll contents of the two strains remained similar (data not shown). Taken together these observations suggested that, not only are the PBPs less abundant in the ΔpcyA::HY2 mutant, but they are also less effective in the transmission of absorbed light energy. Thus, this strain should have growth defects due to impaired light harvesting.

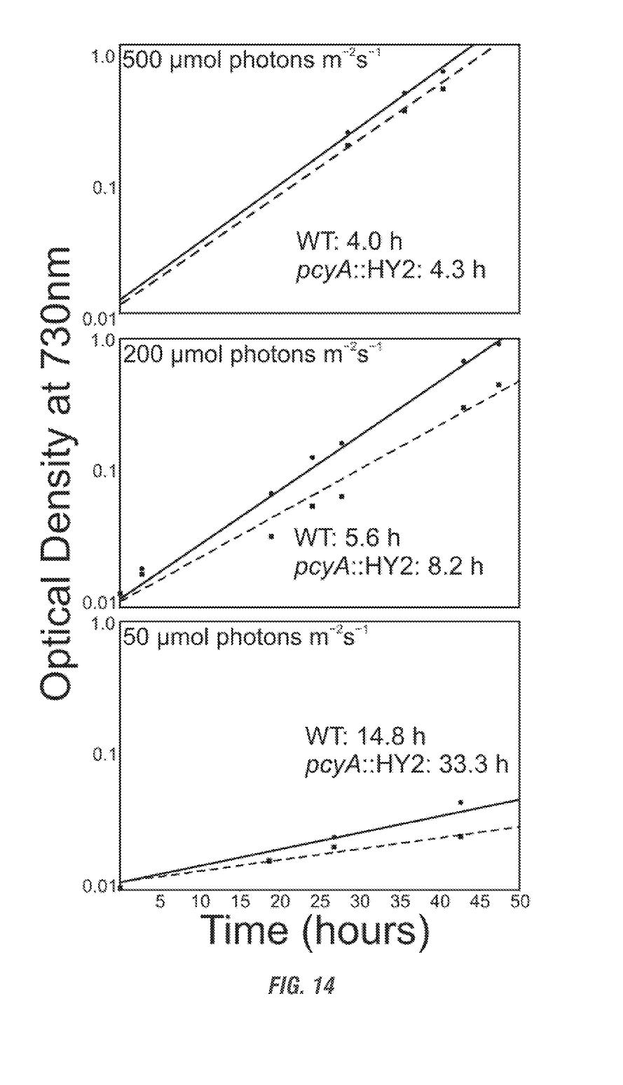

The apparent requirement for either PCB or PΦB chromophores by Synechococcus sp. strain PCC 7002 allowed for the cultivation and further extensive characterization of this strain in the absence of the antibiotics marker normally used to select for strains harboring the recombinant DNA constructs. Growth rate measurements were conducted to determine the effects of the altered bilin content on the light-harvesting capabilities of this strain. Growth rates were determined using the standard growth conditions for of Synechococcus sp. strain PCC 7002 (38° C., 1% (v/v) CO₂ in air, nitrate as N-source) at three different light intensities 50, 200, and 500 μmol photons m⁻² s⁻¹ (FIG. 14). When light was limiting (50 μmol photons m⁻² s⁻¹), the doubling time for the ΔpcyA::HY2 strain was more than 2-fold longer than that of the wild type. At an intensity slightly less than saturating (200 μmol photons m⁻² s⁻¹), the doubling time for the ΔpcyA::HY2 strain was about 50% longer than the wild type. At a supra-saturating light intensity (500 μmol photons m⁻² s⁻¹), the growth rates for the two strains were virtually indistinguishable. These data showed that the ΔpcyA::HY2 strain was significantly impaired in light energy harvesting in comparison to the wild type.

### Discussion

The inability to inactivate the pcyA gene in the genome of Synechococcus sp. strain PCC 7002 suggested that PcyA, and by extension its product, PCB, are required for cellular viability. This observation was surprising for several reasons. Previous studies had shown that it is possible to produce strains in which the genes for cpcBA and apcAB had been deleted, and the double mutant does not accumulate detectable levels of PBPs. In those studies, however, the apo-PBPs were not synthesized, and it remains a possibility that the additional back-selection pressure for retention of the capacity to synthesize the antenna prevented complete segregation of the pcyA and pcyA::aadA alleles in the experiments described here. An alternative explanation for these observations might be that there are other proteins that require PCB chromophores that are essential when cells are grown in continuous illumination. Candidates for such proteins include phytochrome and cyanobacteriochrome-like proteins encoded in the Synechococcus sp. strain PCC 7002 genome. While several of these are homologous to proteins characterized in Syncechocystis sp. PCC 6803, neither their potential cognate chromophore nor the phenotype of mutants for these genes has been investigated. The requirement for PCB for purposes other than the chromophore for phycobiliproteins is further supported by the recent sequencing of the genome of the cyanobacterium UCYN-A, a marine cyanobacterium with a highly reduced genome. Although this genome does not encode any PBPs, it has retained the pcyA gene. It has also retained several GAF domain proteins that may serve photosensory functions. Further attempts to inactivate the pcyA gene when cells are grown heterotrophically in the dark are currently in progress to address the possibility that light produces the effects observed.

High-level expression of pebAB, in the absence of expression of PE apo-proteins or associated linker polypeptides, nevertheless produced a whole-cell absorption spectrum, which resembled that of a PE-producing cyanobacterium (FIG. 8). Strains with this phenotype were extremely unstable, however, and could only be maintained by carefully choosing the most reddish-brown colonies, and even with this precaution, apparent phenotypic revertants (to blue-green coloration) were common (FIG. 8A). When cells were cultivated for more than about two days, liquid cultures rapidly reverted to wild-type pigmentation and absorption. One explanation for this is that hyper-expression of pebA and pebB might starve the culture for PCB. Because PcyA and PebA utilize the same substrate, biliverdin IXα, severe over-expression of pebA could limit the availability of PCB, which as noted above appears to be required for viability when cells are grown in continuous light. Although PCB in principle should still be produced, high levels of PEB might competitively inhibit reactions in which PCB is the natural substrate. Previous in vitro experiments using CpcA with it its cognate CpcEF lyase have demonstrated that PEB can function as a competitive inhibitor for PCB addition. If PCB is required to produce a functional phytochrome-like molecule, PEB would probably be unable to satisfy this requirement, because it lacks the 15-16 double bond present in PCB that is the site of reversible photoisomerization.

The absorption phenotype of the strain overproducing pebA and pebB was almost exclusively due to the incorporation of PEB into the PC α subunit (i.e., CpcA), although a trace of PEB was also found in the AP α subunit (FIG. 9). Whether this was due to the inability of the CpcS/CpcU and CpcT lyases to utilize PEB in vivo, or due to selective degradation of PBP subunits mis-chromophorylated with PEB, was not determined in the studies reported here. Attempts to use the CpcS/CpcU and CpcT lyases to introduce PEB into other PBP subunits in heterologous expression systems have resulted in much lower levels of addition than those seen for CpcEF/CpcA, depending upon the lyase and the phycobiliprotein substrate. This suggests that PBP lyases other than CpcE/CpcF are capable of distinguishing PCB from PEB.

PΦB, the chromophore associated with phytochromes of plants, has a double bond at the 15-16 position and only differs from PCB by the presence of an additional double bond at the C18 position. Because of the structural similarity to the native PCB, it was expected that over-expression of PΦB synthase (HY2) might be much less detrimental to the cell than over-expression of pebA and pebB. During growth in continuous light, expression of HY2 might be required for viability in the HY2-expressing strain in which pcyA has been inactivated. Except for the slower growth rate of this strain at limiting light intensities, the replacement of PCB by PΦB seemed to produce no additional deleterious effects. This is reflected by the fact that the mutant and wild-type strains had nearly identical growth rates when the strains were grown at high light intensity (FIG. 14).

Previous studies on the phycocyanins of red algae and marine Synechococcus sp. strains have shown that at least four alternative PCs occur. The red-alga Porphyridium cruentum synthesizes an R-PC in which the β-155 Cys carries a PEB chromophore but the α-84 and β-84 positions carry PCB chromophores. In Synechococcus sp. WH8103, the β-84 position carries a PCB chromophore, but the α-84 and β-155 positions carry PEB chromophores. The α-84 position of Synechococcus sp. WH8501 PC carries a PUB chromophore, while the two β-subunit cysteines carry PCB chromophores. Finally, there is trichromatic type of R-PC found in Synechococcus sp. WH8102 that contains PUB on α-84, PEB at β-153 and PCB at β-82. The configuration of one PEB on the α phycocyanin subunit and two PCBs on the β phycocyanin subunit is the same configuration reported to be found naturally on R-PC-III of Synechococcus strain WH7805. Phycocyanins as a group exhibit the highest amount of diversity in chromophore content with members being known to bind at least one of each of the 4 possible cyanobacterial chromophores PCB, PEB, PVB, and PUB. The β-84 position invariably possesses PCB however.

These observations, as well as data showing that PE subunits can accommodate PUB chromophores in place of PEB chromophores, demonstrate that at least some PBP subunits can readily accommodate alternative chromophores.

### Example 3

### Use of Exogenously Added Biliverdin to Circumvent the Need for Oxygen for the Formation of the Linear Tetrapyrrole Chromophore of a Recombinant Phycobiliprotein

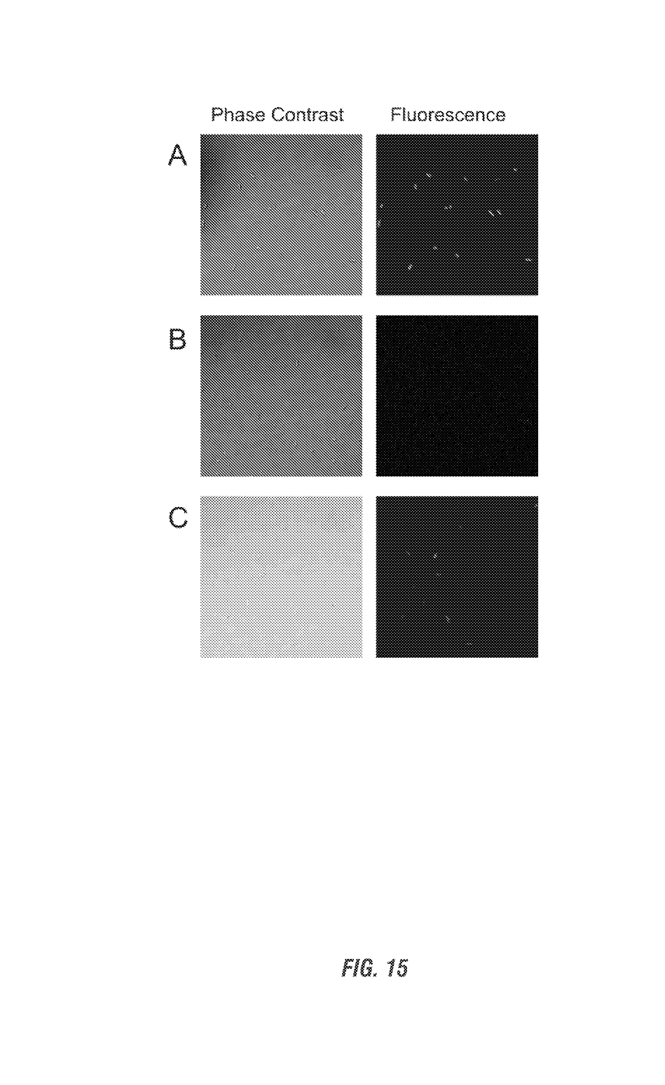

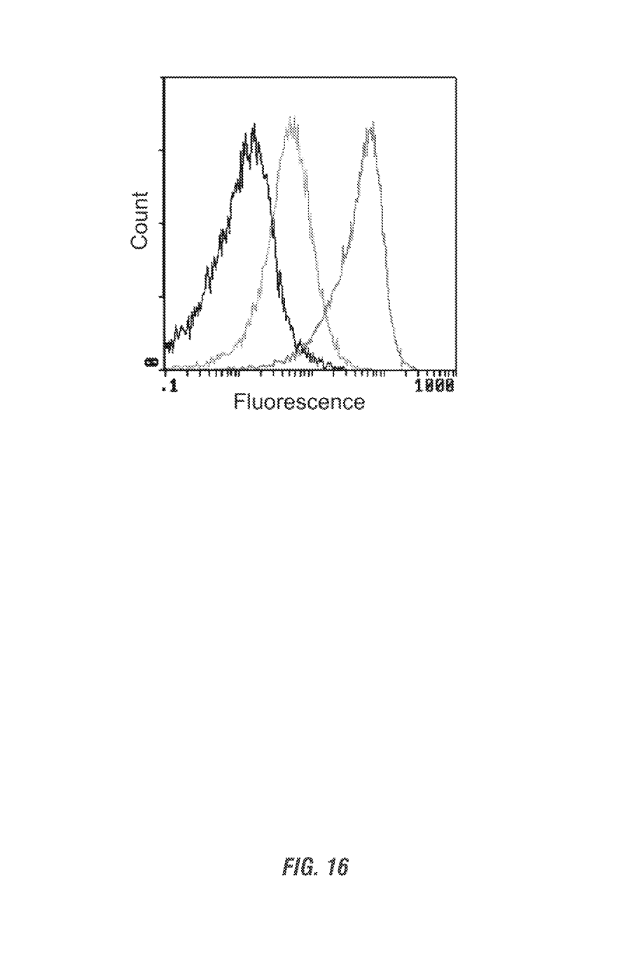

Recombinant Escherichia coli cells co-expressing five genes, hox1, pebS, CpcA, CpcE and CpcF produce the highly fluorescent holo phycobiliprotein subunit HT-CpcA-PEB when grown aerobically and can be readily visualized using fluorescence microscopy (FIG. 15A) or examined using flow cytometry (FIG. 16, red line). However when the same culture is grown anoxically, the cells fail to produce any fluorescent protein and are not able to be detected using fluorescence microscopy (FIG. 15B) and have only background levels of fluorescence when examined using flow cytometry (FIG. 16, black line). This is due to an inability of the cells to perform the first step in the formation of the linear tetrapyrrole chromophore, specifically the opening of the heme ring by the enzyme heme oxygenase which requires oxygen. Commonly used fluorescent proteins such as GFP and its related derivates also require oxygen for the formation of the chromophore and because of this the utility of such fluorescent proteins to study anoxically grown cultures is severely limited. Unlike for GFP however, the requirement for oxygen can be overcome when using fluorescent recombinant phycobiliproteins with the addition of biliverdin to the media of anoxically growing cultures expressing the appropriate genes (FIG. 15C, and FIG. 16, green line). When biliverdin is added to the culture, cells take up the bilin precursor and reduce it using the bilin reductase to make the appropriate chromophore. The fact that cells can readily take up and utilize exogenously added biliverdin also has the added benefit of allowing cells which do not posses a heme oxygenase gene to successfully produce the holo-phycobiliprotein and thus reduces the number of genes needed to be co-expressed at one time. It is also likely that this methodology would work by adding the bilin end product (in this case phycoerythrobilin (PEB)) and would allow for the further reduction of the genes required to just three, one for the apoprotein, CpcA, and two more for the heterodimeric lyase. Furthermore it is likely that this will also be useful for the synthesis of these fluorescent proteins in cell lines such as mammalian cells where free heme may not be readily available to a heterologously expressed heme oxygenase enzyme.

### Example 4

### Characterization of the Activities of the CpeY, CpeZ, and CpeS Bilin Lyases in Phycoerythrin Biosynthesis in Fremyella Diplosiphon Strain Utex 481

When grown in green light, Fremyella diplosiphon strain UTEX 481 produces the red-colored protein phycoerythrin (PE) to maximize photosynthetic light harvesting. PE is composed of two subunits, CpeA and CpeB, which carry two and three phycoerythrobilin (PEB) chromophores, respectively, that are attached to specific Cys residues via thioether linkages. Specific bilin lyases are hypothesized to catalyze each PEB ligation. Using a heterologous, co-expression system in Escherichia coli, the PEB ligation activities of putative lyase subunits CpeY, CpeZ, and CpeS were tested on the CpeA and CpeB subunits from F. diplosiphon. Purified hexa-histidine-tagged-CpeA, obtained by co-expressing cpeA, cpeYZ, and the genes for PEB synthesis, had absorbance and fluorescence emission maxima at 566 nm and 574 nm, respectively. CpeY alone, but not CpeZ, could ligate PEB to CpeA, but the yield of CpeA-PEB was lower than achieved with CpeY and CpeZ together. Studies with site-specific variants of CpeA (C82S and C139S), together with mass spectrometric analysis of trypsin-digested CpeA-PEB, revealed that CpeY/CpeZ attached PEB at Cys⁸² of CpeA. The CpeS bilin lyase ligated PEB at both Cys⁸² and Cys¹³⁹ of CpeA but very inefficiently; the yield of PEB ligated at Cys⁸² was much lower than observed with CpeY or CpeY/CpeZ. However, CpeS efficiently attached PEB to Cys⁸⁰ of CpeB but neither CpeY, CpeZ nor CpeY/CpeZ could ligate PEB to CpeB.

The light-harvesting antennae in cyanobacteria and red algae are supramolecular complexes, phycobilisomes (PBS), composed of water-soluble and brilliantly colored phycobiliproteins (PBPs) and linker polypeptides. The covalent attachment of phycobilin chromophores to specific Cys residues, usually by enzymes called bilin lyases, results in highly fluorescent holo-PBPs. The attached phycobilin chromophores transfer excitation energy with high quantum efficiency to photosynthetic reaction centers.

The major PBPs, each consisting of α and β subunits, in cyanobacteria are the aqua-colored allophycocyanin (AP) (λₘₐₓ˜650 nm), the blue-colored phycocyanin (PC) (λₘₐₓ˜620 nm), and the red-colored phycoerythrin (PE) (λₘₐₓ˜560 nm). The spectroscopic properties of these proteins are determined primarily by their bilin chromophore(s), which are attached to specific Cys residues. The bilin lyases responsible for the phycocyanobilin (PCB) ligation at the binding sites for all PBPs in Synechococcus sp. strain PCC 7002 have been characterized. Thus far, four types of bilin lyases are known, and each has different characteristics and amino acid sequences. The first bilin lyases to be discovered belonged to the E/F family and are typified by the CpcE and CpcF proteins; these proteins form heterodimeric enzymes that can attach PCB to Cys⁸² of CpcA (α-PC subunit) and can also remove bilins from holo-subunits or transfer bilins from a holo-subunit to an apo-subunit. Recent studies in both cyanobacteria and Escherichia coli have demonstrated that the CpcE/CpcF lyase can attach non-cognate bilins (e.g., phycoerythrobilin (PEB) or phytochromobilin) to CpcA. Paralogs of the cpcE and cpcF genes, usually encoded in operons with phycobiliprotein subunit genes, are known. Some of these paralogs, such as pecE/pecF and rpcG, have been shown to be involved in the attachment and isomerization of bilin chromophores to Cys residues, which usually occur on the α-subunit of some PBPs (e.g., PecA or RpcA). Similar to CpcE/CpcF, these lyases appear to be capable of attaching non-cognate bilins to non-cognate PBP subunits.

The second family of bilin lyases belongs to the S/U family and are typified by CpeS- and CpcS-like proteins, which can be active as monomers, homodimers or heterodimers (CpcS/CpcU). Members of the S/U lyase family do not appear to catalyze the transfer or removal of bilins from holo-PBP subunits, but their protein substrate specificity seems to be broader than that of other lyase types. They can typically recognize many different PBP substrates and attach bilins at their Cys⁸²-equivalent positions. The third family of lyases is called the T-type and is typified by CpcT, an enzyme that attaches PCB at Cys¹⁵³ of CpcB. Lastly, there is a family of PBPs that autocatalytically ligate bilin chromophores. Currently, the sole representative of this family is ApcE. It has been reported that an allophycocyanin subunit (ApcA) is able to self-ligate its bilin chromophore, but phenotypic analyses of bilin lyase mutants in cyanobacteria strongly suggest that bilin lyases such as CpcSU are required for this activity in vivo. It should also be noted that phytochrome and other related photoreceptors also add their chromophores autocatalytically.

PEs are a diverse family of PBPs with extensive variation in subunit composition and spectral properties. Insertions have occurred in the primary amino acid sequences of PEs that have created more chromophore binding sites than occur in other PBP subunits. In the PE of the freshwater cyanobacterium F. diplosiphon, five phycoerythrobilin (PEB) chromophores are ligated to Cys residues at α⁸², α¹³⁹, β⁸⁰, β¹⁶⁵ and β⁴⁸/β⁵⁹ (doubly-linked at rings A and D). The PEs of marine cyanobacteria are even more complex and diverse; they have additional bilin attachment sites that can carry either PEB or phycourobilin (PUB).

Previous studies have suggested that bilin lyases are also required for bilin attachment to PEs. Kahn et al. found that a transposon insertion into the cpeY gene resulted in diminished levels of PE in F. diplosiphon cells grown in green light. The cpeY and cpeZ genes, encoded in the PE operon cpeBAYZ, are paralogs of the cpcE and cpcF lyase genes. Kahn et al. suggested that CpeY and CpeZ function in PE biosynthesis, possibly as a lyase in the attachment of PEB to the α or β subunits Zhao et al. later showed that the CpcS-type lyase from Nostoc sp. PCC 7120 (an organism that does not contain PE) was capable of attaching PEB to Cys⁸² on CpeA and CpeB from F. diplosiphon; this result suggested that this type of lyase had broad substrate specificity and might recognize Cys⁸² on any type of PBP subunit (including CpcB, ApcA, ApcB, ApcD, ApcF, CpeA, and CpeB) with the exception of CpcA, RpcA, and PecA.

In this study, we have used a biochemical approach with recombinant enzymes to characterize the roles of CpeY, CpeZ and CpeS in PEB addition to PE subunits in F. diplosiphon strain UTEX 481. CpeY alone could ligate PEB to apo-CpeA, but the yield was lower (˜60%) than when both CpeY and CpeZ were present. Site-directed mutagenesis of cysteine residues on CpeA and mass spectrometry showed that CpeY alone and CpeY/CpeZ ligate PEB to Cys⁸² of CpeA. A very small amount of PEB was found attached to Cys¹³⁹ in this sample as well, but CpeY/CpeZ was unable to attach PEB to the CpeA (C82S) mutant. CpeS was capable of PEB attachment to Cys⁸⁰ of CpeB as well as to both Cys⁸² and Cys¹³⁹ of CpeA; however, lower amounts of PEB were ligated to the apo-CpeA than obtained with CpeY/CpeZ. This result strongly suggested that the main function of CpeS is to attach PEB to Cys⁸⁰ of CpeB.

### Experimental Procedures

### Construction of Expression Vectors

Plasmids used in this study are listed in Table 3. Some of the expression vectors used in this study were previously described. All expression constructs newly produced for this study were sequenced at the W. M. Keck Conservation and Molecular Genetics Laboratory (University of New Orleans) to confirm that no mutations had been introduced during PCR amplification and cloning.

Each gene was amplified by PCR from F. diplosiphon chromosomal DNA using primers listed in Supplemental Table 4, and each resulting amplicon was cloned into a Duet vector as listed in Supplemental Table 3 (Novagen, Madison, Wis.) after digestion with restriction enzymes (engineered into the primers; underlined in the sequences in Supplemental Table 4). As indicated in Supplemental Table 3, a hexa-histidine tag was engineered into the constructs for producing CpeA, CpeB and CpeZ. The plasmid pPebS was a generous gift from Dr. Nicole Frankenberg-Dinkel; it contains the ho1 (heme oxygenase) and pebS (PEB synthase) genes from a myovirus that infects Prochlorococcus spp. E. coli strains harboring this plasmid produce PEB from heme.

Site-Directed Mutagenesis of cpeA and cpe

Plasmid pCpeA was used as a template for generating mutations in cpeA. The Transformer™ Site-Directed Mutagenesis Kit from Clontech Laboratories, Inc. was used to create mutated genes for the production of three CpeA variants: CpeA (C82S), CpeA (C139S) and CpeA (C82S, C139S). The primers used were CpeA (C82S), CpeA(C139S), and pETDuet (XhoI del) (Supplemental Table 1). Plasmid pCpeB (Supplemental Table 1) was used as a template for generating mutations in cpeB by the same method. Three variants of CpeB were produced: CpeB (C805), CpeB (C165S) and CpeB (C48S/C59S). The primers used were CpeB (C805), CpeB (C165S), CpeB (C48S, C59S), and pETDuet-1 (XhoI del;)

### Heterologous Expression and Purification of Recombinant Proteins

Expression plasmids were co-transformed into E. coli BL21 DE3 cells as required, and colonies were selected on Luria-Bertani (LB) plates in the presence of the appropriate combination of antibiotics (Table 4) at the following concentrations: ampicillin (Ap: 100 μg ml⁻¹), chloramphenicol (Cm: 34 μg ml⁻¹), kanamycin (Km: 50 μg ml⁻¹), spectinomycin (Sp: 100 μg ml⁻¹). To produce PEB using the pPebS expression plasmid, a 50-ml overnight starter culture was added to 1 L of LB medium with the appropriate combination of antibiotics. This culture was shaken at 37° C. for 4 h until the optical density reached OD_{600 nm}=0.6. Production of T7 RNA polymerase was induced by the addition of 1 mM isopropyl β-D thiogalactoside (IPTG). Cells were incubated with shaking at 190 rpm at 18° C. for another 16 h before they were harvested by centrifugation at 10,000×g for 10 min. Cell pellets were stored at −20° C. until required.

E. coli cells containing recombinant proteins were thawed and resuspended in Buffer 0 (20 mM Tris-HCl, 100 mM NaCl, pH 8.0) at 2.5 ml g⁻¹ (wet weight) along with protease inhibitor cocktail tablets (“Complete Mini” from Roche, Mannheim, Germany). The cells were lysed, and the hexa-histidine-tagged recombinant proteins were purified as previously described. The recombinant protein(s) were exhaustively dialyzed with buffer O containing 10 mM 2-mercaptoethanol overnight at 4° C. to remove the imidazole introduced during elution.

### Fluorescence Emission and Absorbance Spectra

Fluorescence emission spectra were recorded with a Perkin Elmer LS55 fluorescence spectrophotometer (Waltham, Mass.) with slit widths set at 10 nm (excitation and emission). For recombinant PBPs, the excitation wavelength was set at 490 nm and samples were diluted to achieve a standard absorbance level ˜0.05 OD (at λₘₐₓ) prior to recording the fluorescence emission spectra. Negative control samples (e.g., no lyase addition), which had little or no attached chromophore, were not diluted, as their OD values were generally less than 0.05. Absorbance spectra were acquired using a lambda 35, dual-beam UV-Vis spectrophotometer (Perkin Elmer, Waltham, Mass.). In order to compare the amount of relative fluorescent CpeA produced in the presence of the CpeY/CpeZ, CpeY, or CpeS lyases, these proteins were purified from the same volume of E. coli cultures (the pellets obtained were within 5% of each other in wet weight). The CpeA protein concentration was also estimated for each of these on SDS-polyacrylamide gels. The relative fluorescence intensity was multiplied by the dilution factor used, and then this was divided by the CpeA concentration to estimate the proportion of CpeA produced that was fluorescent. The value obtained for CpeY/CpeZ was set to 100%, and the other values were scaled accordingly (Table 3).

### Protein and Bilin Analysis

Polypeptides were resolved by polyacrylamide gel electrophoresis (PAGE; 15% w/v) in the presence of sodium dodecyl sulfate (SDS), and visualized by staining with Coomassie Blue as described. To detect PEB linked to proteins, gels were soaked in 100 mM ZnSO₄ for approximately 5 min and the zinc-enhanced fluorescence, indicative of bilin attachment, was visualized using an FX imaging system (BioRad, Hercules, Calif.) with excitation at 532 nm.

### Calculating Fluorescence Quantum Yield

The fluorescence quantum yield relative to cresyl violet (in ethanol Φ_{f}=0.59; Sigma Aldrich) of CpeA-PEB or CpeB-PEB was calculated as described using a Perkin Elmer LS55 fluorescence spectrophotometer (Waltham, Mass.) with slit widths set at 10 nm (excitation and emission). The fluorescence emission spectrum was acquired from 570 to 800 nm. The fluorescence quantum yield of the sample was calculated in comparison with the standard using the following equation:

A₁ε₁/A₂ε₂=Φ_{f1}/Φ_{f2}

where A is the absorbance value at the maximum, ε is the area of the fluorescence emission spectrum from 570 nm to 800 nm, and Φ_{f} is the fluorescence quantum yield.

### Protein-Protein Interaction Assays

Pull-down assays between HT-CpeZ and CpeY were performed using whole-cell extracts from a CpeY expression culture (pCpeY) and purified HT-CpeZ (from a pCpeZ culture).

### Immunoblotting Analysis

Antibodies against recombinant F. diplosiphon CpeA-PEB (produced with CpeY/CpeZ) and CpeB-PEB (produced with CpeS) were generated in rabbits (YenZym Antibodies, South San Francisco, Calif.). Immunoblotting analysis was performed as described in using antisera at a 1:5000 dilution.

### Tryptic Digestion of Proteins

Purified CpeA-PEB or CpeB-PEB was dialyzed against 2 mM sodium phosphate buffer, pH 7.0, 1 mM 2-mercaptoethanol, concentrated by ultrafiltration through an Amicon YM10 (Millipore, Billerica, Mass.), and subjected to digestion with trypsin following the protocol. The reaction was quenched by adding 30% (v/v) glacial acetic acid, followed by passage through a C-18 Sep-Pak (Waters Corp., Milford, Mass.) cartridge. The eluted sample was vacuum dried and stored at −20° C. for HPLC analysis.

### High Performance Liquid Chromatography

Tryptic peptides were separated on a C₁₈ reverse-phase HPLC column (5 μm×10 mm×250 mm; Waters Corp., Milford, Mass.) using a Waters HPLC equipped with a 600E pump and a photodiode array detector. The peptide separation was carried out as described by Arciero et al., using 0.1 M Na-phosphate, pH 2.1 as (Solvent A) and acetonitrile (Solvent B). The bilin peptides were eluted with an increasing concentration of acetonitrile (35% to 100%) and were monitored at 560 nm. The eluted samples were vacuum-dried and kept at −20° C. for mass spectrometric analysis.

### Mass Spectrometry

MALDI MS and tandem MALDI MS/MS experiments were performed on an Applied Biosystems (Foster City, Calif.)/MDS Sciex (Concord, Ontario) 4800 MALDI TOF/TOF spectrometer. Mass spectral acquisitions were obtained in the reflectron mode using a Nd:YAG laser operated at 355 nm. The matrix used was α-cyanohydroxycinnamic acid (Sigma Aldrich) at 15 mg mL⁻¹ in 50%, (v/v) acetonitrile (Sigma Aldrich)/0.1% (v/v) trifluoroacetic acid (Sigma Aldrich). An aliquot (2 μL) from each fraction was mixed with matrix (2 μL); the mixture was homogenized, and an aliquot (0.75 μL) was spotted on a MALDI 384-well plate and air-dried prior to analysis.

### Results

Characterization of Bilin Lyase Activity of CpeY and CpeZ with CpeA

The cpeY and cpeZ genes occur downstream of the cpeBA genes, which encode the β and α subunits of PE, respectively. Based upon their sequence similarity, CpeY and CpeZ belong to the CpcE/CpcF family of bilin lyases. CpeY and CpcE (from Synechocystis strain PCC 6803) are 22% identical and 32% similar. CpeZ and CpcE are 25% identical and 38% similar, while CpeZ and CpcF (from Synechocystis strain PCC 6803) are 23% identical and 37% similar. Transposon mutants and complementation studies in F. diplosiphon suggested that these two proteins are involved in PE biogenesis, but their specific roles were not elucidated. An in vivo E. coli heterologous co-expression system was used to test whether either of these genes encodes a bilin lyase. Constructs used in this study are listed in Supplemental Table 5.

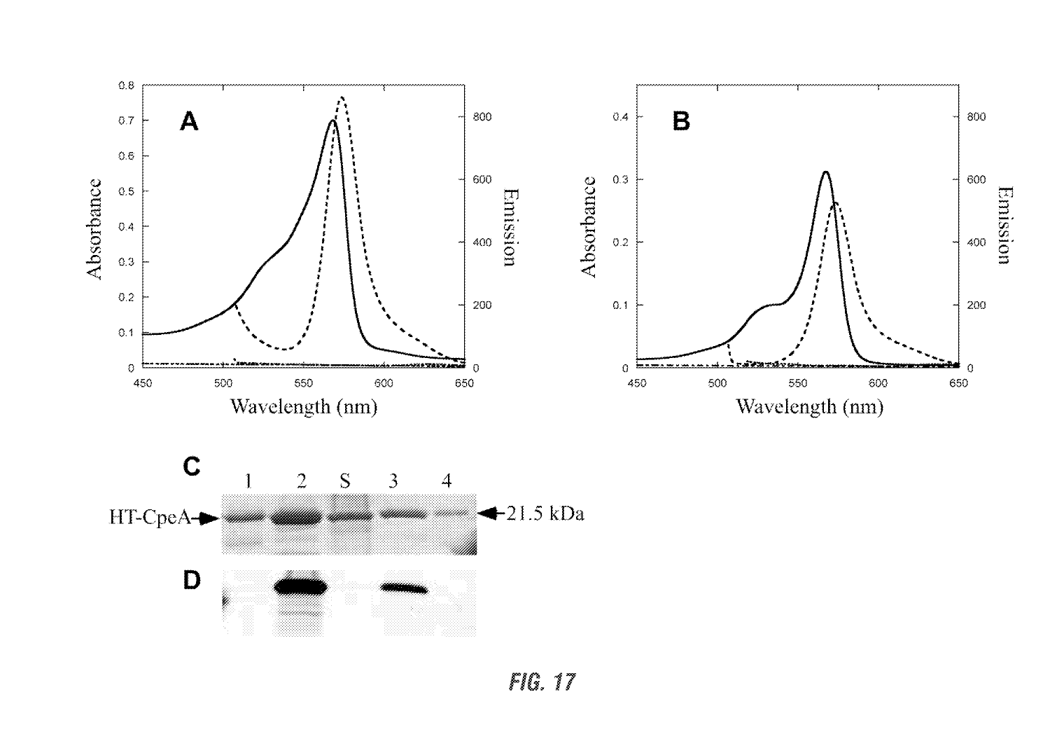

E. coli cells containing plasmids pCpeA and pPebS (i.e., no lyase present) had no significant color after induction with IPTG (data not shown), but cells containing these two plasmids in addition to pCpeYZ were bright pinkish-red. CpeA-PEB purified from these cells had an absorbance maximum at 560 nm (FIG. 17A) and was intensely fluorescent with an emission maximum at 574 nm (FIG. 17A), whereas the purified protein obtained from the cells containing only pCpeA and pPebS did not have any significant absorbance or fluorescence emission (FIG. 17A). CpeZ and CpeY were also tested individually to determine if either protein alone could attach PEB to CpeA. The fluorescence emission amplitude for CpeA-PEB purified from cells containing pCpeA, pPebS and pCpeY showed that CpeY had significant activity by itself, but the amount of fluorescent product was lower than when both CpeY and CpeZ were present (FIG. 17B). The relative yields of CpeA-PEB fluorescent product purified after co-expression with PebS along with either CpeY or CpeZ are given in Table 3. When CpeA was co-expressed with CpeY, CpeA-PEB product was highly fluorescent (FIG. 17B, dashed lines); however, the CpeA product from co-expression with the other subunit, CpeZ, was not fluorescent (FIG. 17B, dashed dotted line, Table 3). The three CpeA samples purified from E. coli cells were analyzed by SDS-PAGE (FIG. 17C). Bilin addition to CpeA was detected by zinc staining of the gel to enhance bilin fluorescence (FIG. 17D); subsequent staining of the same gel with Coomassie Blue revealed the protein content (FIG. 17C). The CpeA purified from cells expressing both CpeY and CpeZ was highly fluorescent after Zn-staining (FIG. 17D; lane 2), but CpeA purified from cells containing no lyase subunit or with CpeZ alone was not fluorescent after Zn-staining. Thus, little or no ligation of PEB occurred in the absence of the CpeY subunit. (FIG. 17D, lanes 1 and 4, respectively). However, CpeA purified from cells coexpressing CpeY produced a fluorescent product with a yield that was ˜60% of that achieved in the presence of both CpeY and CpeZ (Table 1); this observation suggested that CpeZ enhances the PEB ligation activity of CpeY. When CpeA was coexpressed with CpeY or CpeY/CpeZ and the genes for PEB biosynthesis, it accumulated in a soluble form (FIG. 17C, lanes 2 and 3). However, immunoblotting analyses with antibodies to CpeA showed that CpeA formed inclusion bodies when expressed in the absence of lyase subunits in E. coli.

Because the presence of CpeZ enhanced the bilin ligation activity of CpeY, and because other bilin lyases such as CpcE and CpcF have been shown to form heterodimers, we tested whether CpeY and HT-CpeZ interact. The CpeY and HT-CpeZ proteins coproduced in E. coli were soluble, but CpeY did not copurify with HT-CpeZ (Supplemental FIG. 3). No copurification of CpeY was observed when it was incubated together with both CpeA-PEB and HT-CpeZ.

### Analysis of the Cysteine Residues on CpeA Chromophorylated by the CpeY/CpeZ Lyase

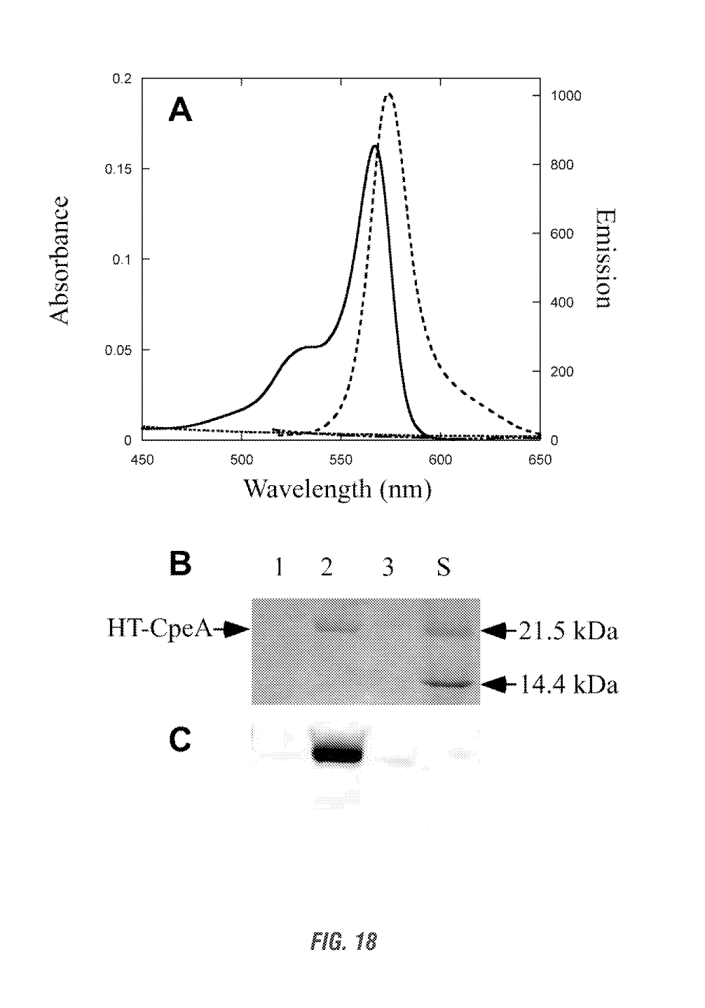

The holo-CpeA (α-PE subunit) isolated from F. diplosiphon carries PEB chromophores at Cys⁸² and Cys¹³⁹. To test the site specificity of the CpeY/CpeZ bilin lyase, site-specific variants of CpeA (C82S, C139S and C82S/C139S) were produced in which cysteine residues were changed to serine. Each mutant gene was co-expressed with the CpeY/CpeZ lyase and the enzymes to synthesize PEB, and the CpeA produced was purified. The results of the absorbance and fluorescence emission measurements on these proteins are shown in FIG. 18 and Table 1. Only the C139S CpeA variant was a substrate for PEB ligation by CpeY/CpeZ, and the product had an absorption maximum at 566 nm and a fluorescence emission maximum at 574 nm (FIG. 18A). These values were identical to those for CpeA-PEB described above, and these results indicate that Cys⁸² is the residue that is chromophorylated with PEB by the CpeY/CpeZ lyase. The purified C82S and C82S/C139S variants of CpeA produced in the presence of the CpeY/CpeZ lyase and PEB synthesis enzymes had no significant fluorescence emission (FIG. 18A and Table 1). Similarly, no fluorescent products were observed when any of the variant proteins were produced in the absence of the lyase subunits (data not shown). The CpeA variants produced in these experiments were also analyzed by SDS-PAGE (FIGS. 18B and 18C). Bilin addition to each protein was examined by zinc-enhanced fluorescence of the gel (FIG. 18C). The purified C139S CpeA variant was fluorescent due to the presence of covalently attached PEB (FIG. 18C, lane 2). After staining the same gel shown in FIG. 2C with Coomassie Blue (FIG. 18 B), it was apparent that CpeA only accumulated in the soluble fraction when PEB had been ligated to the protein (lane 2, FIG. 18B). As verified by immunoblot analyses using anti-CpeA antibodies, the non-chromophorylated CpeA variant proteins produced in these cells accumulated in an insoluble form in inclusion bodies, (data not shown, but results similar to those shown in Supplemental FIG. 18A). From these experiments, we concluded that the CpeY/CpeZ bilin lyase specifically attaches PEB to Cys⁸² of CpeA. We will refer to this protein as CpeA-PEB to differentiate it from a true holo-CpeA carrying PEB at both Cys⁸² and Cys¹³⁹.

### Mass Spectrometry of Tryptic Peptides

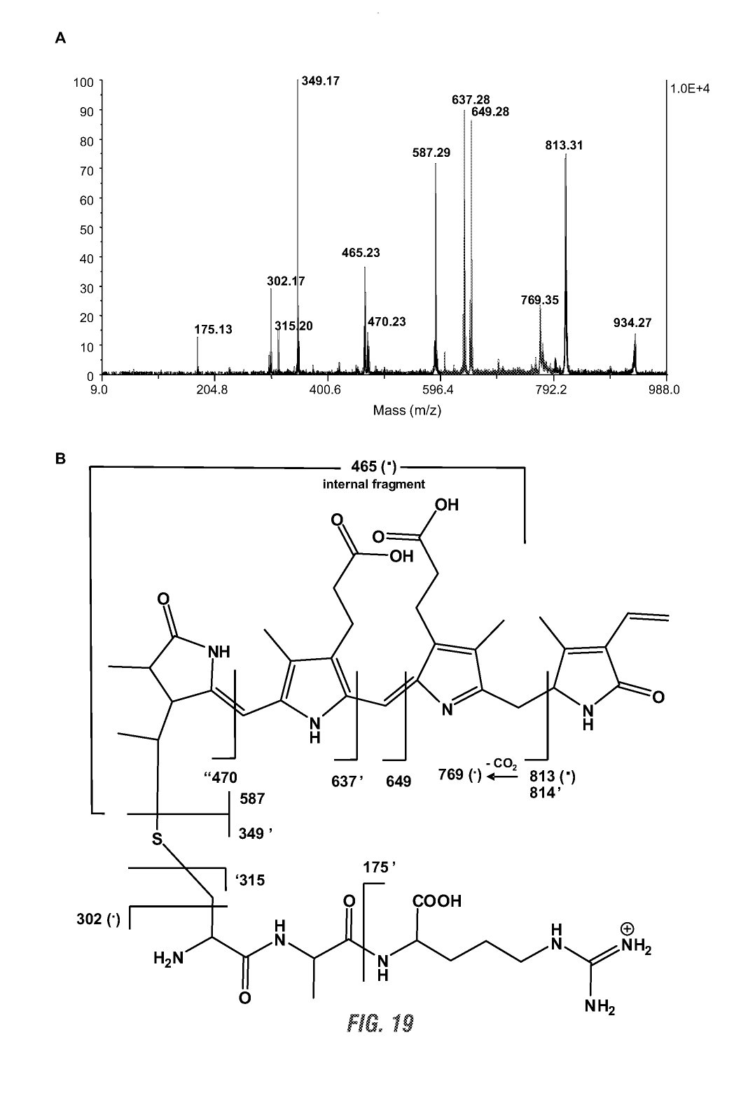

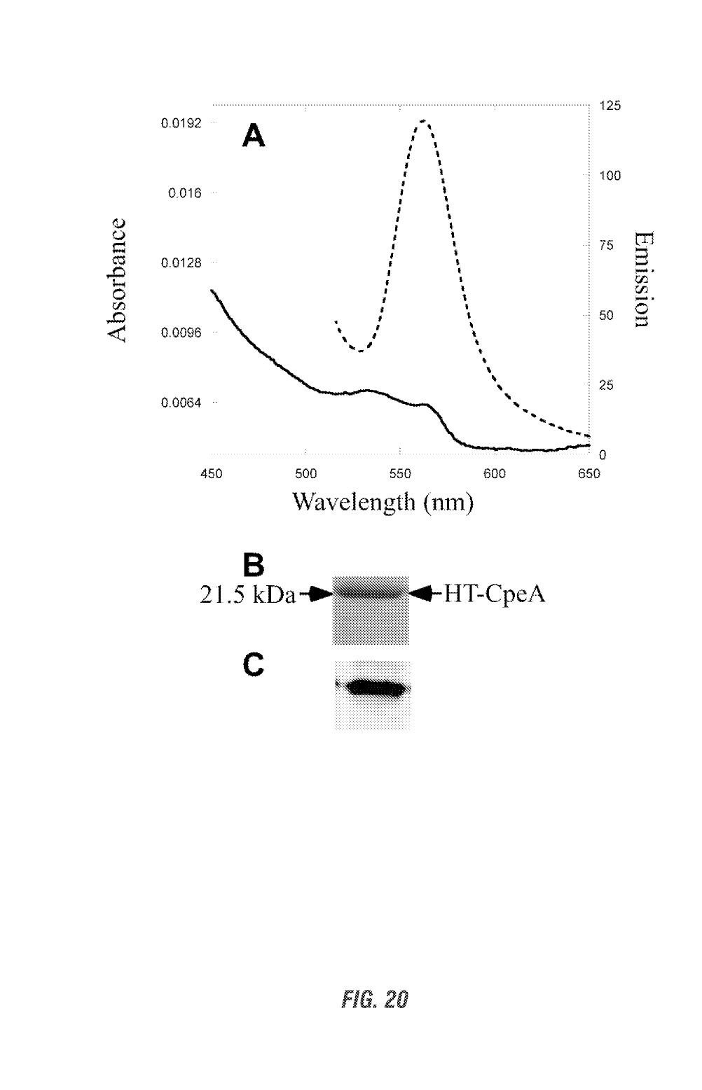

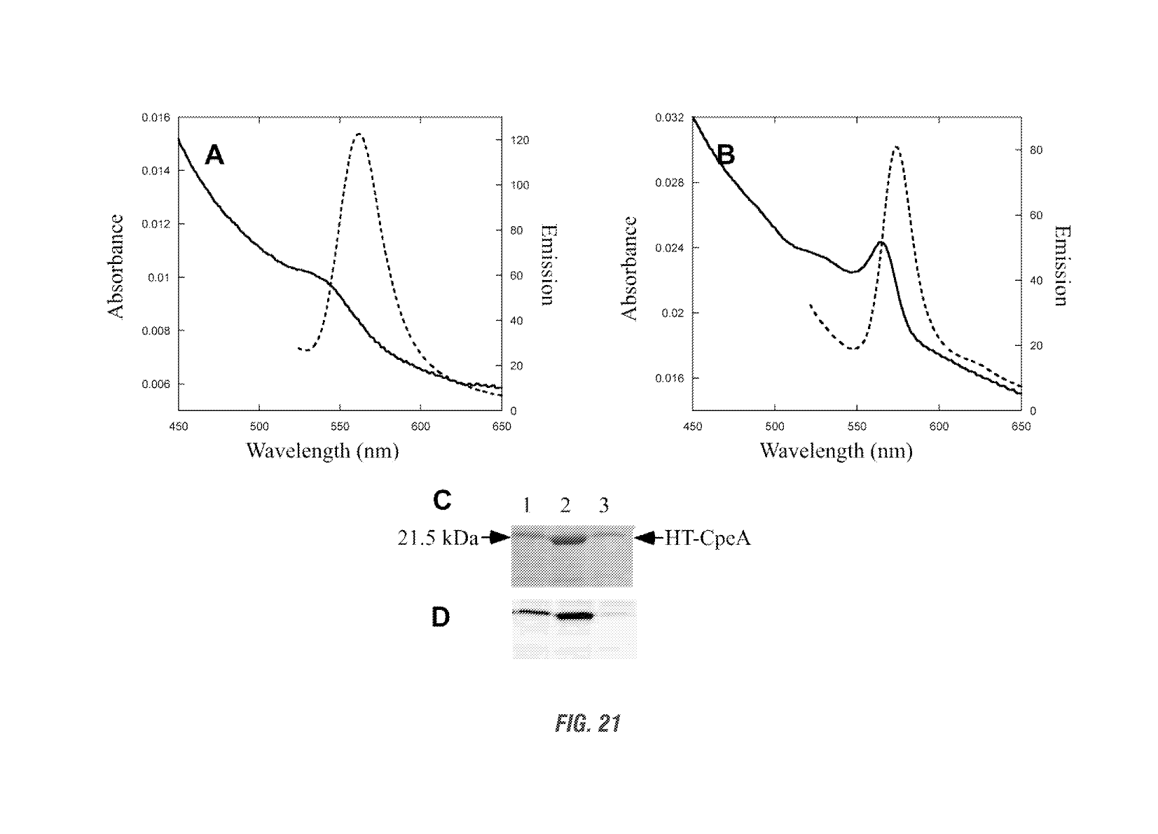

Because non-chromophorylated CpeA and its variants exhibited limited solubility when expressed in E. coli, it seemed plausible that a bound bilin at this central position within CpeA (at Cys⁸²) increased the solubility and stability of CpeA in E. coli cells. Even though our site-specific variant experiments showed that CpeY/CpeZ ligates PEB at Cys⁸², this did not rule out the possibility that CpeY/CpeZ also ligates PEB at Cys¹³⁹ but only after it attaches PEB at Cys⁸². Therefore, we analyzed the CpeA (non-variant) produced in the presence of pCpeA, pCpeYZ, and pPebS to determine if PEB was attached to more than one Cys on CpeA by subjecting the recombinant protein to tryptic digestion followed by mass spectrometry. The digested peptides were separated on a C₁₈ reversed-phase HPLC (RP-HPLC) column. Two peaks were observed at 550 nm (specific for PEB); one major peak at 23.5 min and one minor peak at 23.0 min were collected (See Supplemental FIG. 20). Each fraction obtained from HPLC separation was subjected to MALDI MS and tandem MS analysis to identify unambiguously the location of the covalently attached PEB on CpeA. Tandem mass spectrometry using MALDI MS of peptides resulting from the tryptic digestion of the covalent complex CpeA-PEB was performed to accomplish this. We sought to identify one or more peptides produced upon digestion that contained ligated PEB (FIG. 19). A peak at m/z 935 appeared in both fractions. FIG. 19A shows the MS/MS spectrum of the precursor at m/z 935. In particular, there are two main peaks of interest at m/z 587 and 349. The peak at m/z 587 is attributed to protonated PEB. The peak at m/z 349 matches a tripeptide, which has the sequence (K) CAR (D) and contains Cys⁸². The remaining major peaks were also assignable in a manner consistent with covalent attachment of PEB to Cys⁸². The scheme in FIG. 19B summarizes the structures of the assigned peaks.

The above spectral interpretation suggests that, by applying sufficient collision energy in tandem mass spectrometry experiments, it was possible to break the thioether bond and separately detect the chromophore (m/z 587) and the peptide (m/z 349). The structure of the chromophore, which is highly conjugated, favored the formation of product ions that were stabilized by resonance. The large number of peaks enabled a thorough structural elucidation of the peptide-PEB covalent complex.

In a follow-up analysis, we specifically investigated whether PEB attachment occurred at Cys¹³⁹. We were able to unambiguously identify a peptide at m/z=503 that contained the unmodified cysteine at position 139 (sequence: (R) GCAPR (D)). If attachment of PEB occurred to Cys¹³⁹, then it should have been possible to detect a chromopeptide with m/z=1089 (503+586). An ion with this m/z ratio was indeed detected in one fraction. The product ion mass spectrum from this m/z 1089 precursor yielded a minor peak at m/z 587 and a peak at m/z 503 corresponding to the neutral loss of 586. This m/z 503 peak thus represents (R) GC¹³⁹ APR (D) that has lost the bilin moiety. Furthermore, a comparison of the peak heights observed in MALDI mass spectra that simultaneously contained m/z 1089 and 935 revealed that the former peak was quite minor compared to the latter. Although peak heights observed in MALDI mass spectra are not strictly reliable for quantification, these data strongly suggested CpeY/CpeZ predominantly attached PEB to Cys⁸².

### Does CpeS Also Chromophorylate CpeA?

Using a recombinant E. coli in vivo assay system, Zhao et al. reported that Nostoc sp. PCC 7120 CpcS (formerly denoted CpeS1) is a “near universal lyase” that adds bilins to Cys⁸² on most biliproteins, including the non-cognate substrate, CpeA, from F. diplosiphon. (Note that Nostoc sp. PCC 7120 does not synthesize PE, so this bilin lyase should be designated as CpcS1 not as CpeS1). Having demonstrated that the CpeY/CpeZ lyase attaches PEB primarily to Cys⁸² of CpeA, it was important to compare the activities of the CpeY/CpeZ and CpeS lyases on the substrate CpeA in E. coli. The cognate cpeS gene from F. diplosiphon was cloned to create plasmid pCpeS (see Supplemental Table 1). FIG. 20A shows the absorbance and fluorescence emission spectra of CpeA purified from cells co-producing CpeA, CpeS, and enzymes for PEB synthesis. The yield of CpeA-PEB was much lower Table 1) when the coproduced lyase was CpeS rather than CpeY/CpeZ. However, the absorbance and fluorescence properties of the resulting CpeA proteins were similar (Table 1). The CpeA-PEB produced in the presence of CpeS was analyzed by SDS-PAGE. The Zn-enhanced fluorescence in FIG. 20C shows that PEB is covalently attached to the CpeA protein, but the amount of fluorescent CpeA-PEB produced by CpeS in 1-liter culture was estimated to be less than 1% of that obtained in the presence of CpeY/CpeZ (Table 1).

The site-specific variants of CpeA were additionally used to investigate the activity of CpeS, and these results are shown in FIG. 21 and Table 1. Interestingly, small amounts of fluorescent product were obtained for both CpeA (C82S) and CpeA (C139S), but no significant fluorescence emission was observed when CpeA variants were co-expressed in absence of the CpeS lyase. These results suggested that CpeS could ligate PEB to both cysteines on CpeA but not very efficiently. The fluorescence emission maxima for the two variants were different; this suggested that PEB was bound in different protein environments within the two variants, thereby affecting its absorbance and emission properties (Table 3). In further support of this interpretation, two PEB-containing peptides were observed after tryptic digestion of CpeA-PEB chromophorylated in the presence CpeS (data not shown). Although CpeS can attach PEB to both Cys residues on CpeA, the low activity level suggested that CpeS is unlikely to be the cognate PEB lyase for either of these positions.

Comparison of the PEB Ligation Activity of CpeY/CpeZ and CpeS Bilin Lyases with CpeB

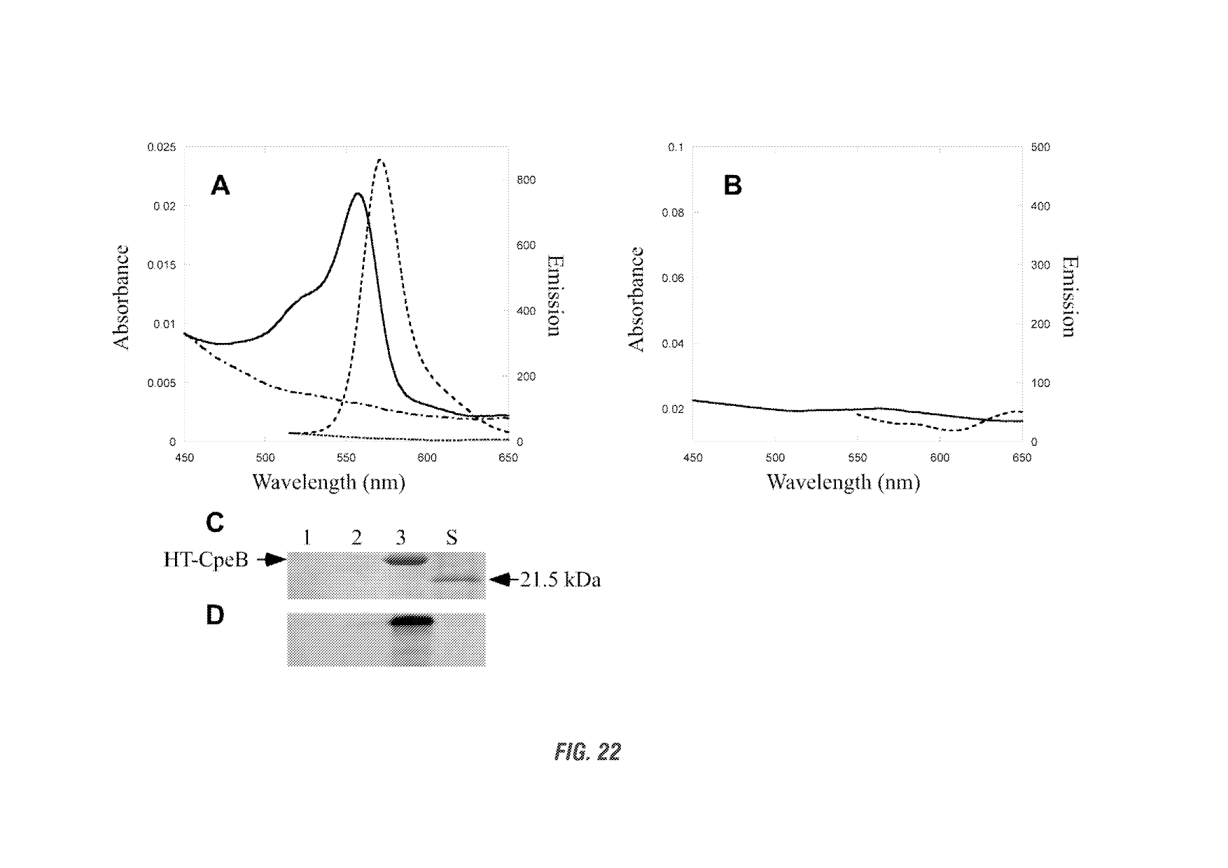

Because CpeS is unlikely to be a cognate lyase for CpeA, we tested CpeS activity with CpeB as a substrate and investigated whether CpeY/CpeZ could also ligate PEB to CpeB. Holo-CpeB (β-PE) synthesized in F. diplosiphon has three PEB chromophores attached to four Cys residues: Cys⁸⁰ and Cys¹⁶⁵ carry singly linked PEBs and Cys⁴⁸ and Cys⁵⁹ carry a doubly linked PEB (at C3¹ and C18¹ of the bilin). Two different stereoisomers of PEB occur in CpeB; the R-isomer is present at C3¹ on Cys⁸⁰ and Cys⁴⁸/Cys⁵⁹, and the S-isomer is present at C3¹ on Cys¹⁶⁵. Using the in vivo co-expression system, CpeB was coproduced with enzymes for PEB synthesis and either no lyase, CpeS, CpeY, CpeZ, or CpeY/CpeZ (Supplemental Table 1). FIG. 22A shows the absorbance and fluorescence emission spectra of the resulting CpeB product after purification from cells producing no lyase or CpeS. The CpeB-PEB produced in the presence of CpeS had an absorbance maximum at 560 nm and a fluorescence emission maximum at 571 nm. No significant ligation of PEB to CpeB occurred in the absence of CpeS (FIG. 22A, dashed dotted line). When CpeB was purified from cells expressing CpeY and CpeZ in addition to the PEB synthesis enzymes, no significant absorbance or fluorescence emission of the product was observed as shown in FIG. 22B. The same result was observed when only CpeY or CpeZ was present as the bilin lyase subunit (data not shown). These data strongly suggested that CpeB is not a substrate for the CpeY/CpeZ lyase.

After separating the proteins on SDS-PAGE (FIGS. 22C and 22D), the bilin content of each protein was examined by Zn-enhanced fluorescence staining of the SDS-polyacrylamide gel (FIG. 22D), and proteins were detected after staining the same gel with Coomassie Blue (FIG. 22C). Very little apo-CpeB could be purified from the control reactions (no lyase present, lane 1 in FIG. 22C and FIG. 22D) or under conditions for which no bilin was attached by CpeY/CpeZ (lane 2 in FIG. 22C and FIG. 22D). CpeB only accumulated in soluble form in E. coli after PEB was covalently attached in the presence of CpeS (FIGS. 22 C and 22D, lane 3). Immunoblotting analyses showed that CpeB was mainly in inclusion bodies unless CpeS was also co-expressed.

### Analysis of Specific Cys Residue(s) of CpeB Chromophorylated by CpeS

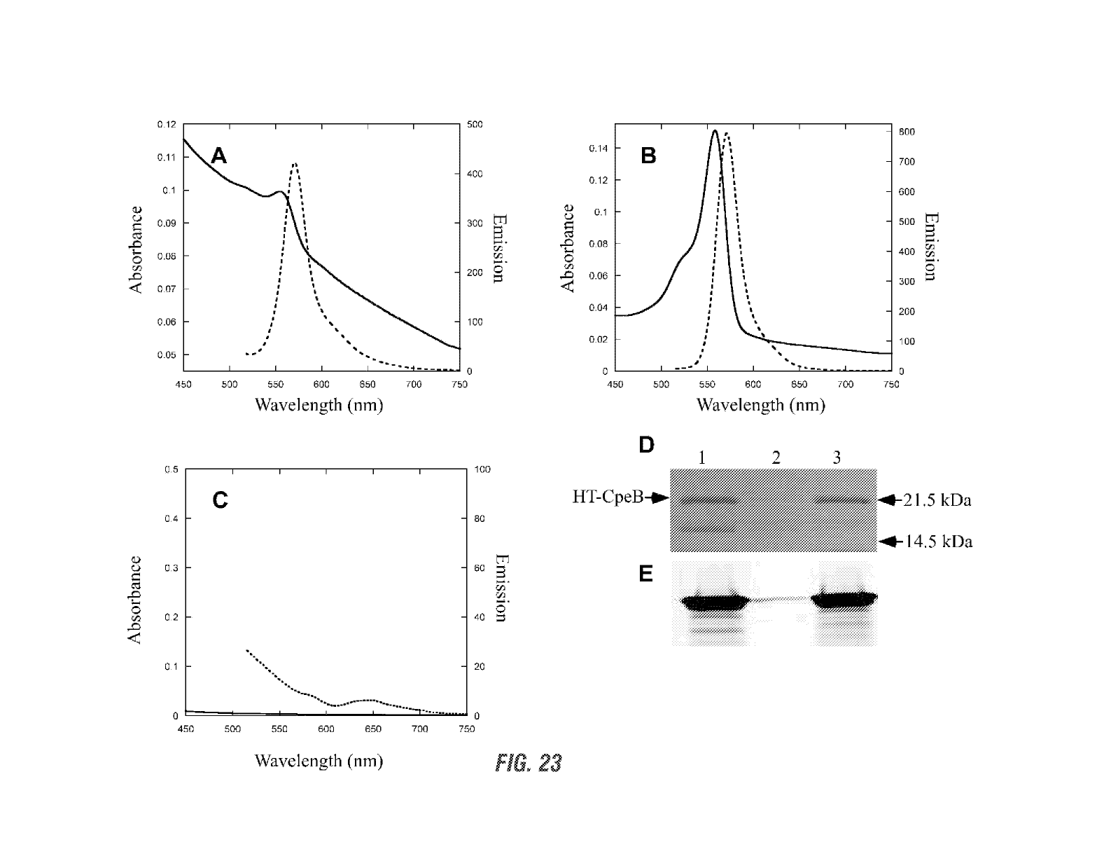

To determine the site specificity of the CpeS bilin lyase, site-specific variants of CpeB (C805, C165S and C48S/C59S) were produced as substrates for CpeS. After coproduction of each site-specific variant with CpeS and the enzymes for PEB synthesis, the CpeB product was purified, and the results of absorbance and fluorescence emission measurements are shown in FIG. 23. The C165S and C48S/C59S CpeB variants had absorbance maxima at 560 nm and fluorescence emission maxima at 571 nm (FIGS. 23A and 23B, respectively), but the CpeB (C805) variant had no significant absorbance or fluorescence emission (FIG. 23C). Control experiments without the CpeS lyase with all CpeB variants were also performed, and in all cases no fluorescent product was observed. The CpeB produced in these experiments was analyzed by SDS-PAGE. Bilin addition to each protein was examined by Zn-enhanced fluorescence (FIG. 23E). The CpeB variants (C165S) and (C48S/C59S) were fluorescent due to the presence of covalently attached PEB (FIG. 23E, lane 1 and 3), while the CpeB (C80S) variant had no attached PEB (FIG. 23E, lane 2). After staining the same gel shown with Coomassie Blue, it was apparent that CpeB only accumulated in a soluble form when PEB had been ligated to it (FIG. 23D, lanes 1 and 3). The CpeB (C80S) variant produced in the presence of CpeS and PEB synthesis enzymes accumulated as the apoprotein in inclusion bodies as judged by immunoblotting. From these experiments, we conclude that the CpeS bilin lyase attaches PEB to Cys⁸° of CpeB, but it does not play a significant role in PEB attachment to CpeA.

Because CpeB does not accumulate in a soluble form in E. coli when no PEB is attached at Cys⁸⁰, we wondered whether CpeS was able to ligate a chromophore to any other site after it attached PEB at Cys⁸⁰. Recombinant CpeB-PEB (non-variant) produced with CpeS was subjected to trypsin digestion. The resulting tryptic peptides were separated by HPLC chromatography on a reversed-phase C18 column. In the chromatogram monitored at 550 nm to detect peptides with bound PEB, two peaks, eluting at 23 and 24 min, were observed and collected (See Supplemental FIG. 22). MALDI MS and tandem MS was used to identify the peptides from these two peaks. Supplemental FIG. 23A shows the MS/MS spectrum of the precursor at m/z 1250. The peaks at m/z 587 and 664 were most informative. The m/z 587 peak corresponds to protonated PEB as previously discussed. The peak at m/z 664 matched a peptide containing a cysteine at position 80, (R) MAACLR (D). The scheme in Supplemental FIG. 23B summarizes the structures of the assigned peaks. A review of the tandem mass spectra did not show an attachment to any other peptide. These results confirm that the CpeS bilin lyase specifically attaches PEB to Cys⁸⁰ of CpeB and to no other Cys residues.

### Analysis of the Ability of CpeY/CpeZ and CpeS to Attach Alternative Bilins to PE Subunits

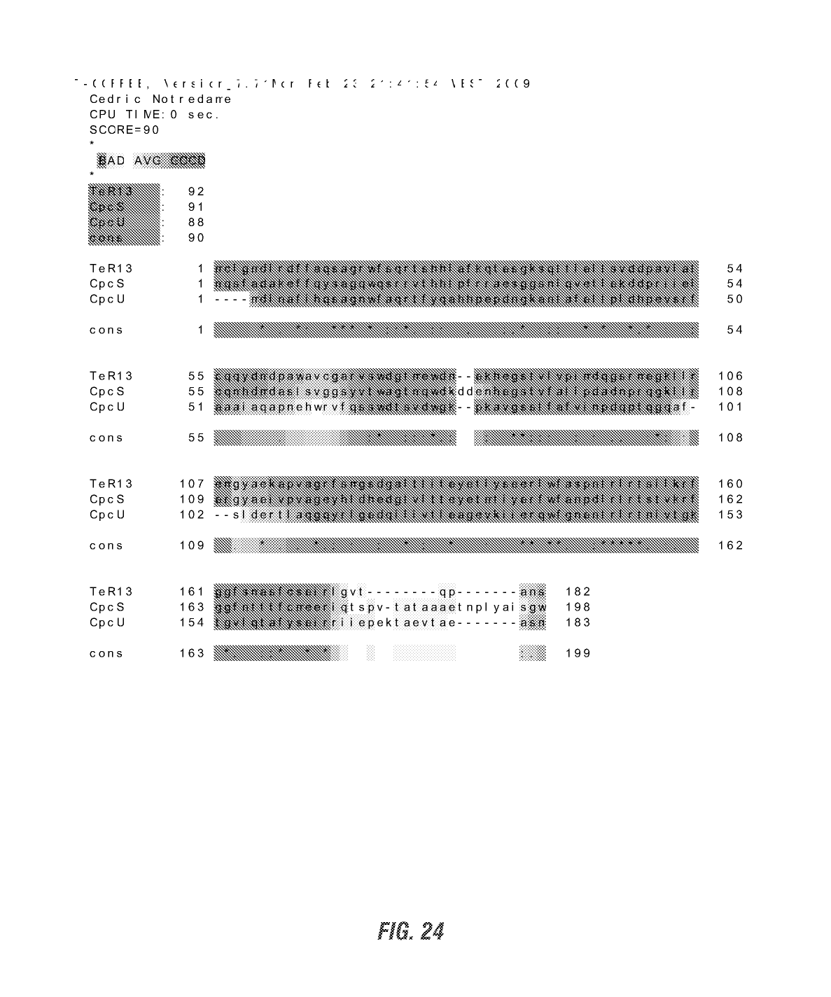

Because both PCB and PEB are synthesized in F. diplosiphon when cells are grown in green light, we tested whether the CpeY/CpeZ lyase could attach PCB to CpeA to determine the specificity of this lyase for bilin substrates. CpeA, which was produced in cells containing pPcyA and pCpeA with and without pCpeYZ, was analyzed by absorbance and fluorescence spectroscopy (Supplemental FIG. 24A). The CpeA produced in the absence of a lyase had no absorbance or fluorescence as expected. However, there was a small amount of absorbance and fluorescence of the CpeA product as a result of ligation of PCB to CpeA by CpeY/CpeZ. When these proteins were analyzed by SDS-PAGE (Supplemental FIG. 24B) and Zn-enhanced bilin fluorescence (Supplemental FIG. 24C), it was clear that although the amount of CpeA produced in these cells was very low, PCB was only attached to CpeA in the presence of the CpeY/CpeZ enzyme. Therefore, although this enzyme is capable of attaching PCB to CpeA, the amount of ligation was very low in comparison to its PEB ligation activity. When the ability of CpeS to ligate PCB to CpeB was tested in the same way, no absorbance or fluorescence was detected.

### Discussion

This study compared the activities of three putative bilin lyase subunits on CpeA and CpeB substrates derived from F. diplosiphon, a filamentous cyanobacterium capable of Type III complementary chromatic acclimation. This is the first examination of the bilin lyase specificity for both α and β-subunits of PE in which the enzymes and substrates were derived from the same organism. PEs are present in the distal ends of the peripheral rods of PBS, and they exhibit the most complex patterns of bilin binding sites that occur in PBPs. PE conjugates are also widely used as fluorescence markers for cell sorting, so an understanding of their biosynthesis could have biotechnological implications. The fluorescence quantum yield for the CpeY/CpeZ-generated CpeA was 0.72 and for the CpeS-generated CpeB-PEB was 0.89 (Table 1); most PE subunits (which may have two or three bilins attached) have quantum yields ranging between 0.84-0.98, which makes them excellent fluorescent markers.

Mass spectrometric data and attachment assays conducted with site-specific variants allowed confirmation of the site-specificity of CpeY/CpeZ for PEB ligation to Cys⁸² of the α-subunit, and the site-specificity of CpeS for linking PEB to Cys⁸⁰ of the β-subunit. Zhao et al. reported that CpcS from Nostoc sp. strain PCC 7120 had broad PBP substrate recognition and might attach all chromophores at position Cys⁸² except for those of CpcA, PecA, and RpcA. However, because Nostoc sp. PCC 7120 does not synthesize PE or PEB, a more thorough examination of the substrate specificity of bilin lyases for PE subunits within one organism seemed important. Other studies have shown that some bilin lyases are promiscuous with respect to both the bilin and PBP substrates. The studies reported here showed that CpeY/CpeZ, and not CpeS, is the principal bilin lyase responsible for attachment of PEB at Cys⁸² on CpeA. We did detect a small amount of PEB ligation at Cys¹³⁹ on CpeA using mass spectrometry, but the amount of attachment at Cys¹³⁹ in the presence of CpeY/CpeZ was very small compared to that at Cys⁸²; these data suggested that CpeY/CpeZ is not the lyase for this position. This conclusion is also supported by the fact that CpeY/CpeZ did not attach PEB to the CpeA (C80S) variant, whereas a small amount of PEB ligation by CpeS on the CpeA (C80S) variant was observed. While some PEB ligation to Cys¹³⁹ on CpeA by both CpeY/CpeZ and by CpeS was detected, the amounts were extremely low, and neither of these lyases seems likely to be responsible for PEB attachment at this Cys residue. Although CpeS could ligate PEB to Cys⁸² on CpeA, a comparison of the yields obtained with CpeS and CpeY/CpeZ proteins in E. coli strongly suggested that CpeY/CpeZ is more important in ligating PEB to CpeA to Cys⁸². Consistent with the data we show here, preliminary analyses of a F. diplosiphon cpeY deletion mutant, which avoids polarity effects that were likely present in the original cpeY transposon mutant isolated by Kahn et al., have shown that it produces very little PE in green light and that the PE that is synthesized has a defect in CpeA (Biswas, Gutu, Kehoe and Schluchter, unpublished data).

Wiethaus et al. showed that CpeS from Prochlorococcus marinus MED4 can ligate PEB to Cys⁸² of CpeB. This organism is unusual in the sense that cells are devoid of phycobilisomes, and furthermore they lack CpeA; the function of this degenerated form of CpeB is unknown. The F. diplosiphon CpeS bilin lyase is a polypeptide of 222 amino acids and is 42% similar to CpeS from Prochlorococcus marinus MED4, and it appears that both CpeS lyases are capable of ligating PEB to Cys⁸⁰ (equivalent) of CpeB. The studies reported here are the first to characterize a CpeS bilin lyase from a cyanobacterium containing PE in its phycobilisome rods. Unexpectedly, it was found that CpeS could also ligate PEB to Cys¹³⁹ of CpeA in addition to Cys⁸² on CpeA and Cys⁸⁰ on CpeB. However, based upon the very low levels of chromophorylation by CpeS at these positions, it seems unlikely that CpeS is the lyase that catalyzes these reactions in cyanobacteria. Several other bilin lyase candidates are currently being tested for PEB ligation at Cys¹³⁹ on CpeA.

At 429 amino acids, CpeY is much larger than typical members of the E/F lyase family, and it appears that it might have resulted from a fusion of ORFs encoded by a cpcE and cpcF-like gene. CpeY aligned well with the concatenated sequences of CpcE and CpcF of Synechocystis sp. strain PCC 6803 and with RpcG from Synechococcus WH8102. This could also explain why CpeY has significant activity in the absence of CpeZ. Individual CpcE and CpcF subunits usually exhibit low levels of ligation activity when assayed separately. For example, compared to PecE/PecF together, PecE from Mastigocladus laminosus had 10% PCB ligation activity on PecA. RpcG is also a larger bilin lyase that appears to have resulted from a fusion of genes encoding RpcE and RpcF; RpcG is involved in PEB chromophore ligation and isomerization to PUB on RpcA.

CpeZ is 205 amino acids in length and is most similar to CpcE-like, HEAT-repeat proteins that are found in cyanobacteria and in other bacteria that do not contain PBPs. All CpcE/CpcF-type bilin lyases contain 5-6 HEAT-repeat motifs; these motifs, which occur in many proteins in diverse eukaryotic organisms, are thought to facilitate protein-protein interactions. CpeZ increased the PEB ligation activity of CpeY, but no evidence for a stable interaction between CpeY and CpeZ was detected using pull-down assays. Likewise, no demonstrable interaction between CpeA and either CpeY or CpeZ was observed (data not shown). Nevertheless, CpeZ may play a chaperone-type role by assisting in the interactions of CpeA with other bilin lyases or with NblA/proteases.

Fairchild and Glazer produced recombinant apo-CpeA and apo-CpeB from F. diplosiphon in E. coli and found that both proteins were insoluble. They were able to partially renature CpeA but not CpeB from inclusion bodies, and they demonstrated some autocatalytic ligation of PEB to CpeA in vitro, but the spectrum of the product did not match that of native, holo-α-PE. When R-PE subunits were expressed in E. coli, they were also found to be insoluble unless fused to the maltose binding protein. In the experiments reported here, only a small amount of apo-CpeA was soluble, but PEB ligation increased its solubility. Co-expression of subunits of PC and AP increases their solubility in the apo-form, but co-expression of cpeB with cpeA without bilin attachment did not increase solubility (data not shown). However, chromophorylation at the position equivalent to Cys⁸² is obviously an important factor determining the solubility and accumulation of folded PBPs in E. coli. Although CpeB was slightly soluble at 18° C., we were unable to purify it in its apo-form. It became much more soluble when coexpressed with the CpeS lyase. Bilin deletion mutants in PC (where Cys were mutated to Ala) in cyanobacteria showed lower stability in vivo. The absence of bilins at various positions reduces the strength of α/β interactions in the heterodimers, and the authors suggested that these mutants were diverted to degradation pathways in cyanobacteria.

Because the CpcE/CpcF bilin lyases have been shown to have bilin removal activity, it seemed logical to test whether CpeY/CpeZ possessed such an activity. This assay is normally performed as a transfer assay using holo-PBP subunits and apo-PBP subunits that are either hexa-histidine tagged or bound to beads. Unfortunately, because apo-CpeA is not very soluble in E. coli, it was not possible to perform the PEB transfer assay. Both CpcE/CpcF and PecE/PecF subunits co-purify with their respective holo-PBP substrates, but CpeY (non-tagged) did not. This suggests that CpeY may not have a transferase activity.

Our mass spectrometry results build upon previous studies investigating the location of the attachment site of phycobilins. The PEB-peptide resulting from tryptic digestion of α-PE (detected at m/z 935) and from tryptic digestion of β-PE (detected at m/z 1250) were reported. Fragments resulting from tandem mass spectrometric experiments such as protonated free PEB (m/z 587), and the tri-pyrrole fragment that results from the loss of pyrrole ring D (m/z 464) were also reported. Using an off-line MALDI ToF/ToF method, very recently Wiethaus and coworkers were able to locate the site of ligation of PEB on the β-PE subunit from P. marinus based upon a major peak corresponding to PEB loss from a sequenced tryptic peptide containing Cys⁸².

The molecular structure of the tetrapyrrole, PEB, has an extended “π-conjugated” system, and may exist as three different tautomers. These isomeric structures, which exist in a dynamic equilibrium, differ only in that the pyrrole rings carrying the imino and amino nitrogens have changed. FIG. 19, shows structures of PEB attached to tryptic peptides derived from CpeA and CpeB. The tautomeric forms shown are based upon previously published work that relied upon NMR to assign the predominant tautomer.

Why are different lyases needed for ligation of bilins to Cys⁸² of the α and β subunits of PBPs that occur in the rods of PBS (e.g., CpcA/CpcB, PecA/PecB and CpeA/CpeB)? Because lyases of the CpcS/CpcU family are capable of chromophorylating both β and α subunits such as ApcA, ApcB, CpcB, and minor AP subunits, it seems plausible that CpcA was originally chromophorylated by a lyase of this type. Therefore, it seems likely that the CpcE/CpcF lyase family evolved later, perhaps to perform a specialized function. For example, some members of the E/F family of bilin lyases can remove and transfer bilins, and the α-subunits of PBP have been shown to interact with NblA near the binding site for the chromophore at Cys⁸² of the PC α-subunit. Thus, it has been speculated that these lyases might have a unique role in biliprotein degradation/turnover during nutrient starvation conditions. Another possibility is that the evolution of organisms producing PEs and PEBs required greater specificity in the existing lyases, especially those involved in the core energy transfer reactions (i.e., ApcA, ApcB, and CpcB), in order to prevent the mis-attachment of PEB at sites that would greatly reduce energy transfer efficiency. The postulated greater specificity may have come at the expense of slow chromophore ligation on some apo-proteins, e.g., CpcA. Together with the necessity to add PEB chromophores to the apo-PE subunits, cyanobacteria evolved new lyases to accommodate the greater complexity of chromophore-substrate possibilities. Whatever the true origin of this lyase specialization, once it occurred, nature took advantage of this to diversify PCs by gene duplication and divergence. In extant cyanobacteria, all four known chromophores (PCB, PEB, PUB, and phycoviolobilin) can occur at Cys⁸² in the α subunits of peripheral rod proteins. The production of two of these chromophores, phycoviolobilin and PUB, requires a bilin lyase/isomerase, like PecE/PecF or RpcG, a capability that evolved by duplication and divergence from the CpcE/CpcF lyases. Finally, chromophores at the Cys⁸² position of α-subunits transfer energy to the terminal acceptor bilin present at Cys⁸² on β-subunits within trimers (αβ)₃, so there appears to be more flexibility for differences in chromophore type on the α-subunits. Although this is not a reason why a specialized lyase evolved, it may have facilitated such a development.

The fluorescence quantum yields of CpeA-PEB and CpeB-PEB were 0.72 and 0.89, respectively; these values are much higher than the quantum yield of 0.60 obtained from the best mutant of green fluorescent protein (GFP). They are also larger than the values reported for CpeA-PEB (0.51) and CpeB-PEB (0.63) produced in E. coli with CpcS1. Therefore, these recombinant proteins may be useful for biotechnological applications as fluorescent probes or for therapeutic purposes, because R-PE has been used a photosensitizer in cancer cells. Finally, with this report and that of Wiethaus et al., details of how these complex yet important PEs are biosynthesized are finally emerging. However, many unresolved questions remain. What is the exact role of CpeZ in PE biosynthesis? Which bilin lyases ligate PEB to the other Cys residues, including α-Cys¹³⁹, β-Cys^{48,59} and β-Cys¹⁶⁵? We are approaching these questions by using a combination of a reverse-genetics approach and biochemical analyses of recombinant proteins, and answers should soon be forthcoming.

TABLE 3
Comparison of spectral properties for various PE subunits produced with bilin lyases

| Plasmids used for | Plasmids used for | λₘₐₓ (nm) | Fluorescence | | % Fluorescence |
|---|---|---|---|---|---|
| Apo-proteins | bilin lyases | (Q_{Vis/UV})² | Emission λₘₐₓ (nm) | Φ_{f} | emission |
| pCpeA | pCpeYZ¹ | 566/410 (5.4) | 574 | 0.72 | 100 |
| pCpeA(C82S) | pCpeYZ¹ | NA⁴ | NA | ND⁵ | ND⁵ |
| pCpeA(C139S) | pCpeYZ¹ | 566/410 (5.6) | 574 | ND | ND |
| pCpeA(C82S, 139S) | pCpeYZ¹ | NA | NA | ND | ND |
| pCpeA | pCpeZ¹ | NA | NA | ND | 0 |
| pCpeA | pCpeY¹ | 566/410 (15.6) | 574 | ND | 60 |
| pCpeA(C82S) | pCpeY¹ | NA | NA | ND | ND |
| pCpeA(C139S) | pCpeY¹ | 566/410 (14.8) | 574 | ND | ND |
| pCpeA(C82S, 139S) | pCpeY¹ | NA | NA | ND | ND |
| pCpeA | pCpeS¹ | 561/410 (0.315) | 574 | 0.89 | 0.8 |
| pCpeA(C82S) | pCpeS¹ | 550/398 (0.6) | 562 | ND | ND |
| pCpeA(C139S) | pCpeS¹ | 564/410 (0.6) | 574 | ND | ND |
| pCpeA(C82S, 139S) | pCpeS¹ | NA | NA | ND | ND |
| pCpeB | pCpeS¹ | 560/412 (5.2) | 571 | ND | ND |
| pCpeB(C80S) | pCpeS¹ | NA | NA | ND | ND |
| pCpeB(C165S) | pCpeS¹ | 560/412 (5.4) | 571 | ND | ND |
| pCpeB(C48S, 59S) | pCpeS¹ | 560/398 (5.3) | 571 | ND | ND |
| Native PE (αβ)₆ ³ | | 563/374 (9.5) | 573 | ND | ND |

¹Coexpressed with pPebS
²Q_{Vis/UV} denotes the absorbance ratio of the visible and near-UV bands
³ Holo PE purified from F. diplosiphon.
⁴Not applicable since there was no fluorescent product produced
⁵Not determined

TABLE 4
Plasmids used in this study

| Plasmid Name | Recombinant proteins produced^{a} | Parent vector | Antibiotic^{b} | Reference |
|---|---|---|---|---|
| pPebS | Myovirus HO1 and HT-PebS | pACYCDuet-1 | Cm | (1) |
| pPcyA | PcyA from Synechococcus sp. PCC | pACYCDuet-1 | Cm | (2) |
| | 7002 and Ho1 from Synechocystis sp. | | | |
| | PCC 6803 | | | |
| pCpeA | F. diplosiphon HT-CpeA | pETDuet-1 | Ap | This paper |
| pCpeA: C82S | F. diplosiphon HT-CpeA | pETDuet-1 | Ap | This paper |
| | (Cys⁸² mutated to Ser) | | | |
| pCpeA: C139S | F. diplosiphon HT-CpeA | pETDuet-1 | Ap | This paper |
| | (Cys¹³⁹ mutated to Ser) | | | |
| pCpeA: C82S/C139S | F. diplosiphon HT-CpeA | pETDuet-1 | Ap | This paper |
| | (Cys⁸² and Cys¹³⁹ mutated to Ser) | | | |
| pCpeB | F. diplosiphon HT-CpeB | pETDuet-1 | Ap | This paper |
| pCpeB: C80S | F. diplosiphon HT-CpeB | pETDuet-1 | Ap | This paper |
| | (Cys⁸⁰ mutated to Ser) | | | |
| pCpeB: C165S | F. diplosiphon HT-CpeB | pETDuet-1 | Ap | This paper |
| | (Cys¹⁶⁵mutated to Ser) | | | |
| pCpeB: C48S/C59S | F. diplosiphon HT-CpeB | pETDuet-1 | Ap | This paper |
| | (Cys⁴⁸ and Cys⁵⁹ mutated to Ser) | | | |
| pCpeZ | F. diplosiphon, HT-CpeZ | pCOLADuet-1 | Km | This paper |
| pCpeY | F. diplosiphon CpeY | pCOLADuet-1 | Km | This paper |
| pCpeYZ | F. diplosiphon HT-CpeZ and CpeY | pCOLADuet-1 | Km | This paper |
| pCpeS | F. diplosiphon CpeS | pCOLADuet-1 | Km | This paper |

^{a}Proteins produced as Hexa-histidine-tagged fusions are indicated as HT-
^{b}Antibiotic resistance used to select for the presence of the plasmid (Ap: ampicillin, Cm: chloramphenicol; Km: kanamycin; Sp: spectinomycin)
(1). Danmeyer, T., Bagby, S. C., Sullivan, M. B., Chisholm, S. W., and Frankenberg-Dinkel, N. (2008) Curr. Biol. 18, 442-448
(2). Biswas, A., Vasquez, Y. M., Dragomani, T. M., Kronfel, M. L., Wiliams S. R., Alvey, R. M., Bryants, D. A., and Schluchter, W. M. (2010) Appl. Environ. Microbiol. 76, 2729-2739.

### Example 5

### Structural and Biochemical Characterization of the Bilin Lyase CpcS from Thermosynechococcus elongatus

### Materials and Methods

### Construction of Expression Vectors

Most of the plasmids used in this study have previously been described and are listed in Table 5. The cpcS gene from T. elongatus (accession number Q8DI91) was amplified by PCR using primers TEcpcSF (SEQ ID NO:22)(5′-tccccattagCATATGtgcataggtatggacatccgc-3′, added NdeI site in capital letters) and TEcpcSR(SEQ ID NO:23) (5′-gaaaaaCTCGAGggagttggcgggttgcgtc-3′, added XhoI site in capital letters), digested with NdeI and XhoI, and ligated into similarly digested pCOLADuet-1 (Novagen, Madison, Wis.). Recombinant CpcS expressed from this plasmid contains a C-terminal S-tag. For some experiments such as for crystallization, and for purification for size-exclusion chromoatography, the cpcS gene was cloned in pET22c with a C-terminal His-tag.

### Expression and Purification of Recombinant Proteins:

Plasmids were co-transformed into E. coli BL21(DE3), and cells grown on Luria Bertani (LB) medium containing the appropriate antibiotics for selection as listed in Table 5 at the following concentrations: ampicillin: 100 μg·ml⁻¹; chloramphenicol: 34 μg·ml⁻¹: kanamycin: 50 μg·ml⁻¹; and/or spectinomycin: 100 μg·ml⁻¹. Isolated colonies were used to inoculate 50 ml cultures of LB with necessary antibiotics combinations at the aforementioned concentrations and grown at 37° C. with shaking at 225 rpm before transfer to 1 L LB medium plus antibiotics for incubation at same conditions for until optical density OD_{600 nm}=0.6 (approximately 4 h). The temperature was lowered to 30° C. before induction of T7 RNA polymerase by 1 mM isopropyl β-D thioglactoside (IPTG) and incubation was continued an additional 4 h. Cells were collected by centrifugation at 10,000×g for 10 min and stored at −20° C. until required. For PEB production using pPebS expression construct the culture was induced at 18° C. with 1 mM IPTG for another 16 h, before being harvested.

Cells were thawed and resuspended in a buffer of 50 mM Tris-HCl, 150 mM NaCl, pH 8.0 at 2.5 ml buffer per g wet-weight and lysed by three passages through a chilled French pressure cell at 138 MPa. Inclusion bodies, cell debris, and unbroken cells were removed by centrifugation for 20 minutes at 13,000×g. The supernatant containing soluble proteins was applied to a nickel-nitrilotriacetic acid superflow affinity column (Qiagen, Inc., Chatsworth, Calif.) as previously described for purification of hexa-histadine tagged proteins and any interacting products. Imidazole introduced during elution was removed by overnight dialysis with suspension buffer plus 10 mM 2-mercaptoethanol.

### Fluorescence Emission and Absorbance Spectra:

Fluorescence emission and excitation spectra were recorded with a Perkin Elmer LS55 fluorescence spectrophotometer (Waltham, Mass.) with slit widths of 10 nm. The excitation wavelength was set at 490 nm for recombinant proteins co-expressed with PEB and at 590 for those with PCB or PΦB. Chromophorylated samples were diluted to ˜0.05 OD (at λₘₐₓ) prior to obtaining their fluorescence spectra to accommodate the sensitivity range of the machine. Samples produced in the absence of a lyase, which had very little or no chromophore attached, were measured without dilution because their OD was generally less than 0.05 at the original concentration. Absorbance spectra were acquired using a lambda 35, dual-beam UV/Vis spectrophotometer (Perkin Elmer, Waltham, Mass.). For calculating relative fluorescence intensities, samples were diluted to 0.05 OD_{560 nm}.

### Protein and Bilin Analysis:

Polypeptides were resolved by polyacrylamide gel electrophoresis (PAGE; 15% w/v) in the presence of sodium dodecyl sulfate (SDS), and polypeptides were visualized by staining with Coomassie blue as described. To detect proteins containing bound chromophores (PEB, PCB or PΦB), gels were soaked in 100 mM ZnSO₄. The resulting Zn-enhanced fluorescence produced by chelation of the bilin could be visualized using an FX imaging system (BioRad, Hercules, Calif.) with excitation at 550 nm.

### Results

### Structural Analysis of TeCpcS.

The X-ray crystal structure of CpcS-III (hereafter TeCpcS) from Thermosynechococcus elongatus BP1 (tll1699) was very recently solved at 2.9 Å resolution, and the coordinates were entered into the PDB database. It shares sequence similarity with other PBP lyases, including CpcS from Nostoc sp. PCC 7120, CpeS from F. diplosiphon, and heterodimeric CpcS/CpcU from Synechococcus sp. PCC 7002. This structure was solved as part of a structural genomics project and represents the first structure of a bilin lyase. TeCpcS crystallized as a dimer and belongs to the lipocalin structural family; it is a 10-stranded antiparallel β-barrel with two α-helices. Lipocalins have various oligomeric states, occurring as monomers, homodimers, heterodimers, or tetramers, and they bind a diverse set of ligands including fatty acids, retinols, carotenoids, pheromones, prostaglandins, and biliverdin. TeCpcS crystallized as a dimer. The phosphate ion bound by the protein probably interacts with Arg residues that normally bind the propionic acid side chains of the PCB. The most similar structure to TeCpcS in the protein structure database is the bilin-binding protein of the insect Pieris brassicae. This insect protein binds biliverdin IXγ and is responsible for coloration of various stages of some insects, most commonly in members of the Lepidoptera. Using the structure of the bilin-binding protein and its interaction with a different isomer biliverdin IXγ as a guide, the structure of PCB bound to TeCpcS was modeled.

TeCpcS is 182 amino acids in length and is related in sequence to CpcS and CpcU from Synechococcus sp. PCC 7002. These two proteins form a heterodimeric bilin lyase that can attach PCB at Cys-82 of all types of AP subunits (ApcA, ApcB, ApcD, ApcF) and CpcB [12, 25]. When TeCpcS was aligned with the sequences of CpcS and CpcU, the sequences were highly conserved until the extreme C-terminus (see FIG. 24). TeCpcS is the only CpcS-type bilin lyase found in the T. elongatus genome. Therefore, it was important to determine whether TeCpcS had bilin lyase activity on phycobiliprotein subunits and to explore its versatility for bilin binding and attachment to allow for the creation of unique phycobiliproteins.

Analysis of Lyase Activity of TeCpcS with CpcB

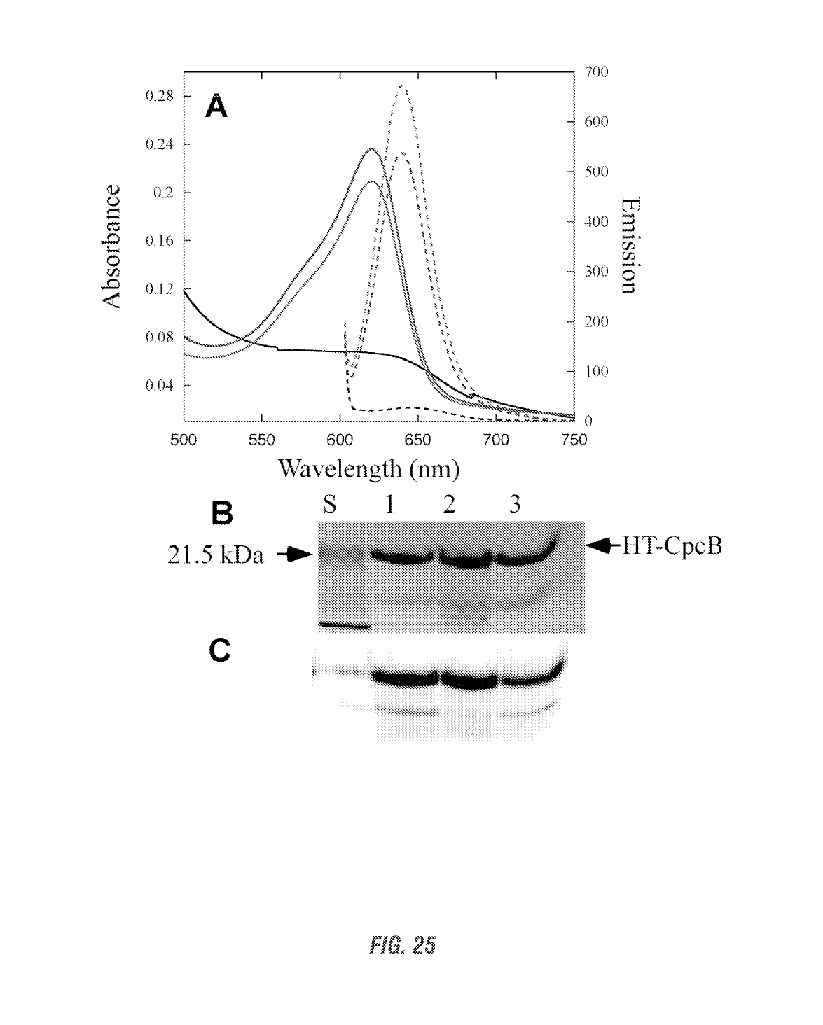

TeCpcS is the first bilin lyase whose structure was resolved at 2.9⁰ Å resolution. Thus, it was important to test the lyase activity of this protein on various apo-PBP subunits using the heterologous coexpression system in E. coli. The purified PBPs obtained from the E. coli cells harboring coproducing CpcBA, TeCpcS, and PcyA (See Material and Methods) were analyzed using absorbance and fluorescence spectroscopy. FIG. 25A shows the absorbance (solid blue line) and fluorescence emission (dotted blue line) spectra of PCB ligated to CpcB by TeCpcS. The black solid and dotted lines correspond to absorbance and fluorescence spectra, respectively for purified CpcB obtained without TeCpcS. To compare the lyase activity of TeCpcS with that of the CpcS/CpcU lyase, which was previously shown to chromophorylate CpcB at Cys82, CpcB was coproduced with CpcS, CpcU, and PcyA. In FIG. 25A, the red solid line represents the absorbance spectrum with an absorbance maxima at 620 nm (see Table 5), and the red dotted line represents the fluorescence emission spectrum with an emission maximum at 644 nm (See Table 5) for purified CpcB obtained by coproduction of CpcBA, CpcS, CpcU, and PcyA. The absorption and fluorescence emission spectra were nearly identical, confirming that TeCpcS has the same lyase activity as CpcS/CpcU (See Table 5). The absorbance and fluorescence properties of PCB ligation at Cys-153 (592 nm absorbance and 624 nm fluorescence emission) are distinct from ligation at Cys-82 (absorbance at 621 nm and fluorescence emission at 644 nm; see Table 5), and therefore, it is concluded that TeCpcS ligates PCB at Cys-82 on CpcB as expected.

The purified, PCB-containing CpcB proteins were separated by SDS-PAGE to analyze the proteins present by Coomassie Blue staining (FIG. 25B). The amount of CpcA expressed in these cultures was low (it can vary widely), and CpcA is not visible in these gels. TeCpcS was tested with an expression construct for HT-CpcA separately, and no covalent bilin addition was detected (data not shown). The amount of covalent bilin addition to CpcB was verified by Zn-enhanced fluorescence emission of the same gel as shown in FIG. 25C. The strong Zn-enhanced fluorescence emission observed for the proteins in lanes 1 and 3 indicates that TeCpcS ligated PCB to CpcB. A similar result was obtained when the lyase was CpcS/CpcU (FIG. 25C, lane 2). From these experiments, it can be concluded that TeCpcS acts as an S-type lyase that ligates PCB to CpcB at Cys-82. These results establish that the TeCpcS structure is that of a functionally active bilin lyase.

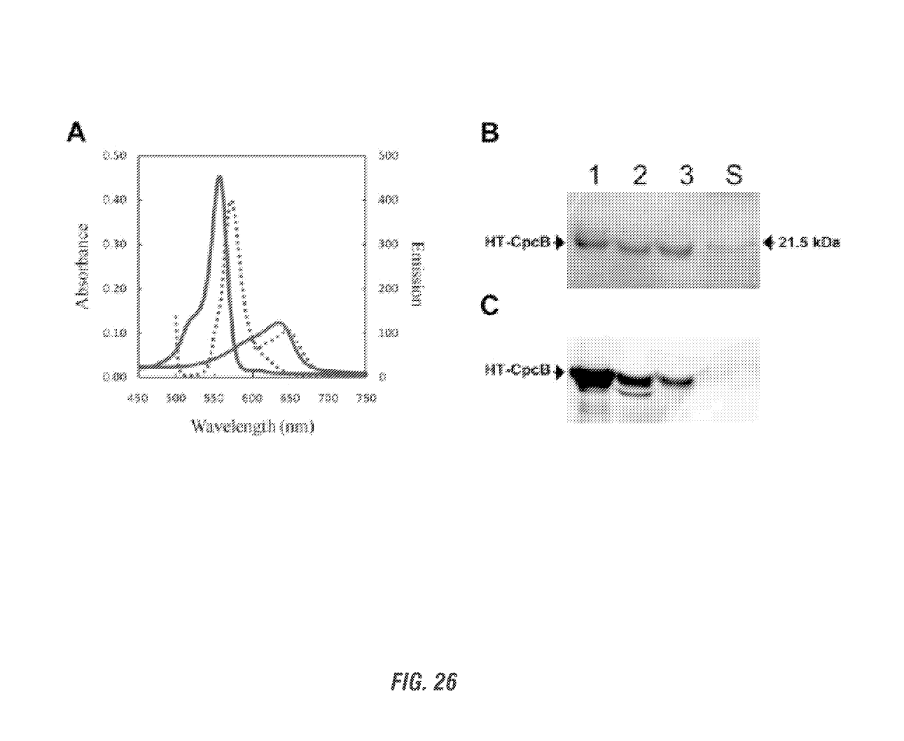

The ability of TeCpcS to attach other non-cognate bilins to CpcB was tested by coproducing CpcB and TeCpcS with either PebS or HY2. As shown in FIG. 26A, TeCpcS could attach PEB (solid red lines for absorbance, dotted red lines for fluorescence emission) and PΦB (solid green lines for absorbance and dotted green lines for fluorescence) to CpcB to produce highly fluorescent products with interesting spectral properties (see Table 5). These samples were separated by SDS-PAGE (FIG. 26B) and stained with Coomassie Blue (FIG. 26B). The bilin content was examined by Zn-enhanced fluorescence of the same gel as shown in FIG. 26C. The results in FIG. 26C showed that CpcB had covalently bound PEB (lane 1), PΦB (lane 2), and PCB (lane 3).

### Analyzing Activity of TeCpcS Lyase on Major Allophycocyanin Subunits ApcA/ApcB

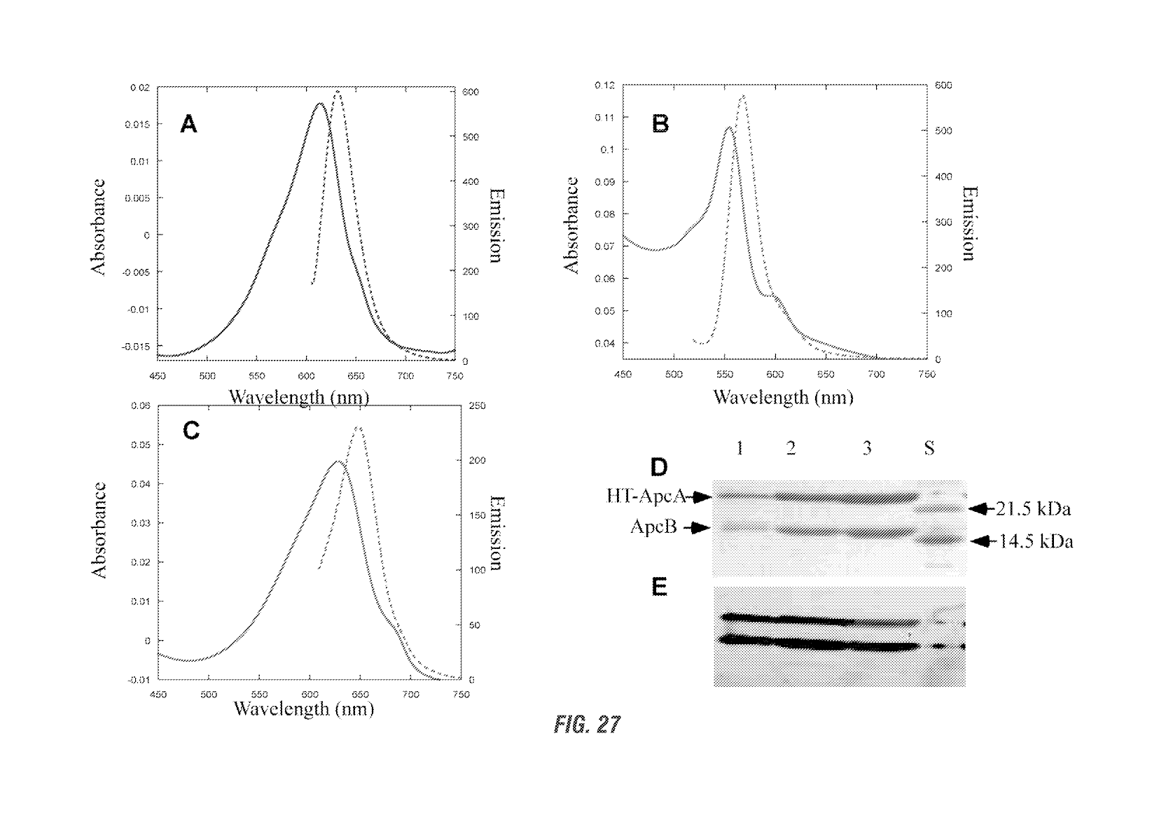

The heterodimeric CpcS/CpcU lyase can chromophorylate all AP subunits at the Cys82-equivalent position, so the TeCpcS was tested for its ability to chromophorylate ApcA and ApcB. The TeCpcS lyase activity was tested with different types of bilin substrates (PCB, PEB or PΦB), although PEB and PΦB are non-cognate bilins for T. elongatus. The ApcA and ApcB subunits were coproduced with TeCpcS in cells producing PCB, PEB, and PΦB (See Table 3). The expressed proteins were purified using metal affinity chromatography. The purified proteins were analyzed by absorbance and fluorescence emission spectroscopy, and the data are shown in FIG. 27. The solid lines show the absorbance spectra, and the dotted lines show the fluorescence emission spectra of the purified ApcA and ApcB ligated with PCB (FIG. 27A) or PEB (FIG. 27B) or PΦB (FIG. 27C). The absorbance and fluorescence emission maxima as summarized in Table 5 show proper addition of PCB as established in previously published studies. As previously reported for CpcS/CpcU, no significant bilin ligation occurred in absence of TeCpcS lyase (data not shown). To our knowledge, this is the first report of PEB and PΦB ligation to AP subunits. The strong absorbance and fluorescence emission of the PEB and PΦB variants as seen in FIGS. 27B and 27C is strong evidence that these bilins have been added to the correct Cys82 residues (see Table 5). To verify that the chromophores were covalently bound to both ApcA and ApcB, the purified proteins were separated on SDS-PAGE (FIG. 27D) and stained with Zinc sulphate to confirm bilin addition on both ApcA and ApcB (FIG. 27E). Both subunits were fluorescent when produced in the presence of PCB, PEB, or PΦB as seen in FIG. 27E indicating the bilin was covalently ligated to each subunit (lanes 1 through 3). These data establish that TeCpcS is a novel, S-type lyase that can ligate PCB, PEB and PΦB to both ApcA and ApcB. The CpcS/CpcU lyase shows low bilin ligation activity with PEB compared to TeCpcS, but CpcS/CpcU was shown to be able to efficiently attach PΦB in cyanobacteria.

### TeCpcS Activity on AP α-Like Subunit ApcD

ApcD is a variant AP alpha subunit (α^{AP-B}) that pairs with ApcB to form AP-B, which has an extremely red-shifted absorbance at 670 nm. AP-B and is an important terminal emitter of the PBS, which is involved in energy transfer to Photosystem I, and is present in two copies per PBS. The CpcS/CpcU lyase has the ability to ligate PCB to apo-ApcD in E. coli. When expressed along with ApcB, this increases the solubility of ApcD and energy transfer from the bilin on ApcB to ApcD was observed in the recombinant protein.

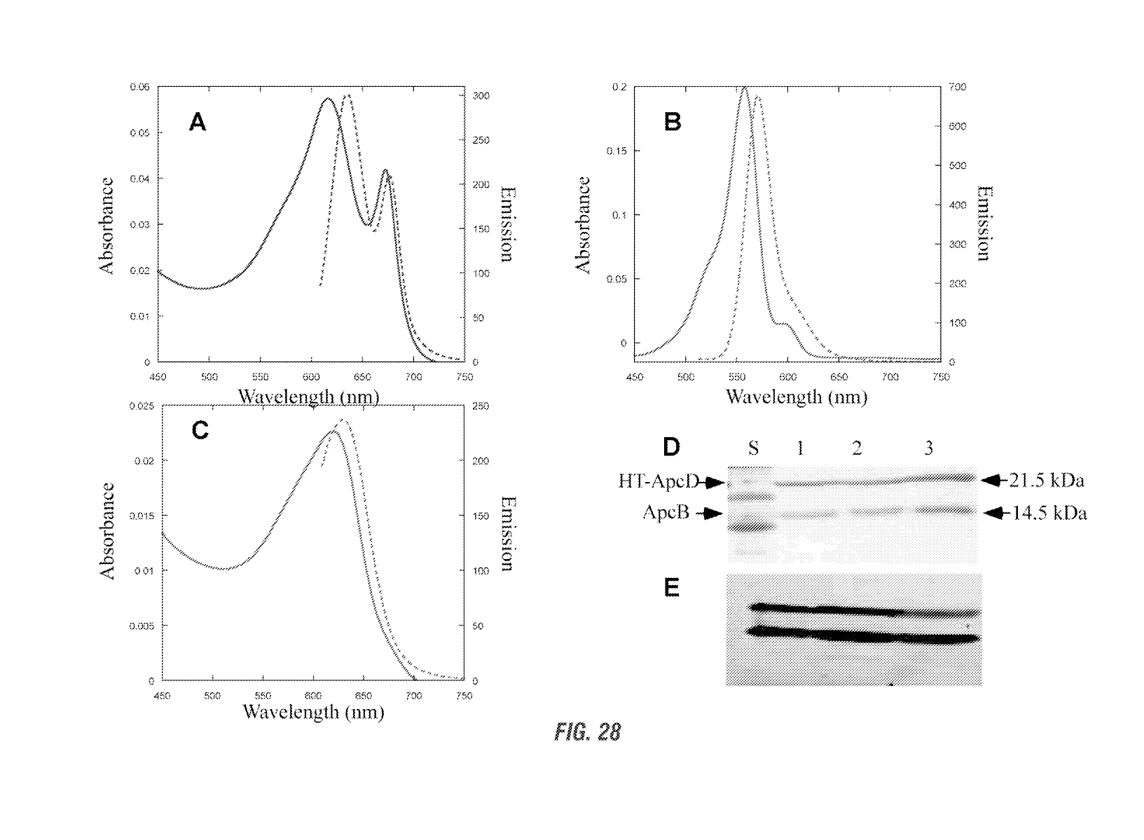

To test the TeCpcS bilin lyase activity on ApcD, TeCpcS was coexpressed with ApcD and ApcB together with one of the three bilins (PCB, PEB or PΦB). These resulting cells were found to be intensely colored (data not shown), suggesting that covalent bilin ligation had occurred. The His-tagged proteins were purified from cells using Ni-NTA column chromatography. The purified proteins were characterized as described above using absorbance and fluorescence emission spectroscopy. The absorbance (solid lines) and fluorescence emission (dashed lines) in FIGS. 28 A, B, and C show the results for ApcD/ApcB ligated with three different bilins: PCB (FIG. 28A), PEB (FIG. 28B), PΦB (FIG. 28C) (see Table 5). The properties of the PCB-ligation on ApcD protein were similar to those previously reported. It is interesting that only when PCB was ligated to these proteins did the longer wavelength absorption band characteristic of native ApcD appear. To show that the chromophores were covalently bound to both ApcD and ApcB, the purified proteins were separated on SDS-PAGE and stained with Coomassie Blue (FIG. 28D) and with zinc sulphate (FIG. 28E, lanes 1-3). Zn-enhanced fluorescence showed that the chromophores were covalently attached to both ApcB and ApcD. Therefore, TeCpcS can attach PCB, PEB, and PΦB to ApcD. Naturally, ApcD only harbors only PCB and acts as a terminal energy acceptor.

### TE CpcS Activity on ApcF

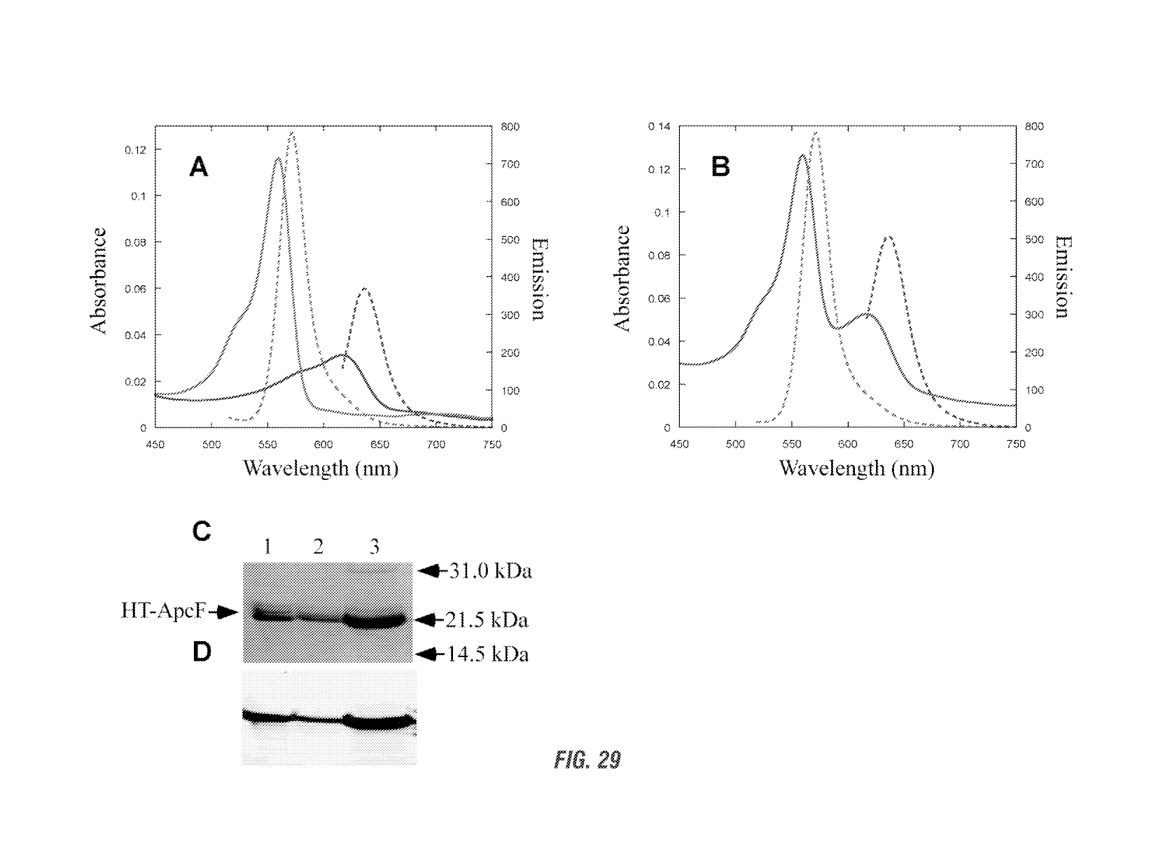

ApcF is a variant type of AP β subunit (also known as β¹⁸) that partners with ApcE (the core membrane linker PBP subunit), the second terminal emitter of PBS, and in Synechococcus sp. strain PCC 7002 the loss of ApcF affects energy transfer from PBS to photosystem II. Previously published data showed that the heterodimeric CpcS/CpcU lyase can ligate PCB on ApcF. TeCpcS was coproduced with ApcF together with enzymes to produce three bilins (PCB, PEB or PΦB). The resulting cells were found to be intensely colored (data not shown), which suggested that efficient ligation of chromophores to ApcF had occurred. The proteins were purified using metal affinity chromatography. In FIG. 29 A, B, the solid lines represent absorbance and dotted lines represent the fluorescence emission spectra of ApcF carrying two different bilins, PCB and PEB (See Table 5). The purified proteins were separated on SDS-PAGE and stained with Coomassie blue for protein (FIG. 28D) and stained with zinc sulphate to confirm bilin addition to ApcF (FIG. 28E, lanes 1 through 3). TeCpcS was able to attach PCB and PEB to ApcF, even though ApcF naturally only binds PCB. When both PCB and PEB were produced in E. coli along with ApcF and TeCpcS, TeCpcS preferentially attached the non-cognate bilin, PEB (FIG. 29B). T. elongatus PBPs naturally contain only PCB, and it may be that there is little need for TeCpcS to discriminate among bilins in this organism. When the same experiment was done with CpcS/CpcU and ApcF, CpcS/CpcU showed more specificity in attaching PCB over PEB which may be an advantage in using this protein for FRET studies. Synechococcus sp. PCC 7002 may have had evolutionary ancestors that contained PEB, which may have produced a greater need to discriminate between PCB and PEB. This might explain why the CpcS/CpcU lyase cannot utilize PEB as a substrate as readily as TeCpcS.

### Intrinsic Bilin Binding Property of TeCpcS

Similar to other CpcS and CpcS/CpcU lyases, the purified TeCpcS obtained by producing the protein together with PcyA or PebS was weakly fluorescent. The fluorescence intensity was much lower than that obtained with PBP subunits. When separated on SDS-PAGE and stained with Coomassie blue or with Zn to enhance bilin fluorescence both TeCpcS-PCB and TECpcS-PEB had Zn-enhanced fluorescence emission bands indicating that TeCpcS can ligate either PCB or PEB forming a low yield fluorescent adduct.

### Investigation of the Molecular Mass of the TeCpcS Protein

In order to determine the molecular mass of TeCpcS in its native state, the purified, recombinant TeCpcS was subjected to size exclusion chromatography. The molecular mass of this complex was calculated to be 53.0 kDa, and the calculated molecular mass of HT-TeCpcS is ˜24.0 kDa. These data suggest that the protein is stable and active as a homodimer in its native state, which is consistent with the observation that TeCpcS crystallizes as a dimer.

The overall conclusion of this study is that we have characterized a new bilin lyase, which functions as a dimer and which shares 50-60% sequence similarity with known CpcS type lyases. TeCpcS can ligate cognate and non-cognate bilins to all AP subunits and to CpcB. The X-ray crystal structure of this lyase may be helpful in using this lyase to design various fluorescent tag-proteins for biotechnological applications. TeCpcS and CpcS/CpcU show the same phycobiliprotein substrate preference but appear to have different abilities to attach the non-cognate bilin PEB.

TABLE 5
Spectral properties for PC and AP subunits
with cognate and non-cognate bilins

| | | Fluorescence |
|---|---|---|
| Holo recombinant PBPs | λₘₐₓ (nm) | Emission |
| (Plasmid present) | (Q_{Vis/UV}) | λₘₐₓ (nm) |
| HT-CpcB (pCpcBA + pPcyA)¹ | 620/394 (4.3) | 644 |
| HT-CpcB (pCpcBA + pPcyA)² | 621/393 (4.7) | 644 |
| HT-CpcB (pCpcBA + pPebS¹ | 557/372 (10.64) | 573 |
| HT-CpcB (pCpcBA + pHY2)¹ | 635/347 (2.25) | 647 |
| HT-ApcA/ApcB (pApcAB + | 614/392 (5.3) | 632 |
| pPcyA)¹ | | |
| HT-ApcA/ApcB (pApcAB + | 560/376 (8.3) | 571 |
| pPebS)¹ | | |
| HT-ApcA/ApcB (pApcAB + | 629/391 (4.8) | 648 |
| pHY2)¹ | | |
| HT-ApcD (pApcDB + pPcyA)¹ | 672/370 (3.2) | 672 |
| HT-ApcD (pApcDB + pPebS)¹ | 572/371 (2.8) | 571 |
| HT-ApcD (pApcDB + pHY2)¹ | 629/391 (2.4) | 648 |
| HT-ApcF (pApcF + pPebS)¹ | 560/376 (8.1) | 572 |
| HT-ApcF (pApcF + pPcyA)¹ | 615/393 (4.2) | 632 |
| HT-ApcF(pApcF + pHY2)¹ | 624/391 (1.5) | 645 |

¹The constructs were coexpressed with TeCpcS
²The constructs was coexpressed with pCpcSU

```
Amino acid sequence of Te CpcS also designated as tll1699 and as Ycf58
    1 mcigmdirdf faqsagrwfs qrtshhlafk qtesgksqlt iellsvddpa vialcqqydm
  61 dpawavcgar vswdgtmewd nekhegstvl vpimdqgsrm egkllremgy aekapvagrf
121 smgsdgaltl iteyetiyse erlwfaspnl rlrtsilkrf ggfsmasfcs eirlgvtqpa
181 ns
```

Gene Sequence from Genbank Including the Position within the Genome:

>gi|22297544:c1780924-1780376 Thermosynechococcus elongatus BP-1,
complete genome
GTGTGCATAGGTATGGACATCCGCGATTTTTTTGCCCAAAGTGCTGGCCGCTGGTTTTCCCAACGGACGA
GTCATCACTTGGCCTTTAAGCAAACCGAGTCCGGCAAGTCTCAGTTAACCATTGAATTACTGTCGGTGGA
TGATCCTGCAGTCATTGCCCTGTGTCAGCAATATGATATGGATCCCGCTTGGGCAGTGTGCGGCGCGCGG
GTCAGTTGGGACGGCACCATGGAATGGGACAATGAGAAGCACGAAGGCTCAACAGTACTGGTACCCATCA
TGGATCAGGGATCGCGGATGGAAGGAAAGCTCCTGCGGGAAATGGGCTACGCTGAAAAAGCACCGGTTGC
CGGTCGCTTCAGCATGGGCAGTGATGGTGCCCTGACATTGATTACCGAATACGAAACCATCTATTCCGAA
GAGCGCCTCTGGTTTGCCAGCCCCAATTTACGTCTGCGCACCAGTATTCTCAAGCGCTTTGGTGGTTTTA
GTATGGCCTCCTTCTGCTCGGAAATTCGCTTAGGGGTGACGCAACCCGCCAACTCCTGA

```
Amino acid sequence of Te CpcS also designated as tll1699 and as Ycf58
(SEQ ID NO: 24)
    1 mcigmdirdf faqsagrwfs qrtshhlafk qtesgksqlt iellsvddpa vialcqqydm
  61 dpawavcgar vswdgtmewd nekhegstvl vpimdqgsrm egkllremgy aekapvagrf
121 smgsdgaltl iteyetiyse erlwfaspnl rlrtsilkrf ggfsmasfcs eirlgvtqpa
181 ns
```

Gene Sequence from Genbank Including the Position within the Genome:

>gi|22297544:c1780924-1780376 Thermosynechococcus elongatus BP-1,
complete genome
(SEQ ID NO: 25)
GTGTGCATAGGTATGGACATCCGCGATTTTTTTGCCCAAAGTGCTGGCCGCTGGTTTTCCCAACGGACGA
GTCATCACTTGGCCTTTAAGCAAACCGAGTCCGGCAAGTCTCAGTTAACCATTGAATTACTGTCGGTGGA
TGATCCTGCAGTCATTGCCCTGTGTCAGCAATATGATATGGATCCCGCTTGGGCAGTGTGCGGCGCGCGG
GTCAGTTGGGACGGCACCATGGAATGGGACAATGAGAAGCACGAAGGCTCAACAGTACTGGTACCCATCA
TGGATCAGGGATCGCGGATGGAAGGAAAGCTCCTGCGGGAAATGGGCTACGCTGAAAAAGCACCGGTTGC
CGGTCGCTTCAGCATGGGCAGTGATGGTGCCCTGACATTGATTACCGAATACGAAACCATCTATTCCGAA
GAGCGCCTCTGGTTTGCCAGCCCCAATTTACGTCTGCGCACCAGTATTCTCAAGCGCTTTGGTGGTTTTA
GTATGGCCTCCTTCTGCTCGGAAATTCGCTTAGGGGTGACGCAACCCGCCAACTCCTGA

### REFERENCES

1. Alvey, R. M., Biswas, A., Schluchter, W. M., and Bryant, D. A. (2011) J. Bacteriol. 193, 1663-1671.
2. Alvey, R. M., Biswas, A., Schluchter, W. M., and Bryant, D. A. (2011) Biochemistry 50, 4890-4902.
3. Anderson, L. K., and Toole, C. M. (1998) Mol. Microbiol. 30, 467-474. Andrade, M. A., Petosa, C., O'Donoghue, S. I., Müller, C. W., and Bork, P. (2001) J. Mol. Biol. 309, 1-18.
4. Apt, K. E., J. L. Collier, and A. R. Grossman. 1995. Evolution of the Phycobiliproteins. Journal of Molecular Biology 248:79-96.
5. Arciero, D. M., Bryant, D. A., and Glazer, A. N. (1988) J. Biol. Chem. 263, 18343-18349.
6. Baier, K., Lehmann, H., Stephan, D. P., and Lockau, W. (2004) Microbiol. 150, 2739-2749.
7. Beale, S. I., and J. Cornejo. 1991. Biosynthesis of Phycobilins—3(Z)-Phycoerythrobilin and 3(Z)-Phycocyanobilin Are Intermediates in the Formation of 3(E)-Phycocyanobilin from Biliverdin-Ix-Alpha. Journal of Biological Chemistry 266:22333-22340.
8. Beale, S. I., and J. Cornejo. 1991. Biosynthesis of Phycobilins—15,16-Dihydrobiliverdin-Ix-Alpha Is a Partially Reduced Intermediate in the Formation of Phycobilins from Biliverdin-Ix-Alpha. Journal of Biological Chemistry 266:22341-22345.
9. Beale, S. I., and J. Cornejo. 1991. Biosynthesis of Phycobilins—Ferredoxin-Mediated Reduction of Biliverdin Catalyzed by Extracts of Cyanidium-Caldarium. Journal of Biological Chemistry 266:22328-22332.
10. Becker, A., M. Schmidt, W. Jager, and A. Puhler. 1995. New Gentamicin-Resistance and Lacz Promoter-Probe Cassettes Suitable for Insertion Mutagenesis and Generation of Transcriptional Fusions. Gene 162:37-39.
11. Berkelman, T. R., and Lagarias, J. C. (1986) Visualization of Bilin-Linked Peptides and Proteins in Polyacrylamide Gels, Analytical Biochemistry 156, 194-201.
12. Bhalerao, R. P., Kind, L. K., and Gustafsson, P. (1994) Plant Mol. Biol. 26, 313-326
13. Bhoo, S. H., Davis, S. J., Walker, J., Karniol, B., and Vierstra, R. D. (2001) Bacteriophytochromes are photochromic histidine kinases using a biliverdin chromophore, Nature 414, 776-779.
14. Bienert, R., Baier, K., Volkmer, R., Lockau, W., and Heinemann, U. (2006) J. Biol. Chem. 281, 5216-5223.
15. Bishop, J. E., Lagarias, J. C., Nagy, J. O., Schoenleber, R. W., Rapoport, H., Klotz, A. V., and Glazer, A. N. (1986) J. Biol. Chem. 261, 6790-6796.
16. Bishop, J. E., Rapoport, H., Klotz, A. V., Chan, C. F., Glazer, A. N., Guglistaller, P., and Zuber, H. (1987) J. Am. Chem. Soc. 109, 875-881.
17. Biswas, A., Vasquez, Y. M., Dragomani, T. M., Kronfel, M. L., Williams, S. R., Alvey, R. M., Bryant, D. A., and Schluchter, W. M. (2010) Biosynthesis of Cyanobacterial Phycobiliproteins in Escherichia coli: Chromophorylation Efficiency and Specificity of All Bilin Lyases from Synechococcus sp Strain PCC 7002, Applied and Environmental Microbiology 76, 2729-2739.
18. Biswas, A., Vasquez, Y. M., Dragomani, T. M., Kronfel, M. L., Williams, S. R., Alvey, R. M., Bryant, D. A., and Schluchter, W. M. (2010) Appl. Environ. Microbiol. 76, 2729-2739.
19. Blot, N., Wu, X. J., Thomas, J. C., Zhang, J., Garczarek, L., Bohm, S., Tu, J. M., Zhou, M., Ploscher, M., Eichacker, L., Fairchild, C. D., Zhao, J. D., Zhou, J. H., Colson, S. E., Bryant, D. A., and Glazer, A. N. (1992) Phycocyanin Alpha-Subunit Phycocyanobilin Lyase, Proc. Natl. Acad. Sci. U.S.A. 89, 7017-7021.
20. Blot, N., Wu, X. J., Thomas, J. C., Zhang, J., Garczarek, L., Bohm, S., Tu, J. M., Zhou, M., Ploscher, M., Eichacker, L., Partensky, F., Scheer, H., and Zhao, K. H. (2009) J. Biol. Chem. 284, 9290-9298.
21. Bohm, S., Endres, S., Scheer, H., and Zhao, K. H. (2007) J. Biol. Chem. 282, 25357-25366.
22. Boutaghou, M. N., Biswas, A., Cole, R. B., and Schluchter, W. M. (2010) Localization of the Specific Site of Enzyme-Mediated Covalent Binding of Phycoerythrobilin (PEB) to C-Phycoerythrin Alpha Subunit. in Proceedings of the 58th ASMS Conference on Mass Spectrometry and Allied Topics, Salt Lake City, Utah.
23. Boutaghou, M. N., Biswas, A., Schluchter, W. M., and Cole, R. B. (2011) Localization of the Specific Site of Enzyme-Mediated Covalent Binding of Phycoerythrobilin (PEB) to C-Phycoerythrin Beta Subunit. in Proceedings of the 59th ASMS Conference on Mass Spectrometry and Allied Topics, Denver, Co.
24. Bruce, D., S. Brimble, and D. A. Bryant. 1989. State transitions in a phycobilisome-less mutant of the cyanobacterium Synechococcus sp. PCC 7002. Biochimica et Biophysica Acta (BBA)—Bioenergetics 974:66-73.
25. Bryant, D. A. (1988). in Light-Energy Transduction in Photosynthesis: Higher Plant and Bacterial Models (Stevens, S. E. J., and Bryant, D. A. eds.), American Society of Plant Physiologists, Rockville, Md. pp 62-90.
26. Bryant, D. A. (1991) Cyanobacterial Phycobilisomes: Progress toward complete structural and functional analysis via molecular genetics. in Cell Culture and Somatic Cell Genetics of Plants (Bogarad, L., and Vasil, I. L. eds.), Academic Press, San Diego.
27. Bryant, D. A., Glazer, A. N., and Eiserling, F. A. (1976) Characterization and Structural-Properties of Major Biliproteins of Anabaena Sp, Archives of Microbiology 110, 61-75.
28. Bryant, D. A., Lorimier, R., Guglielmi, G., and Stevens, S. E. (1990) Arch. Microbiol. 153, 550-560.
29. Bryant, D. A., Stirewalt, V. L., Glauser, M., Frank, G., Sidler, W., and Zuber, H. (1991) Gene 107, 91-99.
30. Collier, J. L., and Grossman, A. R. (1994) EMBO J. 13, 1039-1047.
31. Cornejo, J., R. D. Willows, and S. I. Beale. 1998. Phytobilin biosynthesis: cloning and expression of a gene encoding soluble ferredoxin-dependent heme oxygenase from Synechocystis sp. PCC 6803. Plant Journal 15:99-107.
32. Dammeyer, T., Bagby, S. C., Sullivan, M. B., Chisholm, S. W., and Frankenberg-Dinkel, N. (2008) Efficient phage-mediated pigment biosynthesis in oceanic cyanabacteria, Curr. Biol. 18, 442-448.
33. Dammeyer, T., Bagby, S. C., Sullivan, M. B., Chisholm, S. W., and Frankenberg-Dinkel, N. (2008) Curr. Biol. 18, 442-448.
34. Dammeyer, T., E. Hofmann, and N. Frankenberg-Dinkel. 2008. Phycoerythrobilin synthase (PebS) of a marine virus—Crystal structures of the biliverdin complex and the substrate-free form. Journal of Biological Chemistry 283:27547-27554.
35. de Lorimier, R., Bryant, D. A., and Stevens, S. E. (1990) Biochim. Biophys. Acta 1019, 29-41.
36. de Lorimier, R., Bryant, D. A., Porter, R. D., Liu, W., Jay, E., and Stevens, S. E. (1984) Proc. Natl. Acad. Sci. USA 81, 7946-7950.
37. De Marsac, N. T., and G. Cohen-Bazire. 1977. Molecular Composition of Cyanobacterial Phycobilisomes. Proceedings of the National Academy of Sciences of the United States of America 74:1635-1639.
38. Debreczeny, M. P., K. Sauer, J. H. Zhou, and D. A. Bryant. 1993. Monomeric C-Phycocyanin at Room-Temperature and 77-K—Resolution of the Absorption and Fluorescence-Spectra of the Individual Chromophores and the Energy-Transfer Rate Constants. Journal of Physical Chemistry 97:9852-9862.
39. Dines, M., Sendersky, E., David, L., Schwarz, R., and Adir, N. (2008) J. Biol. Chem. 283, 30330-30340.
40. Everroad, C., Six, C., Partensky, F., Thomas, J. C., Holtzendorff, J., and Wood, A. M. (2006) Biochemical bases of type IV chromatic adaptation in marine Synechococcus spp, Journal of Bacteriology 188, 3345-3356.
41. Fairchild, C. D., and A. N. Glazer. 1994. Oligomeric Structure, Enzyme-Kinetics, and Substrate-Specificity of the Phycocyanin Alpha-Subunit Phycocyanobilin Lyase. Journal of Biological Chemistry 269:8686-8694.
42. Fairchild, C. D., and Glazer, A. N. (1994) J Biol Chem 269, 8686-8694.
43. Fairchild, C. D., and Glazer, A. N. (1994) J. Biol. Chem. 269, 28988-28996.
44. Fairchild, C. D., Zhao, J., Zhou, J., Colson, S. E., Bryant, D. A., and Glazer, A. N. (1992) Proc. Natl. Acad. Sci., USA 89, 7017-7021.
45. Federspiel, N. A., and Grossman, A. R. (1990) J. Bacteriol. 172, 4072-4081.
46. Fischer, A. J., Rockwell, N. C., Jang, A. Y., Ernst, L. A., Waggoner, A. S., Duan, Y., Lei, H. X., and Lagarias, J. C. (2005) Multiple roles of a conserved GAF domain tyrosine residue in cyanobacterial and plant phytochromes, Biochemistry 44, 15203-15215.
47. Frankenberg, N., Mukougawa, K., Kohchi, T., and Lagarias, J. C. (2001) Functional genomic analysis of the HY2 family of ferredoxin-dependent bilin reductases from oxygenic photosynthetic organisms, Plant Cell 13, 965-978.
48. Frigaard, N.-U., Y. Sakuragi, and D. A. Bryant. 2004. Gene Inactivation in the Cyanobacterium Synechococcus sp. PCC 7002 and the Green Sulfur Bacterium Chlorobium tepidum Using In Vitro-Made DNA Constructs and Natural Transformation, p. 325-340. In R. Carpentier (ed.), Photosynthesis Research Protocols, vol. 274.
49. Gindt, Y. M., Zhou, J. H., Bryant, D. A., and Sauer, K. (1992) J. Photochem. Photobiol. B 15, 75-89.
50. Gindt, Y. M., Zhou, J. H., Bryant, D. A., and Sauer, K. (1994) Biochim. Biophys. Acta-Bioenerg. 1186, 153-162.
51. Glazer, A. N. (1988) Meth. Enzymol. 167, 291-303.
52. Glazer, A. N. (1989) J. Biol. Chem 264, 1-4.
53. Glazer, A. N. (1994) J. Appl. Phycol. 6, 105-112.
54. Glazer, A. N., and C. S. Hixson. 1975. Characterization of R-Phycocyanin—Chromophore Content of R-Phycocyanin and C-Phycoerythrin. Journal of Biological Chemistry 250:5487-5495.
55. Glazer, A. N., Fang, S., and Brown, D. M. (1973) Spectroscopic Properties of C-Phycocyanin and of Its Alpha and Beta Subunits, J. Biol. Chem. 248, 5679-5685.
56. Glazer, A. N., G. S. Apell, C. S. Hixson, D. A. Bryant, S. Rimon, and D. M. Brown. 1976. Biliproteins of Cyanobacteria and Rhodophyta—Homologous Family of Photosynthetic Accessory Pigments. Proceedings of the National Academy of Sciences of the United States of America 73:428-431.
57. Gomez-Lojero, C., Perez-Gomez, B., Shen, G. Z., Schluchter, W. M., and Bryant, D. A. (2003) Biochemistry 42, 13800-13811.
58. Hess, W. R., Partensky, F., van der Staay, G. W. M., Garcia-Fernandez, J. M., Börner, T., and Vaulot, D. (1996) Proc. Natl. Acad. Sci. USA 93, 11126-11130 Hirose, Y., Narikawa, R., Katayama, M., and Ikeuchi, M. (2010) Proc. Natl. Acad. Sci. U.S. A. 107, 8854-8859
59. Hu, I. C., Lee, T. R., Lin, H. F., Chiueh, C. C., and Lyu, P. C. (2006) Biochemistry 45, 7092-7099.
60. Huang, B., Wang, G. C., Zeng, C. K., and Li, Z. G. (2002) Cancer Biother Radiopharm. 17, 35-42.
61. Isailovic, D., Li, H. W., and Yeung, E. S. (2004) J. Chromatogr. A 1051, 119-130.
62. Isailovic, D., Sultana, I., Phillips, G. J., and Yeung, E. S. (2006) Anal. Biochem. 358, 38-50.
63. Jung, L. J., Chan, C. F., and Glazer, A. N. (1995) Candidate Genes for the Phycoerythrocyanin Alpha-Subunit Lyase—Biochemical-Analysis of Pece and Pecf Interposon Mutants, J. Biol. Chem. 270, 12877-12884.
64. Jung, L. J., Chan, C. F., and Glazer, A. N. (1995) J. Biol. Chem. 270, 12877-12884.
65. Kahn, K., Mazel, D., Houmard, J., DeMarsac, N. T., and Schaefer, M. R. (1997) A role for cpeYZ in cyanobacterial phycoerythrin biosynthesis, Journal of Bacteriology 179, 998-1006.
66. Kahn, K., Mazel, D., Houmard, J., Tandeau de Marsac, N., and Schaefer, M. R. (1997) J. Bacteriol. 179, 998-1006.
67. Kami, C., Mukougawa, K., Muramoto, T., Yokota, A., Shinomura, T., Lagarias, J. C., and Kohchi, T. (2004) Complementation of phytochrome chromophore-deficient Arabidopsis by expression of phycocyanobilin:ferredoxin oxidoreductase, Proc. Natl. Acad. Sci. U.S.A. 101, 1099-1104.
68. Kehoe, D. M., and A. Gutu. 2006. Responding to color: The regulation of complementary chromatic adaptation. Annual Review of Plant Biology 57:127-150.
69. Kehoe, D. M., and Gutu, A. (2006) Ann. Rev. Plant Biol. 57, 127-150 Klotz, A. V., Glazer, A. N., Bishop, J. E., Nagy, J. O., and Rapoport, H. (1986) J. Biol. Chem. 261, 6797-6805.
70. Kohchi, T., Mukougawa, K., Frankenberg, N., Masuda, M., Yokota, A., and Lagarias, J. C. (2001) The arabidopsis HY2 gene encodes phytochromobilin synthase, a ferredoxin-dependent biliverdin reductase, Plant Cell 13, 425-436.
71. Kufer, W., and Bjorn, G. S. (1989) Photochromism of the Cyanobacterial Light Harvesting Biliprotein Phycoerythrocyanin, Physiologia Plantarum 75, 389-394.
72. Laemmli, U. K., and Favre, M. (1973) Maturation of Head of Bacteriophage-T4.1. DNA Packaging Events, Journal of Molecular Biology 80, 575-599.
73. Lakowicz, J. R. (1983) Principles of Fluorescence Spectroscopy, Plenum Press, New York.
74. Lichtenthaler, H. K. 1987. Chlorophylls and Carotenoids—Pigments of Photosynthetic Biomembranes. Methods in Enzymology 148:350-382.
75. MacColl, R. (1998) Cyanobacterial phycobilisomes, J. Struct. Biol. 124, 311-334. MacKinney, G. 1941. Absorption of light by chlorophyll solutions. Journal of Biological Chemistry 140:315-322.
76. Maxson, P., Sauer, K., Zhou, J. H., Bryant, D. A., and Glazer, A. N. (1989) Biochim. Biophys. Acta 977, 40-51.
77. Mazel, D., Guglielmi, G., Houmard, J., Sidler, W., Bryant, D. A., and Tandeau de Marsac, N. (1986) Nucl. Acids Res. 14, 8279-8290.
78. Miller, C. A., Leonard, H. S., Pinsky, I. G., Turner, B. M., Williams, S. R., Harrison, L., Jr., Fletcher, A. F., Shen, G., Bryant, D. A., and Schluchter, W. M. (2008) J. Biol. Chem. 283, 19293-19300.
79. Murphy, J. T., and Lagarias, J. C. (1997) The phytofluors: a new class of fluorescent protein probes, Curr. Biol. 7, 870-876.
80. Nguyen, D. C., Keller, R. A., Jett, J. H., and Martin, J. C. (1987) Anal. Chem. 59, 2158-2161.
81. Ong, L. J., and A. N. Glazer. 1987. R-Phycocyanin-Ii, a New Phycocyanin Occurring in Marine Synechococcus Species—Identification of the Terminal Energy Acceptor Bilin in Phycocyanins Journal of Biological Chemistry 262:6323-6327.
82. Ong, L. J., and Glazer, A. N. (1991) J Biol Chem 266, 9515-9527.
83. Ong, L., and A. Glazer. 1988. Structural studies of phycobiliproteins in unicellular marine cyanobacteria., p. 102-121. In S. S. Jr and a. B. DA (ed.), Light-Energy Transduction in Photosynthesis: Higher Plant and Bacterial Models. American Society of Plant Physiologists, Rockville, Md.
84. Parker, C. A., and Rees, W. T. (1960) Analyst 85, 587-600.
85. Partensky, F., Scheer, H., and Zhao, K. H. (2009) Phycourobilin in Trichromatic Phycocyanin from Oceanic Cyanobacteria Is Formed Post-translationally by a Phycoerythrobilin Lyase-Isomerase, J. Biol. Chem. 284, 9290-9298.
86. Rockwell, N. C., Su, Y. S., and Lagarias, J. C. (2006) Phytochrome structure and signaling mechanisms, Annual Review of Plant Biology 57, 837-858.
87. Sakamoto, T., and D. A. Bryant. 1998. Growth at low temperature causes nitrogen limitation in the cyanobacterium Synechococcus sp. PCC 7002. Archives of Microbiology 169:10-19.
88. Saunee, N. A., Williams, S. R., Bryant, D. A., and Schluchter, W. M. (2008) Biogenesis of phycobiliproteins—II. CpcS-I and CpcU comprise the heterodimeric bilin lyase that attaches phycocyanobilin to Cys-82 of beta-phycocyanin and Cys-81 of allophycocyanin subunits in synechococcus sp PCC 7002, J. Biol. Chem. 283, 7513-7522.
89. Saunée, N. A., Williams, S. R., Bryant, D. A., and Schluchter, W. M. (2008) J. Biol. Chem. 283, 7513-7522.
90. Scheer, H., and Zhao, K. H. (2008) Biliprotein maturation: the chromophore attachment, Mol. Microbiol. 68, 263-276.
91. Scheer, H., and Zhao, K. H. (2008) Mol. Microbiol. 68, 263-276.
92. Scheer, H., Zhao, K., Tu, J., Zhang, J., Kupka, M., Zhou, M., and Bohm, S. (2007) Photosyn. Research 91, 207-207.
93. Schirmer, T., Huber, R., Schneider, M., Bode, W., Miller, M., and Hackert, M. L. (1986) J. Mol. Biol. 188, 651-676.
94. Schmidt, M., Krasselt, A., and Reuter, W. (2006) Local protein flexibility as a prerequisite for reversible chromophore isomerization in alpha-phycoerythrocyanin, BBA-Proteins Proteomics 1764, 55-62.
95. Shen, G. Z., and D. A. Bryant. 1995. Characterization of a Synechococcus Sp Strain Pcc-7002 Mutant Lacking Photosystem. 1. Protein Assembly and Energy-Distribution in the Absence of the Photosystem-I Reaction-Center Core Complex. Photosynthesis Research 44:41-53.
96. Shen, G. Z., Saunee, N. A., Gallo, E., Begovic, Z., Schluchter, W. M., and Bryant, D. A. (2004) Identification of novel phycobiliprotein lyases in cyanobacteria. in Photosynthesis 2004 Light-Harvesting Systems Workshop (Niederman, R. A., Blankenship, R. E., Frank, H., Robert, B., and van Grondelle, R. eds.), Saint Adele, Quebec, Canada.
97. Shen, G., Saunee, N. A., Williams, S. R., Gallo, E. F., Schluchter, W. M., and Bryant, D. A. (2006) Identification and characterization of a new class of bilin lyase—The cpcT gene encodes a bilin lyase responsible for attachment of phycocyanobilin to CYS-153 on the beta-subunit of phycocyanin in Synechococcus sp PCC 7002, J. Biol. Chem. 281, 17768-17778.
98. Shen, G., Schluchter, W. M., and Bryant, D. A. (2008) Biogenesis of phycobiliproteins—I. cpcS-I and cpcU mutants of the cyanobacterium Synechococcus sp PCC 7002 define a heterodimeric phyococyanobilin lyase specific for beta-phycocyanin and allophycocyanin subunits, J. Biol. Chem. 283, 7503-7512.
99. Shen, G., Schluchter, W. M., and Bryant, D. A. (2008) J. Biol. Chem. 283, 7503-7512
100. Sidler, W. A. 2004. Phycobilisome and Phycobiliprotein Structures, p. 139-216. In D. A. Bryant (ed.), The Molecular Biology of Cyanobacteria, vol. 1. Springer Netherlands.
101. Six, C., J. C. Thomas, L. Garczarek, M. Ostrowski, A. Dufresne, N. Blot, D. J. Scanlan, and F. Partensky. 2007. Diversity and evolution of phycobilisomes in marine Synechococcus spp.: a comparative genomics study. Genome Biology 8.
102. Steglich, C., Frankenberg-Dinkel, N., Penno, S., and Hess, W. R. (2005) Environ. Microbiol. 7, 1611-1618.
103. Stevens, S. E., and R. D. Porter. 1980. Transformation in Agmenellum-Quadruplicatum. Proceedings of the National Academy of Sciences of the United States of America-Biological Sciences 77:6052-6056.
104. Storf, M., Parbel, A., Meyer, M., Strohmann, B., Scheer, H., Deng, M. G., Zheng, M., Zhou, M., and Zhao, K. H. (2001) Chromophore attachment to biliproteins: Specificity of PecE/PecF, a lyase-isomerase for the photoactive 3(1)-Cys-alpha 84-phycoviolobilin chromophore of phycoerythrocyanin, Biochemistry 40, 12444-12456.
105. Storf, M., Parbel, A., Meyer, M., Strohmann, B., Scheer, H., Deng, M. G., Zheng, M., Zhou, M., and Zhao, K. H. (2001) Biochemistry 40, 12444-12456.
106. Swanson, R. V., L. J. Ong, S. M. Wilbanks, and A. N. Glazer. 1991. Phycoerythrins of Marine Unicellular Cyanobacteria. 2. Characterization of Phycobiliproteins with Unusually High Phycourobilin Content. Journal of Biological Chemistry 266:9528-9534.
107. Takano, H., and Gusella, J. (2002) BMC Neurosci. 14, 15.
108. Tandeau de Marsac, N., and Cohen-Bazire, G. (1977) Molecular Composition of Cyanobacterial Phycobilisomes, Proc. Natl. Acad. Sci. U.S.A. 74, 1635-1639.
109. Tandeau de Marsac, N., and Houmard, J. (1988) Methods Enzymol 167, 318-328.
110. Toole, C. M., Plank, T. L., Grossman, A. R., and Anderson, L. K. (1998) Mol. Microbiol. 30, 475-486.
111. Tooley, A. J., and Glazer, A. N. (2002) Biosynthesis of the cyanobacterial light-harvesting polypeptide phycoerythrocyanin holo-alpha subunit in a heterologous host, Journal of Bacteriology 184, 4666-4671.
112. Tooley, A. J., Cai, Y. P. A., and Glazer, A. N. (2001) Biosynthesis of a fluorescent cyanobacterial C-phycocyanin holo-alpha subunit in a heterologous host, Proc. Natl. Acad. Sci. U.S.A. 98, 10560-10565.
113. Tooley, A., and Glazer, A. (2002) J. Bacteriol. 184, 4666-4671.
114. Tooley, A., Cai, Y., and Glazer, A. (2001) Proc. Natl. Acad. Sci. U.S.A. 98, 10560-10565.
115. Tripp, H. J., S. R. Bench, K. A. Turk, R. A. Foster, B. A. Desany, F. Niazi, J. P. Affourtit, and J. P. Zehr. Metabolic streamlining in an open-ocean nitrogen-fixing cyanobacterium. Nature 464:90-94.
116. Tsien, R. Y. (1998) Annu. Rev. Biochem. 67, 509-544.
117. Tu, J. M., Zhou, M., Haessner, R., Ploscher, M., Eichacker, L., Scheer, H., and Zhao, K. H. (2009) Toward a Mechanism for Biliprotein Lyases: Revisiting Nucleophilic Addition to Phycocyanobilin, J. Am. Chem. Soc. 131, 5399-+.
118. Ulijasz, A. T., Cornilescu, G., von Stetten, D., Cornilescu, C., Escobar, F. V., Zhang, J. R., Stankey, R. J., Rivera, M., Hildebrandt, P., and Vierstra, R. D. (2009) Cyanochromes Are Blue/Green Light Photoreversible Photoreceptors Defined by a Stable Double Cysteine Linkage to a Phycoviolobilin-type Chromophore, J. Biol. Chem. 284, 29757-29772.
119. Ulijasz, A. T., Cornilescu, G., von Stetten, D., Cornilescu, C., Escobar, F. V., Zhang, J. R., Stankey, R. J., Rivera, M., Hildebrandt, P., and Vierstra, R. D. (2009) J. Biol. Chem. 284, 29757-29772.
120. Wiethaus, J., Busch, A. W. U., Kock, K., Leichert, L. I., Herrmann, C., and Frankenberg-Dinkel, N. (2010) J. Biol. Chem. 285, 37561-37569.
121. Wilbanks, S. M., and Glazer, A. N. (1993) J. Biol. Chem. 268, 1226-1235.
122. Wilbanks, S. M., and Glazer, A. N. (1993) Rod Structure of a Phycoerythrin-II-Containing Phycobilisome. 1. Organization and Sequence of the Gene-Cluster Encoding the Major Phycobiliprotein Rod Components in the Genome of Marine Synechococcus-Sp-Wh8020, J. Biol. Chem. 268, 1226-1235.
123. Wu, S.-H., and Lagarias, J. C. (2000) Biochemistry 39, 13487-13495
124. Xu, Y., R. M. Alvey, P. O. Byrne, J. E. Graham, G. Shen, and D. A. Bryant. In press. Expression of genes in cyanobacteria: adaptation of endogenous plasmids as platforms for high-level gene expression in Synechococcus sp. PCC 7002. Humana Press, Totowa, N.J.
125. Zhao, J. D., and J. J. Brand. 1989. Specific Bleaching of Phycobiliproteins from Cyanobacteria and Red Algae at High-Temperature Invivo. Archives of Microbiology 152:447-452.
126. Zhao, K. H., Deng, M. G., Zheng, M., Zhou, M., Parbel, A., Storf, M., Meyer, M., Strohmann, B., and Scheer, H. (2000) Novel activity of a phycobiliprotein lyase: both the attachment of phycocyanobilin and the isomerization to phycoviolobilin are catalyzed by the proteins PecE and PecF encoded by the phycoerythrocyanin operon, Febs Letters 469, 9-13.
127. Zhao, K. H., Deng, M. G., Zheng, M., Zhou, M., Parbel, A., Storf, M., Meyer, M., Strohmann, B., and Scheer, H. (2000) FEBS Lett 469, 9-13.
128. Zhao, K. H., Ping, S., Bohm, S., Bo, S., Ming, Z., Bubenzer, C., and Scheer, H. (2005) Biochim. Biophys. Acta-Bioenerg. 1706, 81-87.
129. Zhao, K. H., Su, P., Li, J., Tu, J. M., Zhou, M., Bubenzer, C., and Scheer, H. (2006) Chromophore attachment to phycobiliprotein beta-subunits—Phycocyanobilin:Cysteine-beta 84 phycobiliprotein lyase activity of CpeS-like protein from Anabaena sp PCC7120, J. Biol. Chem. 281, 8573-8581.
130. Zhao, K. H., Su, P., Tu, J. M., Wang, X., Liu, H., Ploscher, M., Eichacker, L., Yang, B., Zhou, M., and Scheer, H. (2007) Phycobilin: cystein-84 biliprotein lyase, a near-universal lyase for cysteine-84-binding sites in cyanobacterial phycobiliproteins, Proc. Natl. Acad. Sci. U.S.A. 104, 14300-14305.
131. Zhao, K. H., Zhang, J., Tu, J. M., Boehm, S., Ploescher, M., Eichacker, L., Bubenzer, C., Scheer, H., Wang, X., and Zhou, M. (2007) J. Biol. Chem. 282, 34093-34103.
132. Zhao, K. H., Zhu, J. P., Deng, M. G., Zhou, M., Storf, M., Parbel, A., and Scheer, H. (2003) Photochromic chromopeptides derived from phycoerythrocyanin: biophysical and biochemical characterization, Photochemical & Photobiological Sciences 2, 741-748.
133. Zhou, J. H., Gasparich, G. E., Stirewalt, V. L., Delorimier, R., and Bryant, D. A. (1992) The Cpce and Cpcf Genes of Synechococcus Sp Pcc-7002—Construction and Phenotypic Characterization of Interposon Mutants, J. Biol. Chem. 267, 16138-16145.
134. Zhou, J., Gasparich, G. E., Stirewalt, V. L., de Lorimier, R., and Bryant, D. A. (1992) J. Biol. Chem. 267, 16138-16145.

All publications and patent applications cited in this specification and all references cited therein are herein incorporated by reference as if each individual publication or patent application or reference were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A modified phycobiliprotein selected from the group consisting of: HT-CpcA-PΦB, HT-CpcA-PEB, HT-CpcA-PCB, HT-CpcA-PtVB, HT-CpcA-PVB, and HT-CpcA-PUB, HT-ApcF-PEB, HT-ApcA-PEB, HT-ApcF-PΦB, HT-ApcB-PEB, HT-ApcB-PΦB, HT-ApcD-PEB, HT-ApcD-PΦB.

2. The modified phycobiliprotein of claim 1 wherein said phycobiliprotein is produced by the action of a lyase CpcE/CpcF or PecE/PecF.

3. The modified phycobiliprotein of claim 1 wherein said phycobiliprotein is produced by the action of PEB and CpcA with the lyase CpcE/CpcF.

4. The modified phycobiliprotein of claim 1 wherein said phycobiliprotein is produced by the action of PΦB and CpcA with the lyase CpcE/CpcF.

5. The modified phycobiliprotein of claim 1 wherein said phycobiliprotein is produced by the action of PCB and CpcA with the lyase PecE/PecF.

6. The modified phycobiliprotein of claim 1 wherein said phycobiliprotein is produced by the action of PEB and CpcA with the lyase PecE/PecF.

7. The modified phycobiliprotein of claim 1 wherein said phycobiliprotein is produced by the action of PΦB and CpcA with the lyase PecE/PecF.

8. A modified phycobiliprotein with a PCB chromophore attached to CpcA wherein said phycobiliprotein has an absorption maximum of 625, a fluorescence emission maximum of 646, and a fluorescence quantum yield of 0.39.

9. A modified phycobiliprotein with a PEB chromophore attached to CpcA wherein said phycobiliprotein has an absorption maximum of 556, a fluorescence emission maximum of 568, and a fluorescence quantum yield of 0.98.

10. A modified phycobiliprotein with a PΦB chromophore attached to CpcA wherein said phycobiliprotein has an absorption maximum of 637, a fluorescence emission maximum of 656, and a fluorescence quantum yield of 0.18.

11. A modified phycobiliprotein with a PVB chromophore attached to CpcA wherein said phycobiliprotein has an absorption maximum of 561, a fluorescence emission maximum of 577, and a fluorescence quantum yield of 0.14.

12. A modified phycobiliprotein with a PUB chromophore attached to CpcA wherein said phycobiliprotein has an absorption maximum of 497.

13. A modified phycobiliprotein with a PtVB chromophore attached to CpcA wherein said phycobiliprotein has an absorption maximum of 575, a fluorescence emission maximum of 590, and a fluorescence quantum yield of 0.23.

14. A method of producing a fluorescent protein in anoxic cell conditions comprising: providing a cell with a modified phycobiliprotein of claim 1 and thereafter, adding a heme component to said cell.

15. The method of claim 14 wherein said heme component is biliverdin.

16. A recombinant cell which expresses a phycobiliprotein fusion protein comprising: two or more heterodimeric lyases, an apoprotein and a bilin reductase, which components react inside the cell to form the phycobiliprotein protein in the presence of biliverdin IXα.

17. The cell of claim 16, wherein the cell further comprises a linear tetrapyrrole.

18. The cell of claim 16, wherein the lyase is heterodimeric phycoerythrocyanin α subunit phycoerythrocyanobilin lyase (PecE and PecF).

19. The cell of claim 16, wherein the cell components react in anoxic conditions.

20. The cell of claim 16, wherein the phycobiliprotein fusion protein is fluorescent.

21. The cell of claim 16, wherein the cell is a mammalian cell.

22. The cell of claim 16, wherein the cell is a yeast cell.

23. The cell of claim 16, wherein the cell is a bacterial cell.

24. The cell of claim 16, wherein the cell is an E. coli cell.

25. The cell of claim 16, wherein the cell is in vitro.

26. The cell of claim 16, wherein the cell is in situ.

27. A method for making a phycobiliprotein, comprising growing the cell of claim 16 under conditions wherein the cell expresses the phycobiliprotein.

28. The method of claim 27, further comprising the step of isolating the phycobiliprotein.

29. The method of claim 28, further comprising the step of specifically detecting the phycobiliprotein.

30. The method of claim 29, further comprising the step of specifically detecting the phycobiliprotein within the cell.

31. A method for making a phycobiliprotein protein, comprising growing the cell of claim 16 under conditions wherein the cell expresses the phycobiliprotein.

32. The method of claim 31, further comprising the step of isolating the phycobiliprotein.

33. The method of claim 32, further comprising the step of specifically detecting the phycobiliprotein.

34. The method of claim 33, further comprising the step of specifically detecting the phycobiliprotein within the cell.

35. A novel bilin lyase TeCpcS having an amino acid sequence of SEQ ID NO: 24.

36. A novel bilin lyase TcCpcS encoded by SEQ ID NO:25


SEQUENCE LISTING (25 sequences)

SEQ ID NO: 1 - DNA, 41 bp, Synechocystis PCC6803
cagaattcgtctctgctgtgtcgtataaggagttcgcagag

SEQ ID NO: 2 - DNA, 34 bp, Synechocystis PCC6803
aagtcgacgatttagccgataaattgtcctgtta

SEQ ID NO: 3 - DNA, 47 bp, Arabidopsis thaliana
attgacgtcgacaaggagcttgcatgtgactgctgaaccaattcttt

SEQ ID NO: 4 - DNA, 44 bp, Arabidopsis thaliana
gtgcggccgcgacgcttttaaagttgaattaataaatcatcaat

SEQ ID NO: 5 - DNA, 49 bp, Nostoc sp. PCC7120
attagcggccgctaggagggctaacatatgaatcaagcttcattgagcg

SEQ ID NO: 6 - DNA, 36 bp, Nostoc sp. PCC7120
gagggatcctagaagactaactcaaggcgatcgcca

SEQ ID NO: 7 - DNA, 33 bp, Synechococcus PCC 7002
gcactgatgcatattctgtgtggacatcgtagc

SEQ ID NO: 8 - DNA, 30 bp, Synechococcus PCC 7002
gggcagccatgggcgaaaaagcaatcttaa

SEQ ID NO: 9 - DNA, 41 bp, Synechococcus PCC 7002
tattccccatgggtcgacgaacaaagctttaattaccgcag

SEQ ID NO: 10 - DNA, 31 bp, Synechococcus PCC 7002
acctgagcatgcttcagccgacccctaccga

SEQ ID NO: 11 - DNA, 33 bp, Synechococcus PCC 7002
ggtgctccatggggctgctaacaaagcccgaaa

SEQ ID NO: 12 - DNA, 34 bp, Synechococcus PCC 7002
ggaggcgtcgacctagagtcgagcgaattgttag

SEQ ID NO: 13 - DNA, 31 bp, Synechococcus PCC 7335
gctgtgtcatatgtatcgcccttttcaagcg

SEQ ID NO: 14 - DNA, 32 bp, Synechococcus PCC 7335
atattgaggatccctaggcagcgatgcgagcg

SEQ ID NO: 15 - DNA, 33 bp, Arabicopsis thaliana
atccgttctagaaggtcttccgatctcctgaag

SEQ ID NO: 16 - DNA, 30 bp, Arabidopsis thaliana
cctagagtcgacgtcggccgggaagccgat

SEQ ID NO: 17 - DNA, 39 bp, Arabidopsis thaliana
ggtgctccatggctgcagggctgctaacaaagcccgaaa

SEQ ID NO: 18 - DNA, 33 bp, Arabidopsis thaliana
ccctggtcatgatctctgctgtgtcgtataagg

SEQ ID NO: 19 - DNA, 48 bp, Arabidopsis thaliana
tatgctgcagttagccgataaattgtcctgttaaatcccaaggacgag

SEQ ID NO: 20 - DNA, 18 bp, Synechococcus PCC 7002
cccgacgtcgcatgcctc

SEQ ID NO: 21 - DNA, 22 bp, Synechococcus PCC 7002
gaatactcaagctatgcatggg

SEQ ID NO: 22 - DNA, 37 bp, Synechococcus PCC 7002
tccccattagcatatgtgcataggtatggacatccgc

SEQ ID NO: 23 - DNA, 31 bp, Synechococcus PCC 7002
gaaaaactcgagggagttggcgggttgcgtc

SEQ ID NO: 24 - protein, 182 aa, Thermosynechococcus elongatus
MCIGMDIRDFFAQSAGRWFSQRTSHHLAFKQTESGKSQLTIELLSVDDPAVIALCQQYDMDPAWAVCGARVSWDGTMEWDNEKHEGSTVLVPIMDQGSRMEGKLLREMGYAEKAPVAGRFSMGSDGALTLITEYETIYSEERLWFASPNLRLRTSILKRFGGFSMASFCSEIRLGVTQPANS

SEQ ID NO: 25 - DNA, 549 bp, Thermosynechococcus elongatus
gtgtgcataggtatggacatccgcgatttttttgcccaaagtgctggccgctggttttcccaacggacgagtcatcacttggcctttaagcaaaccgagtccggcaagtctcagttaaccattgaattactgtcggtggatgatcctgcagtcattgccctgtgtcagcaatatgatatggatcccgcttgggcagtgtgcggcgcgcgggtcagttgggacggcaccatggaatgggacaatgagaagcacgaaggctcaacagtactggtacccatcatggatcagggatcgcggatggaaggaaagctcctgcgggaaatgggctacgctgaaaaagcaccggttgccggtcgcttcagcatgggcagtgatggtgccctgacattgattaccgaatacgaaaccatctattccgaagagcgcctctggtttgccagccccaatttacgtctgcgcaccagtattctcaagcgctttggtggttttagtatggcctccttctgctcggaaattcgcttaggggtgacgcaacccgccaactcctga